SEQUENCE LISTING (26 sequences)

SEQ ID NO: 1 - protein, 230 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 2 - protein, 219 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRAERHVKEDDGRKSPGEVPPGTSPACILATWPPGLLVLLWQETTLPEHCFSWTLEAGTG

SEQ ID NO: 3 - DNA, 690 bp, synthetic construct
  source 1..690: mol_type = other DNA
atggagcccctgcgcctgctgatcctgctgttcgtgaccgagctgagcggcgcccacaacaccaccgtgttccagggcgtggccggccagagcctgcaggtgagctgcccctacgacagcatgaagcactggggccgccgcaaggcctggtgccgccagctgggcgagaagggcccctgccagcgcgtggtgagcacccacaacctgtggctgctgagcttcctgcgccgctggaacggcagcaccgccatcaccgacgacaccctgggcggcaccctgaccatcaccctgcgcaacctgcagccccacgacgccggcctgtaccagtgccagagcctgcacggcagcgaggccgacaccctgcgcaaggtgctggtggaggtgctggccgaccccctggaccaccgcgacgccggcgacctgtggttccccggcgagagcgagagcttcgaggacgcccacgtggagcacagcatcagccgcagcctgctggagggcgagatccccttcccccccaccagcatcctgctgctgctggcctgcatcttcctgatcaagatcctggccgccagcgccctgtgggccgccgcctggcacggccagaagcccggcacccacccccccagcgagctggactgcggccacgaccccggctaccagctgcagaccctgcccggcctgcgcgacacc

SEQ ID NO: 4 - DNA, 690 bp, synthetic construct
  source 1..690: mol_type = other DNA
atggagcccctgaggctgctcatcctgctgtttgtgacagaactgtctggagcccacaacaccacagtgttccagggagttgctggccagtctctgcaagtgtcttgcccctatgacagcatgaagcactggggaaggaggaaggcttggtgtaggcagctgggagagaaaggaccttgccagagggtggtgagcacacacaacctgtggctgctgagcttcctcagaaggtggaatggctctacagccatcacagatgacaccctgggtggcaccctcaccatcaccttaaggaacctgcagcctcatgatgctggcctgtaccaatgccagagcctgcatggctctgaggctgataccctcaggaaggtgttggtggaggtgctggctgatcctctggatcacagggatgctggagacctgtggttcccaggagagtctgagagctttgaggatgcccatgtggagcacagcatcagcaggtctcttctggagggagagatccccttccctcccacaagcatcctgttgctgcttgcctgcatcttcctgatcaagatccttgctgcttctgctctttgggctgctgcctggcatggccagaaacctggaacacatcctccctctgaactggactgtggccatgaccctggctaccagttgcaaaccttgcctggcttgagggacacc

SEQ ID NO: 5 - DNA, 690 bp, synthetic construct
  source 1..690: mol_type = other DNA
atggagcccctgaggctgctgatcctgctgtttgtgacagaactgtctggagcccacaacaccacagtgttccagggagttgctggccagtctctgcaagtgtcttgcccctatgacagcatgaagcactggggaaggaggaaggcttggtgtaggcagctgggagagaaaggaccttgccagagggtggtgagcacacacaacctgtggctgctgagcttcctcagaaggtggaatggctctacagccatcacagatgacaccctgggtggcaccctcaccatcaccctgaggaacctgcagcctcatgatgctggcctgtaccaatgccagagcctgcatggctctgaggctgataccctcaggaaggtgttggtggaggtgctggctgatcctctggatcacagggatgctggagacctgtggttcccaggagagtctgagagctttgaggatgcccatgtggagcacagcatcagcaggtctcttctggagggagagatccccttccctcccacaagcatcctgttgctgcttgcctgcatcttcctcatcaagatccttgctgcttctgctctttgggctgctgcctggcatggccagaaacctggaacacatcctccctctgaactggactgtggccatgaccctggctaccagttgcaaaccttgcctggcttgagggacacc

SEQ ID NO: 6 - DNA, 690 bp, synthetic construct
  source 1..690: mol_type = other DNA
atggagcccctgagactgctcatcctgctgtttgtgacagaactgtctggggcccacaacaccacagtgttccagggggtggctggccagtccctccaggtgtcctgcccctatgactccatgaagcactgggggagaagaaaggcttggtgtagacagctgggggagaaagggccttgccagagagtggtgtccacacacaacctgtggctgctgtccttcctgagaagatggaatggctccacagccatcacagatgacaccctggggggcaccctcaccatcaccctgagaaacctgcagcctcatgatgctggcctgtaccagtgccagtccctgcatggctctgaggctgataccctgagaaaggtgctggtggaggtgctggctgatcctctggatcacagagatgctggggacctgtggttcccaggggagtctgagtcctttgaggatgcccatgtggagcactccatctccagatccctgctggagggggagatccccttccctcccacatccatcctgctgctgctggcctgcatcttcctcatcaagatcctggctgcttctgctctgtgggctgctgcctggcatggccagaaacctgggacacatcctccctctgaactggactgtggccatgaccctggctaccagctgcagaccctgcctggcctgagagacacc

SEQ ID NO: 7 - DNA, 283 bp, synthetic construct
  source 1..283: mol_type = other DNA
catggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 8 - DNA, 715 bp, synthetic construct
  source 1..715: mol_type = other DNA
gatatcaaactgcctgtttgggcttctcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagcttacttgccacaactgccaagcaagtttcgctgagtttgacacatggatccctgtggatcaactgccctaggactccgtttgcacccatgtgacactgttgactttgccctgacgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagccatatcgaattctgctggggctactggcag

SEQ ID NO: 9 - DNA, 2068 bp, synthetic construct
  source 1..2068: mol_type = other DNA
gaattctttgtttaggtctgtatgccatatttaatagggttatttggttctctggagtctaacttcctgagttctttgtatattttggatatcagtcctctatcagatgttgggttggtaaagatttttttcccccaatctgtgagttgctgttttgtcatattgacagtctcctttgctttacagaagctttttaatttcatgaggtcccatttgtcaattgttgatcttagagcctgagccattggtgttctgtacaggacaaattttcccctgtgccaatatgttcgaggttcttccccattttcttttctattagactcagtgtatctggttttatgtggaggtccttgatccactcggacttgagctttgtacaaggagataagaatggatcagtttgtatttttctacatcctaactgccagttgaactagcaacatttgttgaaaatgtttttttttcttccctcactggatgactttgacttctttgtcaaagatcaagtgaccataggtgtgtaggttcacttttgggtcttcaattttattccattgatctacctttctgtcattgtatcaataccatgaaggttttttaaaaaaaaatttttttaatcatattgctctgcagtacagcttgaggtcagggatggtgattcccccagaggctctttgattgttgagaacggtttttgctatcctgggttttttgttattccagatgaatttgagttgggattttgatagggaatacactgaatttgtagattgcttttggcaagatggccatctttccatcttctgaggtctttgatttctttcttcagagacttgaagttcttgtcatacagatctttcaattgcttggttagagtcacaccaagatattttatattatttgtgactattgtgaagggtgtcatttctctaatttatttctcagcccgtttatcctttgagtagaggaaaactactgatttgtttgaattaattttatatctagccactaagctgaagttgtttatcagctgtaggagttctctggtggattattttttagggttatttatgtattctaccacatcatctgcaaatagtgatatcttgacttcctcctttctaatttgtatccctttgacctccttttgttgcctaattgttctgggtagaactttgagtactatattaaatgataggggaaaagtggacagccttgggtgtggtgtgtgtggtatggtgtggtgtgtgtatggtgatgatgcatgtggtgtgtatggtgtggtgtatgtggtgtggtgtgtggtggtggtgataatgatgatggtggtctctctgtctctgtctgtctctttctatctctgcctctctctctctgtctctctctgcccccctctgtctccctctgtctgtctgtctgtctctttggtgtgatgtatgtggtgtgtgtgtattctacaaggttgacatgatgacagaatttaattttcttagcagcaagctcatggatcctggtgataaatgcagcatgactttactgaaaaggctttgtgatcttgaagagtggattgacttcactgtcggcagcacatgcaatctcacttgtttggtgtaatgaaagaagagaatgagaggtggaagggggatggtaatgttgaaaaaaagaatggtacagaggaaactgaggttggagagagatggggtagatggtaagagatggagaaagagggaaggaaatggagagaaagacagagagacagagagagacacacagagagacacacagagacagagaggaagggaaagggaaagagaaaggaagaggaagagggggaggggaaggggaaggggaaggggaagggagagggagaaatgtggacactagccagatttaagggagaaattagggggttgccagtctgtccacctctgatggtggcaactcagcagaaagctgctgggctcagtctggctttgttgagcaaccctgactccaccccttttcttccccacaaagcaagcttttaaagggaaggctttcttcattgaatgactgccacagtacgatg

SEQ ID NO: 10 - DNA, 352 bp, synthetic construct
  source 1..352: mol_type = other DNA
aatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 11 - DNA, 594 bp, synthetic construct
  source 1..594: mol_type = other DNA
ttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccc

SEQ ID NO: 12 - DNA, 235 bp, synthetic construct
  source 1..235: mol_type = other DNA
cgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 13 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 14 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 15 - DNA, 60 bp, synthetic construct
  source 1..60: mol_type = other DNA
agctctgggtatttaagcccgagtgagcacgcagggtctccattttgaagcgggaggtta

SEQ ID NO: 16 - DNA, 1229 bp, synthetic construct
  source 1..1229: mol_type = other DNA
gcagtcctcgcatgcctgagtgccctgttctgcactcctctcctccccaccctgcccatattcttcacaccctccttccctccccagtatcaaaacctaccctcagactctttctgcatgtggacatgtaaggggctggtggactctggttgcattggaagggaaggagtgctaacagtggcatcccaggcactagctggcagttgggggaagctttggggggcactggcactggtaatagcctctgaaattataagccactaattatgagcccctacagttataaaggaggaaagaacctgaggatgttcatctgcatctttggggcactctctcccctgctctgaaggtccccttgtcctcagctcttgatgggaggtgaggagcgtagagatttccgttgctgagttgaggggaattgacacaccaaatttttgtacacaattgcttcttcccgctgaggaaagcgcaccttttgctcccccagagcactggacaaacctgggcaagaggagagacggtgccaaatggagtcttgttcctgcagcttcttagatggtggtcagggggaaggcggggttctgaggcatggatgggaggtggtcagatgggagagggccacggcccatctggtctctctagttccaaaaggcaagccaccaacttcccaatcctatctttcaagcctctgtcacagtaatggttgaagatggcaggcaagggaggaccaagagagaagtcaaagcagggggctctgggggctgccccagcaacaggctggtgctctaagcccatctcccccgccctaaggagggttcccaaaatagcagcctcatgtctcccccaaaatatctccgagacgggtccttcctgaaaggggaacaaagccacagaaatagggaagctggaagttaaaggtcaggaaagtcggtaccaatgtgggcggctgcagagcaagcaagagtggcggggcagggagagccagccccaggccgagaggagaggtcccagtcccaaatgatggcaaagagatgtgcagaacagaactggagggggttttaagaccaagtgcctccagaatagacccagagaaggtcagttacttctccaaggaggcccagcaagagcaacagtgagaaaaccaaacccaggtcccaggtttcctggttcccaatttcccacaaacacatgctgtgccatccgctcccaacttgtataagaacctaagg

SEQ ID NO: 17 - DNA, 1229 bp, synthetic construct
  source 1..1229: mol_type = other DNA
gcagtcctcacttacctgagtgccctgttctgcactcctctcctccccaccctgcccatattcttcacaccctccttccctccccagtatcaaaacctaccctcagactctttctgctgtgagactgataaggggctggtggactctggttgcattggaagggaaggagtgcttacagtggcatcccaggcactagctggcagttgggggaagctttggggggcactggcactggtaatagcctctgaaattataagcctctcatttgaagcccctacagttataaaggaggaaagaacctgaggtgattcatctgcatctttggggcactctctcccctgctctgaaggtccccttgtcctcagctcttgtgaggaggtcaggagcatagagatttccattgctgagttgaggggaacttacacaccaaatttttgtacacaattgcttcttcccactgaggaaagcacaccttttgctcccccagagcactggacaaacctgggcaagaggagagacagtgcctgaaagagtcttgttcctgcagcttcttagtgagtggtcagggggaaggcagggttctgaggctgagtgaggaggtggtcagtgaggagagggccacagcccatctggtctctctagttccaaaaggcaagccaccaacttcccaatcctatctttcaagcctctgtcacagttgaagttgaagtgagcaggcaagggaggaccaagagagaagtcaaagcagggggctctgggggctgccccagcaacaggctggtgctctaagcccatctcccccaccctaaggagggttcccaaaatagcagcctctcatctcccccaaaatatctccaagacaggtccttcctgaaaggggaacaaagccacagaaatagggaagctggaagttaaaggtcaggaaagtcagtacctgtgaaggcagctgcagagcaagcaagagtggcagggcagggagagccagccccaggccaagaggagaggtcccagtccctgtgaaaagcaaagagtgtgacagaacagaactggagggggttttaagaccaagtgcctccagaatagacccagagaaggtcagttacttctccaaggaggcccagcaagagcaacagtgagaaaaccaaacccaggtcccaggtttcctggttcccaatttcccacaaacacttactgtgccatccactcccaacttgtataagaacctaagg

SEQ ID NO: 18 - DNA, 1254 bp, synthetic construct
  source 1..1254: mol_type = other DNA
attgactgaattcccgtgcggaccggcagtcctcacttacctgagtgccctgttctgcactcctctcctccccaccctgcccatattcttcacaccctccttccctccccagtatcaaaacctaccctcagactctttctgctgtgagactgataaggggctggtggactctggttgcattggaagggaaggagtgcttacagtggcatcccaggcactagctggcagttgggggaagctttggggggcactggcactggtaatagcctctgaaattataagcctctcatttgaagcccctacagttataaaggaggaaagaacctgaggtgattcatctgcatctttggggcactctctcccctgctctgaaggtccccttgtcctcagctcttgtgaggaggtcaggagcatagagatttccattgctgagttgaggggaacttacacaccaaatttttgtacacaattgcttcttcccactgaggaaagcacaccttttgctcccccagagcactggacaaacctgggcaagaggagagacagtgcctgaaagagtcttgttcctgcagcttcttagtgagtggtcagggggaaggcagggttctgaggctgagtgaggaggtggtcagtgaggagagggccacagcccatctggtctctctagttccaaaaggcaagccaccaacttcccaatcctatctttcaagcctctgtcacagttgaagttgaagtgagcaggcaagggaggaccaagagagaagtcaaagcagggggctctgggggctgccccagcaacaggctggtgctctaagcccatctcccccaccctaaggagggttcccaaaatagcagcctctcatctcccccaaaatatctccaagacaggtccttcctgaaaggggaacaaagccacagaaatagggaagctggaagttaaaggtcaggaaagtcagtacctgtgaaggcagctgcagagcaagcaagagtggcagggcagggagagccagccccaggccaagaggagaggtcccagtccctgtgaaaagcaaagagtgtgacagaacagaactggagggggttttaagaccaagtgcctccagaatagacccagagaaggtcagttacttctccaaggaggcccagcaagagcaacagtgagaaaaccaaacccaggtcccaggtttcctggttcccaatttcccacaaacacttactgtgccatccactcccaacttgtataagaacctaagg

SEQ ID NO: 19 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKVKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNFAKSANVDFTVDNNGLYTEPRPIGTRFLTRPL

SEQ ID NO: 20 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 21 - DNA, 8305 bp, synthetic construct
  source 1..8305: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttactaggttctagaaccggtgacgtctcccatggtgaagcttggatctgagatatcaaactgcctgtttgggcttctcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagcttacttgccacaactgccaagcaagtttcgctgagtttgacacatggatccctgtggatcaactgccctaggactccgtttgcacccatgtgacactgttgactttgccctgacgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagccatatcgaattctgctggggctactggcaggtaaggaggaaggaggctgaggggagggggcccctgggagggagcctgccctgggttgctaaccatctcctctctgccaaaagtccggaaagccaccatggagcccctgcgcctgctgatcctgctgttcgtgaccgagctgagcggcgcccacaacaccaccgtgttccagggcgtggccggccagagcctgcaggtgagctgcccctacgacagcatgaagcactggggccgccgcaaggcctggtgccgccagctgggcgagaagggcccctgccagcgcgtggtgagcacccacaacctgtggctgctgagcttcctgcgccgctggaacggcagcaccgccatcaccgacgacaccctgggcggcaccctgaccatcaccctgcgcaacctgcagccccacgacgccggcctgtaccagtgccagagcctgcacggcagcgaggccgacaccctgcgcaaggtgctggtggaggtgctggccgaccccctggaccaccgcgacgccggcgacctgtggttccccggcgagagcgagagcttcgaggacgcccacgtggagcacagcatcagccgcagcctgctggagggcgagatccccttcccccccaccagcatcctgctgctgctggcctgcatcttcctgatcaagatcctggccgccagcgccctgtgggccgccgcctggcacggccagaagcccggcacccacccccccagcgagctggactgcggccacgaccccggctaccagctgcagaccctgcccggcctgcgcgacacctgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattcccgtgcggaccggcagtcctcacttacctgagtgccctgttctgcactcctctcctccccaccctgcccatattcttcacaccctccttccctccccagtatcaaaacctaccctcagactctttctgctgtgagactgataaggggctggtggactctggttgcattggaagggaaggagtgcttacagtggcatcccaggcactagctggcagttgggggaagctttggggggcactggcactggtaatagcctctgaaattataagcctctcatttgaagcccctacagttataaaggaggaaagaacctgaggtgattcatctgcatctttggggcactctctcccctgctctgaaggtccccttgtcctcagctcttgtgaggaggtcaggagcatagagatttccattgctgagttgaggggaacttacacaccaaatttttgtacacaattgcttcttcccactgaggaaagcacaccttttgctcccccagagcactggacaaacctgggcaagaggagagacagtgcctgaaagagtcttgttcctgcagcttcttagtgagtggtcagggggaaggcagggttctgaggctgagtgaggaggtggtcagtgaggagagggccacagcccatctggtctctctagttccaaaaggcaagccaccaacttcccaatcctatctttcaagcctctgtcacagttgaagttgaagtgagcaggcaagggaggaccaagagagaagtcaaagcagggggctctgggggctgccccagcaacaggctggtgctctaagcccatctcccccaccctaaggagggttcccaaaatagcagcctctcatctcccccaaaatatctccaagacaggtccttcctgaaaggggaacaaagccacagaaatagggaagctggaagttaaaggtcaggaaagtcagtacctgtgaaggcagctgcagagcaagcaagagtggcagggcagggagagccagccccaggccaagaggagaggtcccagtccctgtgaaaagcaaagagtgtgacagaacagaactggagggggttttaagaccaagtgcctccagaatagacccagagaaggtcagttacttctccaaggaggcccagcaagagcaacagtgagaaaaccaaacccaggtcccaggtttcctggttcccaatttcccacaaacacttactgtgccatccactcccaacttgtataagaacctaaggcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggacctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagctt

SEQ ID NO: 22 - DNA, 8305 bp, synthetic construct
  source 1..8305: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttactaggttctagaaccggtgacgtctcccatggtgaagcttggatctgagatatcaaactgcctgtttgggcttctcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagcttacttgccacaactgccaagcaagtttcgctgagtttgacacatggatccctgtggatcaactgccctaggactccgtttgcacccatgtgacactgttgactttgccctgacgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagccatatcgaattctgctggggctactggcaggtaaggaggaaggaggctgaggggagggggcccctgggagggagcctgccctgggttgctaaccatctcctctctgccaaaagtccggaaagccaccatggagcccctgaggctgctcatcctgctgtttgtgacagaactgtctggagcccacaacaccacagtgttccagggagttgctggccagtctctgcaagtgtcttgcccctatgacagcatgaagcactggggaaggaggaaggcttggtgtaggcagctgggagagaaaggaccttgccagagggtggtgagcacacacaacctgtggctgctgagcttcctcagaaggtggaatggctctacagccatcacagatgacaccctgggtggcaccctcaccatcaccttaaggaacctgcagcctcatgatgctggcctgtaccaatgccagagcctgcatggctctgaggctgataccctcaggaaggtgttggtggaggtgctggctgatcctctggatcacagggatgctggagacctgtggttcccaggagagtctgagagctttgaggatgcccatgtggagcacagcatcagcaggtctcttctggagggagagatccccttccctcccacaagcatcctgttgctgcttgcctgcatcttcctgatcaagatccttgctgcttctgctctttgggctgctgcctggcatggccagaaacctggaacacatcctccctctgaactggactgtggccatgaccctggctaccagttgcaaaccttgcctggcttgagggacacctgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattcccgtgcggaccggcagtcctcacttacctgagtgccctgttctgcactcctctcctccccaccctgcccatattcttcacaccctccttccctccccagtatcaaaacctaccctcagactctttctgctgtgagactgataaggggctggtggactctggttgcattggaagggaaggagtgcttacagtggcatcccaggcactagctggcagttgggggaagctttggggggcactggcactggtaatagcctctgaaattataagcctctcatttgaagcccctacagttataaaggaggaaagaacctgaggtgattcatctgcatctttggggcactctctcccctgctctgaaggtccccttgtcctcagctcttgtgaggaggtcaggagcatagagatttccattgctgagttgaggggaacttacacaccaaatttttgtacacaattgcttcttcccactgaggaaagcacaccttttgctcccccagagcactggacaaacctgggcaagaggagagacagtgcctgaaagagtcttgttcctgcagcttcttagtgagtggtcagggggaaggcagggttctgaggctgagtgaggaggtggtcagtgaggagagggccacagcccatctggtctctctagttccaaaaggcaagccaccaacttcccaatcctatctttcaagcctctgtcacagttgaagttgaagtgagcaggcaagggaggaccaagagagaagtcaaagcagggggctctgggggctgccccagcaacaggctggtgctctaagcccatctcccccaccctaaggagggttcccaaaatagcagcctctcatctcccccaaaatatctccaagacaggtccttcctgaaaggggaacaaagccacagaaatagggaagctggaagttaaaggtcaggaaagtcagtacctgtgaaggcagctgcagagcaagcaagagtggcagggcagggagagccagccccaggccaagaggagaggtcccagtccctgtgaaaagcaaagagtgtgacagaacagaactggagggggttttaagaccaagtgcctccagaatagacccagagaaggtcagttacttctccaaggaggcccagcaagagcaacagtgagaaaaccaaacccaggtcccaggtttcctggttcccaatttcccacaaacacttactgtgccatccactcccaacttgtataagaacctaaggcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 23 - DNA, 8305 bp, synthetic construct
  source 1..8305: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttactaggttctagaaccggtgacgtctcccatggtgaagcttggatctgagatatcaaactgcctgtttgggcttctcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagcttacttgccacaactgccaagcaagtttcgctgagtttgacacatggatccctgtggatcaactgccctaggactccgtttgcacccatgtgacactgttgactttgccctgacgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagccatatcgaattctgctggggctactggcaggtaaggaggaaggaggctgaggggagggggcccctgggagggagcctgccctgggttgctaaccatctcctctctgccaaaagtccggaaagccaccatggagcccctgaggctgctgatcctgctgtttgtgacagaactgtctggagcccacaacaccacagtgttccagggagttgctggccagtctctgcaagtgtcttgcccctatgacagcatgaagcactggggaaggaggaaggcttggtgtaggcagctgggagagaaaggaccttgccagagggtggtgagcacacacaacctgtggctgctgagcttcctcagaaggtggaatggctctacagccatcacagatgacaccctgggtggcaccctcaccatcaccctgaggaacctgcagcctcatgatgctggcctgtaccaatgccagagcctgcatggctctgaggctgataccctcaggaaggtgttggtggaggtgctggctgatcctctggatcacagggatgctggagacctgtggttcccaggagagtctgagagctttgaggatgcccatgtggagcacagcatcagcaggtctcttctggagggagagatccccttccctcccacaagcatcctgttgctgcttgcctgcatcttcctcatcaagatccttgctgcttctgctctttgggctgctgcctggcatggccagaaacctggaacacatcctccctctgaactggactgtggccatgaccctggctaccagttgcaaaccttgcctggcttgagggacacctgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattcccgtgcggaccggcagtcctcacttacctgagtgccctgttctgcactcctctcctccccaccctgcccatattcttcacaccctccttccctccccagtatcaaaacctaccctcagactctttctgctgtgagactgataaggggctggtggactctggttgcattggaagggaaggagtgcttacagtggcatcccaggcactagctggcagttgggggaagctttggggggcactggcactggtaatagcctctgaaattataagcctctcatttgaagcccctacagttataaaggaggaaagaacctgaggtgattcatctgcatctttggggcactctctcccctgctctgaaggtccccttgtcctcagctcttgtgaggaggtcaggagcatagagatttccattgctgagttgaggggaacttacacaccaaatttttgtacacaattgcttcttcccactgaggaaagcacaccttttgctcccccagagcactggacaaacctgggcaagaggagagacagtgcctgaaagagtcttgttcctgcagcttcttagtgagtggtcagggggaaggcagggttctgaggctgagtgaggaggtggtcagtgaggagagggccacagcccatctggtctctctagttccaaaaggcaagccaccaacttcccaatcctatctttcaagcctctgtcacagttgaagttgaagtgagcaggcaagggaggaccaagagagaagtcaaagcagggggctctgggggctgccccagcaacaggctggtgctctaagcccatctcccccaccctaaggagggttcccaaaatagcagcctctcatctcccccaaaatatctccaagacaggtccttcctgaaaggggaacaaagccacagaaatagggaagctggaagttaaaggtcaggaaagtcagtacctgtgaaggcagctgcagagcaagcaagagtggcagggcagggagagccagccccaggccaagaggagaggtcccagtccctgtgaaaagcaaagagtgtgacagaacagaactggagggggttttaagaccaagtgcctccagaatagacccagagaaggtcagttacttctccaaggaggcccagcaagagcaacagtgagaaaaccaaacccaggtcccaggtttcctggttcccaatttcccacaaacacttactgtgccatccactcccaacttgtataagaacctaaggcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 24 - DNA, 8305 bp, synthetic construct
  source 1..8305: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttactaggttctagaaccggtgacgtctcccatggtgaagcttggatctgagatatcaaactgcctgtttgggcttctcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagcttacttgccacaactgccaagcaagtttcgctgagtttgacacatggatccctgtggatcaactgccctaggactccgtttgcacccatgtgacactgttgactttgccctgacgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagccatatcgaattctgctggggctactggcaggtaaggaggaaggaggctgaggggagggggcccctgggagggagcctgccctgggttgctaaccatctcctctctgccaaaagtccggaaagccaccatggagcccctgagactgctcatcctgctgtttgtgacagaactgtctggggcccacaacaccacagtgttccagggggtggctggccagtccctccaggtgtcctgcccctatgactccatgaagcactgggggagaagaaaggcttggtgtagacagctgggggagaaagggccttgccagagagtggtgtccacacacaacctgtggctgctgtccttcctgagaagatggaatggctccacagccatcacagatgacaccctggggggcaccctcaccatcaccctgagaaacctgcagcctcatgatgctggcctgtaccagtgccagtccctgcatggctctgaggctgataccctgagaaaggtgctggtggaggtgctggctgatcctctggatcacagagatgctggggacctgtggttcccaggggagtctgagtcctttgaggatgcccatgtggagcactccatctccagatccctgctggagggggagatccccttccctcccacatccatcctgctgctgctggcctgcatcttcctcatcaagatcctggctgcttctgctctgtgggctgctgcctggcatggccagaaacctgggacacatcctccctctgaactggactgtggccatgaccctggctaccagctgcagaccctgcctggcctgagagacacctgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattcccgtgcggaccggcagtcctcacttacctgagtgccctgttctgcactcctctcctccccaccctgcccatattcttcacaccctccttccctccccagtatcaaaacctaccctcagactctttctgctgtgagactgataaggggctggtggactctggttgcattggaagggaaggagtgcttacagtggcatcccaggcactagctggcagttgggggaagctttggggggcactggcactggtaatagcctctgaaattataagcctctcatttgaagcccctacagttataaaggaggaaagaacctgaggtgattcatctgcatctttggggcactctctcccctgctctgaaggtccccttgtcctcagctcttgtgaggaggtcaggagcatagagatttccattgctgagttgaggggaacttacacaccaaatttttgtacacaattgcttcttcccactgaggaaagcacaccttttgctcccccagagcactggacaaacctgggcaagaggagagacagtgcctgaaagagtcttgttcctgcagcttcttagtgagtggtcagggggaaggcagggttctgaggctgagtgaggaggtggtcagtgaggagagggccacagcccatctggtctctctagttccaaaaggcaagccaccaacttcccaatcctatctttcaagcctctgtcacagttgaagttgaagtgagcaggcaagggaggaccaagagagaagtcaaagcagggggctctgggggctgccccagcaacaggctggtgctctaagcccatctcccccaccctaaggagggttcccaaaatagcagcctctcatctcccccaaaatatctccaagacaggtccttcctgaaaggggaacaaagccacagaaatagggaagctggaagttaaaggtcaggaaagtcagtacctgtgaaggcagctgcagagcaagcaagagtggcagggcagggagagccagccccaggccaagaggagaggtcccagtccctgtgaaaagcaaagagtgtgacagaacagaactggagggggttttaagaccaagtgcctccagaatagacccagagaaggtcagttacttctccaaggaggcccagcaagagcaacagtgagaaaaccaaacccaggtcccaggtttcctggttcccaatttcccacaaacacttactgtgccatccactcccaacttgtataagaacctaaggcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggacctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagctt

SEQ ID NO: 25 - DNA, 2804 bp, synthetic construct
  source 1..2804: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtactaggttctagaaccggtgacgtctcccatggtgaagcttggatctgagatatcaaactgcctgtttgggcttctcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagcttacttgccacaactgccaagcaagtttcgctgagtttgacacatggatccctgtggatcaactgccctaggactccgtttgcacccatgtgacactgttgactttgccctgacgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagccatatcgaattctgctggggctactggcaggtaaggaggaaggaggctgaggggagggggcccctgggagggagcctgccctgggttgctaaccatctcctctctgccaaaagtccggaaagccaccatggagcccctgcgcctgctgatcctgctgttcgtgaccgagctgagcggcgcccacaacaccaccgtgttccagggcgtggccggccagagcctgcaggtgagctgcccctacgacagcatgaagcactggggccgccgcaaggcctggtgccgccagctgggcgagaagggcccctgccagcgcgtggtgagcacccacaacctgtggctgctgagcttcctgcgccgctggaacggcagcaccgccatcaccgacgacaccctgggcggcaccctgaccatcaccctgcgcaacctgcagccccacgacgccggcctgtaccagtgccagagcctgcacggcagcgaggccgacaccctgcgcaaggtgctggtggaggtgctggccgaccccctggaccaccgcgacgccggcgacctgtggttccccggcgagagcgagagcttcgaggacgcccacgtggagcacagcatcagccgcagcctgctggagggcgagatccccttcccccccaccagcatcctgctgctgctggcctgcatcttcctgatcaagatcctggccgccagcgccctgtgggccgccgcctggcacggccagaagcccggcacccacccccccagcgagctggactgcggccacgaccccggctaccagctgcagaccctgcccggcctgcgcgacacctgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 26 - DNA, 4048 bp, synthetic construct
  source 1..4048: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtactaggttctagaaccggtgacgtctcccatggtgaagcttggatctgagatatcaaactgcctgtttgggcttctcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagcttacttgccacaactgccaagcaagtttcgctgagtttgacacatggatccctgtggatcaactgccctaggactccgtttgcacccatgtgacactgttgactttgccctgacgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagccatatcgaattctgctggggctactggcaggtaaggaggaaggaggctgaggggagggggcccctgggagggagcctgccctgggttgctaaccatctcctctctgccaaaagtccggaaagccaccatggagcccctgaggctgctcatcctgctgtttgtgacagaactgtctggagcccacaacaccacagtgttccagggagttgctggccagtctctgcaagtgtcttgcccctatgacagcatgaagcactggggaaggaggaaggcttggtgtaggcagctgggagagaaaggaccttgccagagggtggtgagcacacacaacctgtggctgctgagcttcctcagaaggtggaatggctctacagccatcacagatgacaccctgggtggcaccctcaccatcaccttaaggaacctgcagcctcatgatgctggcctgtaccaatgccagagcctgcatggctctgaggctgataccctcaggaaggtgttggtggaggtgctggctgatcctctggatcacagggatgctggagacctgtggttcccaggagagtctgagagctttgaggatgcccatgtggagcacagcatcagcaggtctcttctggagggagagatccccttccctcccacaagcatcctgttgctgcttgcctgcatcttcctgatcaagatccttgctgcttctgctctttgggctgctgcctggcatggccagaaacctggaacacatcctccctctgaactggactgtggccatgaccctggctaccagttgcaaaccttgcctggcttgagggacacctgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattcccgtgcggaccggcagtcctcacttacctgagtgccctgttctgcactcctctcctccccaccctgcccatattcttcacaccctccttccctccccagtatcaaaacctaccctcagactctttctgctgtgagactgataaggggctggtggactctggttgcattggaagggaaggagtgcttacagtggcatcccaggcactagctggcagttgggggaagctttggggggcactggcactggtaatagcctctgaaattataagcctctcatttgaagcccctacagttataaaggaggaaagaacctgaggtgattcatctgcatctttggggcactctctcccctgctctgaaggtccccttgtcctcagctcttgtgaggaggtcaggagcatagagatttccattgctgagttgaggggaacttacacaccaaatttttgtacacaattgcttcttcccactgaggaaagcacaccttttgctcccccagagcactggacaaacctgggcaagaggagagacagtgcctgaaagagtcttgttcctgcagcttcttagtgagtggtcagggggaaggcagggttctgaggctgagtgaggaggtggtcagtgaggagagggccacagcccatctggtctctctagttccaaaaggcaagccaccaacttcccaatcctatctttcaagcctctgtcacagttgaagttgaagtgagcaggcaagggaggaccaagagagaagtcaaagcagggggctctgggggctgccccagcaacaggctggtgctctaagcccatctcccccaccctaaggagggttcccaaaatagcagcctctcatctcccccaaaatatctccaagacaggtccttcctgaaaggggaacaaagccacagaaatagggaagctggaagttaaaggtcaggaaagtcagtacctgtgaaggcagctgcagagcaagcaagagtggcagggcagggagagccagccccaggccaagaggagaggtcccagtccctgtgaaaagcaaagagtgtgacagaacagaactggagggggttttaagaccaagtgcctccagaatagacccagagaaggtcagttacttctccaaggaggcccagcaagagcaacagtgagaaaaccaaacccaggtcccaggtttcctggttcccaatttcccacaaacacttactgtgccatccactcccaacttgtataagaacctaaggcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

(19) United States
(12) Patent Application Publication
(10) Pub. No.: US 2024/0131192 A1
(43) Pub. Date: Apr. 25, 2024

(54) GENE THERAPY FOR TREM2-ASSOCIATED DISEASES AND DISORDERS

(71) Applicant: Eli Lilly and Company, Indianapolis, IN (US)

(72) Inventors: Sitharthan Kamalakaran, Pelham, NY (US); Priyam Raut, Jersey City, NJ (US); Anindya Kumar Sen, New York, NY (US); Benjamin Michael Shykind, New York, NY (US); Jessica Ruth Willen, Queens, NY (US); Neda Masoudi, New York, NY (US)

(21) Appl. No.: 18/479,828

(22) Filed: Oct. 2, 2023

Related U.S. Application Data
(60) Provisional application No. 63/378,330, filed on Oct. 4, 2022.

(51) Int. Cl. A61K 48/00; A61P 25/28; C07K 14/705; C12N 15/86

(52) U.S. Cl. CPC A61K 48/0058; A61P 25/28; C07K 14/70503; C12N 15/86; C12N 2750/14122; C12N 2750/14143

(57) ABSTRACT

Nucleic acids are described that encode for triggering receptor expressed on myeloid cells 2 (TREM2) and that can be used in expression constructs, vectors and gene therapies. Also described are methods of using the same for the treatment of TREM2-associated diseases and disorders, especially neurological diseases such as Alzheimer's Disease (AD), Adult-Onset Leukoencephalopathy with Axonal Spheroids and Pigmented Glia (ALSP) or Nasu-Hakola Disease (NHD).

### REFERENCE TO SEQUENCE LISTING SUBMITTED ELECTRONICALLY

The disclosure is being filed along with a Sequence Listing in ST.26 XML format. The Sequence Listing is provided as a file titled “30436 WO” created 13 Sep. 2023 and is 74.6 kilobytes (kb) in size. The Sequence Listing information in the ST.26 XML format is incorporated herein by reference in its entirety.

### TECHNICAL FIELD

The disclosure relates generally to biology and medicine, and more particularly it relates to synthetic nucleic acids and their use for treating diseases and disorders associated with triggering receptor expressed on myeloid cells 2 (TREM2), especially as a gene therapy for treating neurological disease such as Alzheimer's Disease (AD), Adult-Onset Leukoencephalopathy with Axonal Spheroids and Pigmented Glia (ALSP) or Nasu-Hakola Disease (NHD).

### BACKGROUND

TREM2 is a cell surface transmembrane glycoprotein expressed primarily in cells of myeloid lineage, including microglia. In humans, the complete absence of TREM2 has been shown to cause NHD, a rare neurodegenerative disease with late-onset dementia, demyelination and cerebral atrophy. See, e.g., Paloneva et al. (2002) Am. J. Hum. Genet. 71:656-662 and Paloneva et al. (2003) J. Exp. Med. 198:669-675.

Mutations in TREM2 also have been associated with risk of developing AD. See, e.g., Guerreiro et al. (2013) N. Engl. J. Med. 368:117-127. AD is an irreversible, progressive brain disorder characterized by the presence of abnormal protein deposits throughout the brain, which inhibit neuronal function, disrupt connections between neurons, and ultimately result in cell death. These deposits comprise plaques of amyloid-beta and tangles formed by phosphorylated-tau proteins. Individuals with mild AD experience memory loss, leading to wandering, difficulty handling money, repeating questions, and personality and behavior changes. Moreover, individuals with moderate AD exhibit increased memory loss, leading to confusion and difficulty recognizing friends and family, inability to learn new things, hallucinations, delusions, and paranoia. Furthermore, individuals with severe AD cannot communicate and are completely dependent on others for their care. Ultimately, protein plaques and tangles spread throughout the brain, leading to significant tissue shrinkage.

Mutations in CSF1R can lead to a microgliopathy known as Colony Stimulating Factor 1 Receptor (CSF1R)-Related Adult-Onset Leukoencephalopathy (CRL), which is a subclass of ALSP. Interestingly, TREM2 activates a similar signaling pathway resulting in intracellular and functional responses that phenocopy CSF1R activation. It is believed that increased cellular TREM2 can attenuate CSF1R loss-of-function and can attenuate CRL-associated pathology. See, e.g., Tchessalova et al. (2022) Alzheimer's Dement. 18:e061595.

A number of therapeutic approaches to increase TREM2 are known, including small molecules or monoclonal antibodies to modulate the downstream signaling of TREM2. More recently, even gene therapy approaches have been described to increase TREM2 and/or its activity. See, e.g., Intl. Patent Application Publication No. WO 2019/070894.

There is a need, however, for other disease-modifying therapies to increase TREM2 and/or its activity in individuals having TREM2-associated diseases and/or disorders.

### BRIEF SUMMARY

To address this need, the disclosure describes synthetic nucleic acids for use in treating TREM2-associated diseases and disorders. In one instance, the synthetic nucleic acid includes a nucleotide sequence that encodes TREM2 (i.e., a TREM2-encoding transgene) and that has at least about 90% (e.g., at least about 90%, 91%, 92, 93%, 94%, 95%, 96%, 97%, 98%, 99% or 100%) sequence identity to any one of SEQ ID NOS:3 to 6. In some instances, TREM2-encoding transgene can be codon-optimized. Alternatively or additionally, the TREM2-encoding transgene can be CpG minimized or CpG depleted. In certain instances, the nucleotide sequence for the TREM2-encoding transgene is SEQ ID NO:3, 4, 5 or 6.

In another instance, the synthetic nucleic acid can be an expression construct including a first nucleotide sequence for at least one expression control element operably linked to a second nucleotide sequence that encodes TREM2 (i.e., a TREM2-encoding transgene), where the second nucleotide sequence has at least about 90% (e.g., at least about 90%, 91%, 92, 93%, 94%, 95%, 96%, 97%, 98%, 99% or 100%) sequence identity to any one of SEQ ID NOS:3 to 6. As above, the TREM2-encoding transgene can be codon-optimized. Alternatively or additionally, the TREM2-encoding transgene can be CpG minimized or CpG depleted. In certain instances, the nucleotide sequence for the TREM2-encoding transgene is SEQ ID NO:3, 4, 5 or 6.

In some instances, the at least one expression control element can be a promoter, an enhancer, a post-transcriptional regulatory element or a polyadenylation signal. In other instances, the promoter can be a chicken β-actin (CBA) promoter or a CD68 promoter or F4/80 promoter and can include a nucleotide sequence having at least about 90% (e.g., at least about 90%, 91%, 92, 93%, 94%, 95%, 96%, 97%, 98%, 99% or 100%) sequence identity to any one of SEQ ID NOS:7 to 9, respectively. In certain instances, the nucleotide sequence for the promoter is SEQ ID NO:7, 8 or 9. In certain other instances, the nucleotide sequence for the promoter is SEQ ID NO:8.

In some instances, the enhancer can be a cytomegalovirus enhancer (CMVe) and can include a nucleotide sequence having at least about 90% (e.g., at least about 90%, 91%, 92, 93%, 94%, 95%, 96%, 97%, 98%, 99% or 100%) sequence identity to SEQ ID NO:10. In certain instances, the nucleotide sequence for the enhancer is SEQ ID NO:10.

In some instances, the post-transcriptional regulatory element can be a woodchuck hepatitis virus post-transcriptional regulatory element (WPRE) and can include a nucleotide sequence having at least about 90% (e.g., at least about 90% 91%, 92, 93%, 94%, 95%, 96%, 97%, 98%, 99% or 100%) sequence identity to SEQ ID NO:11. In certain instances, the nucleotide sequence for the post-transcriptional regulatory element is SEQ ID NO:11.

In some instances, the polyadenylation signal can be a bovine growth hormone polyA signal tail (BGHpA) and can include a nucleotide sequence having at least about 90% (e.g., at least about 90%, 91%, 92, 93%, 94%, 95%, 96%, 97%, 98%, 99% or 100%) sequence identity to SEQ ID NO:12. In certain instances, the nucleotide sequence for the polyadenylation signal is SEQ ID NO:12.

In some instances, the expression construct can include an additional nucleotide sequence that encodes a second transgene or that encodes an inhibitory nucleic acid.

In another instance, the synthetic nucleic acid can be a vector that includes an expression construct herein. In some instances, the vector can be a plasmid or viral vector. In other instances, the viral vector can be an adeno-associated virus (AAV) vector or a Baculovirus vector. When the vector is an AAV vector, it can include at least one AAV inverted terminal repeat (ITR) sequence flanking the expression construct. In some instances, the AAV ITR can be a wild-type (WT) AAV2 ITR and can have a nucleotide sequence having at least about 90% (e.g., at least about 90%, 91%, 92, 93%, 94%, 95%, 96%, 97%, 98%, 99% or 100%) sequence identity to SEQ ID NO:13 or a reverse complementary sequence thereto. In other instances, the AAV ITR can be a modified AAV2 ITR and can have a nucleotide sequence having at least about 90% (e.g., at least about 90%, 91%, 92, 93%, 94%, 95%, 96%, 97%, 98%, 99% or 100%) sequence identity to SEQ ID NO:14 or a reverse complementary sequence thereto. In certain instances, the nucleotide sequence for the AAV ITR is SEQ ID NO:13 or 14 or a reverse complementary sequence thereto.

In some instances, the vector also can include a TRY region and can have a nucleotide sequence having at least about 90% (e.g., at least about 90%, 91%, 92, 93%, 94%, 95%, 96%, 97%, 98%, 99% or 100%) sequence identity to SEQ ID NO:15. In certain instances, the nucleotide sequence for the TRY region is SEQ ID NO:15.

In some instances, the vector also can include a nucleotide sequence that assists in packaging the vector with capsid protein (i.e., a sequence that optimizes the size of the vector for packaging within capsid protein, also called a stuffer sequence). In some instances, the stuffer sequence has at least about 90% (e.g., at least about 90%, 91%, 92, 93%, 94%, 95%, 96%, 97%, 98%, 99% or 100%) sequence identity to any one of SEQ ID NOS:16 to 18. In certain instances, the nucleotide sequence is SEQ ID NO:16, 17 or 18.

The disclosure also describes a rAAV that includes (i) a rAAV vector including a nucleotide sequence having at least about 90% (e.g., at least about 90%, 91%, 92, 93%, 94%, 95%, 96%, 97%, 98%, 99% or 100%) sequence identity to any one of SEQ ID NOS:3 to 6 (i.e., TREM2-encoding transgene) and (2) an AAV6 capsid protein.

In some instances, the rAAV includes:
(a) a rAAV vector having, in 5′ to 3′ order, a nucleotide sequence including:
   (i) a first AAV ITR or a reverse complementary sequence thereto,
   (ii) a promoter,
   (iii) a TREM2-encoding transgene,
   (iv) a post-transcriptional regulatory element,
   (v) a polyadenylation signal, and
   (vii) a second AAV ITR or a reverse complementary sequence thereto; and
(b) encapsidated in an AAV6 capsid protein.

In some instances, the AAV6 capsid protein is a modified AAV6 capsid protein (e.g., an AAV6TM capsid protein) including an amino acid sequence having at least about 95% (e.g., at least about 95%, 96%, 97%, 98%, 99% or 100%) sequence identity to SEQ ID NO:19. In certain instances, the AAV6TM capsid protein amino acid sequence is SEQ ID NO:19.

In some instances, the rAAV vector includes a nucleotide sequence having at least about 90% (e.g., at least about 90%, 91%, 92, 93%, 94%, 95%, 96%, 97%, 98%, 99% or 100%) sequence identity to any one of SEQ ID NOS:21 to 26. In certain instances, the nucleotide sequence of the rAAV vector is any one of SEQ ID NOS:21, 22, 23, 24, 25 or 26.

In some instances, the rAAV vector further includes a nucleotide sequence that assists in packaging the vector with capsid protein (i.e., a stuffer sequence) having at least about 90% (e.g., at least about 90%, 91%, 92, 93%, 94%, 95%, 96%, 97%, 98%, 99% or 100%) sequence identity to any one of SEQ ID NOS:16 to 18. In certain instances, the nucleotide sequence for the stuffer sequence is SEQ ID NO:16, 17 or 18.

In some instances, the rAAV vector further includes a second nucleic acid having a nucleotide sequence having reverse complementary of any one of SEQ ID NOS:21, 22, 23, 24, 25 or 26.

This disclosure also describes a pharmaceutical composition that includes a synthetic nucleic acid, expression construct, vector or rAAV herein and a pharmaceutically acceptable carrier.

In some instances, the pharmaceutical composition can be a formulation including a rAAV herein and one or more of:
(a) about 20 mM TRIS (pH 8.0),
(b) about 1 mM MgCl₂,
(c) about 200 mM NaCl, and
(d) about 0.005% (w/v) Poloxamer 188.

In some instances, the rAAV can be present in the formulation at a concentration from about 1×10¹³ vector genomes (vg) to about 7×10¹⁴ vg. In other instances, the rAAV can be present in the formulation at a concentration of about 3.5×10¹³ vg, about 7.0×10¹³ vg or about 1.4×10¹⁴ vg.

This disclosure also describes a method of treating diseases and disorders associated with TREM2 in an individual. The method can include at least a step of administering to the individual an effective amount of a synthetic nucleic acid, expression construct, vector or rAAV herein.

In some instances, the disease or disorder associated with TREM2 can be AD, ALSP or NHD.

In some instances, the method also can include a step of measuring TREM2, CSF1R, neurofilament light chain (NfL), chitotriosidase (Chitl) and/or granulocyte-macrophage colony stimulating factor (GM-CSF) in plasma and/or cerebrospinal fluid (CSF) and/or urine from the individual and comparing an obtained value to an earlier-obtained comparable value or to a control value to assess effectiveness of the method.

In some instances, the method also can include a step of administering to the individual an effective amount of at least one additional therapeutic agent.

The disclosure further describes a composition including the rAAV for use as a medicament for treatment of diseases and disorders associated with TREM2, such as AD, ALSP or NHD.

The disclosure further describes a composition including the rAAV for use in the treatment of diseases and disorders associated with TREM2, such as AD, ALSP or NHD.

The disclosure further describes uses of the rAAV in manufacturing a medicament for treating diseases and disorders associated with TREM2, such as AD, ALSP or NHD, where the medicament optionally may include an additional therapeutic agent.

An advantage of the synthetic nucleic acids described herein is that they can increase and/or improve microglia function (i.e., development, maintenance and/or activation) in AD, ALSP or NHD by increasing both membrane-bound and secreted TREM2 levels and/or by augmenting or replacing CSF1R signaling with TREM2 signaling to activate downstream CSF1R effects.

An advantage of the rAAV described herein is that it shows broader central nervous system (CNS) biodistribution and increased microglial transduction as compared to a rAAV having AAV9 capsid protein.

### BRIEF DESCRIPTION OF THE DRAWINGS

The advantages, effects, features, and objects other than those set forth above will become more readily apparent when consideration is given to the detailed description below. Such detailed description refers to the following drawing(s), where:

FIG. 1 is a schematic depicting a first exemplary rAAV vector for expressing TREM2.

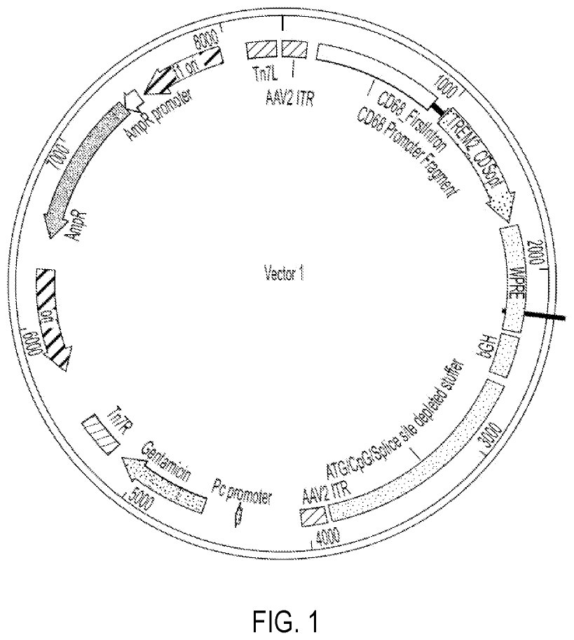

FIG. 2 is a schematic depicting a second exemplary rAAV vector for expressing TREM2.

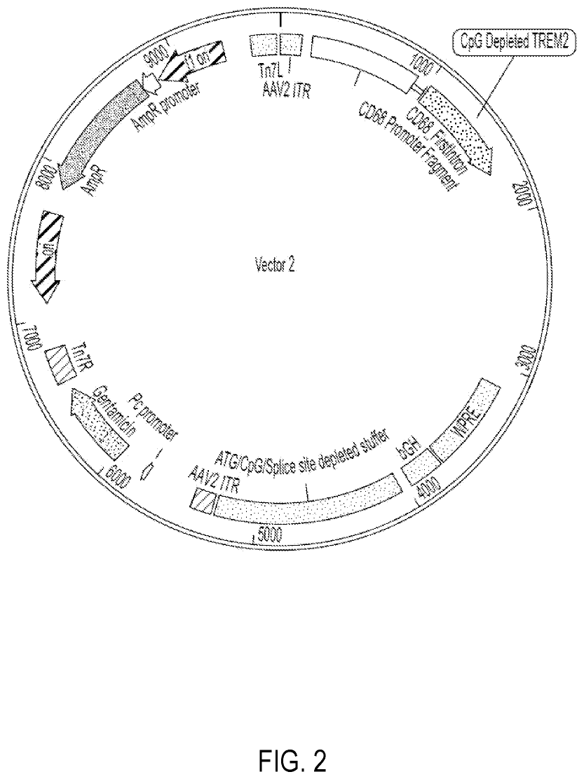

FIG. 3 is a schematic depicting a third exemplary rAAV vector for expressing TREM2.

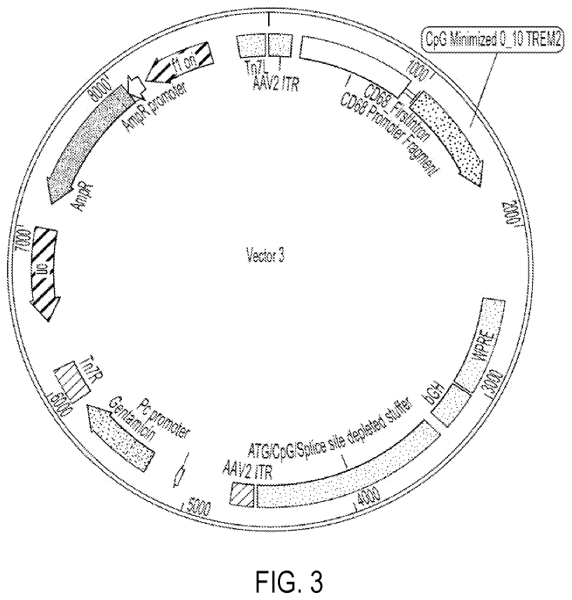

FIG. 4 is a schematic depicting a fourth exemplary rAAV vector for expressing TREM2.

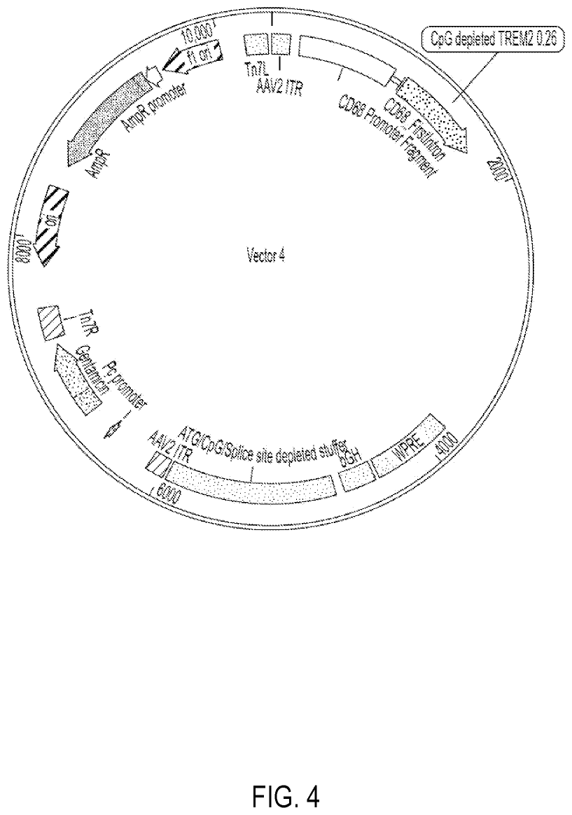

FIGS. 5A-5B show TREM2 mRNA (FIG. 5A) and protein expression (FIG. 5B) in a HMC3 cell line when transduced with an exemplary rAAV vector encapsidated in AAV6TM capsid protein as compared to the same rAAV vector encapsidated in AAV9 capsid protein (n=4/group) (AAV6TM=filled circles; AAV9=open circles).

FIGS. 6A-6B show TREM2 expression in a HMC3 cell line when transduced with one of two exemplary rAAV vectors, both encapsidated in AAV6TM capsid protein (FIG. 6A=first rAAV vector; FIG. 6B=second rAAV vector).

FIGS. 7A-D show TREM2 biodistribution in somatosensory cortex (FIG. 7A), hippocampus (FIG. 7B), cervical spinal cord (FIG. 7C) and liver (FIG. 7D) of mice administered a low or high dose of an exemplary rAAV vector encapsidated in AAV6TM capsid protein or the same rAAV vector encapsidated in AAV9 capsid protein (n=8-12/group).

FIGS. 8A-C show TREM2 protein levels in CSF (FIG. 8A), liver (FIG. 8B) and serum (FIG. 8C) of mice administered a low or high dose of an exemplary rAAV vector encapsidated in AAV6TM capsid protein or the same rAAV vector encapsidated in AAV9 capsid protein (n=8-12/group; a value of 20.48 pg/mL was set as the detection threshold (dotted line). For all graphs: statistics determined using ANOVA followed by Dunnett's test comparing to the 5×FAD+excipient group. (*) p<0.1; *p<0.05; **p<0.01; ***p<0.001; ****p<0.0001).

FIGS. 9A-9C show amyloid-β levels in the hippocampus (FIGS. 9A-9B) and X34⁺ plaques in the cortex (FIG. 9C) of mice administered a low or high dose of an exemplary rAAV vector encapsidated in AAV6TM capsid protein or the same rAAV vector encapsidated in AAV9 capsid protein (n=8-12/group; statistics determined using ANOVA followed by Dunnett's test comparing to the 5×FAD+excipient group. (*) p<0.1; *p<0.05; **p<0.01; ***p<0.001; ****p<0.0001).

FIGS. 10A-10B show inflammation (FIG. 10A for Iba and FIG. 10B for IL-10) in the cortex of mice administered a low or high dose of an exemplary rAAV vector encapsidated in AAV6TM capsid protein or the same rAAV vector encapsidated in AAV9 capsid protein (n=8-12/group; statistics determined using ANOVA followed by Dunnett's test comparing to the 5×FAD+excipient group. (*) p<0.1; *p<0.05; **p<0.01; ***p<0.001; ****p<0.0001).

FIG. 11 shows TREM2 biodistribution in non-human primates (NHPs) administered a low or high dose of an exemplary rAAV vector encapsidated in AAV6TM capsid protein (n=2/group; vector copies were measured using ddPCR).

FIG. 12A-12C show TREM2 protein levels in liver, spinal cord and dorsal root ganglion (DRG) (FIG. 12A), CSF (FIG. 12B) and serum (FIG. 12C) of NHPs administered a low or high dose of an exemplary rAAV vector encapsidated in AAV6TM capsid protein (n=2/group; a value of 20.48 pg/mL was set as the detection threshold (dotted line)).

### DETAILED DESCRIPTION

Overview

TREM2 is a cell surface transmembrane glycoprotein expressed primarily in cells of myeloid lineage, including microglia. TREM2 directly binds to amyloid-beta, facilitating microglial congregation around plaques, limiting plaque accumulation and promoting phagocytosis. TREM2 mutations associated with increased risk of AD decrease microglial response to amyloid plaques.

Additionally, CRL is a microgliopathy that can be caused by mutations in the kinase domain of CSF1R and represents the most common genetic forms of ALSP. Interestingly, CSF1R and TREM2 share a common, convergent signaling pathway to maintain and activate microglia. As such, TREM2 expression may rescue or compensate for CSF1R loss-of-function.

There are two TREM2 isoforms, where isoform 1 is 230 amino acids (aa) in length (SEQ ID NO:1; see also, NCBI Ref. Seq. No. NP 061838.1) and where isoform 2 is 219 aa in length (SEQ ID NO:2; see also, NCBI Ref. Seq. No. NP_001258750.1). Exemplary nucleic acid sequences for can be found in NCBI Ref. Seq. Nos. NM_018965.4 and NM_001271821.2 (human), NM_031254.3 and NM_001272078.1 (mouse), XP 006244486.1 and XP_006244487.1 (rat), and XP_001117305.2 and XP_001174118.2 (non-human primate). One of skill in the art, however, understands that additional examples of TREM2 mRNA sequences are readily available using publicly available databases such as, for example, GenBank and UniProt.

The disclosure describes synthetic nucleic acids for use as a gene therapy in treating TREM2-associated diseases and disorders, where the gene therapy delivers a functional copy of TREM2, which encodes TREM2, to an individual in need thereof.

### Abbreviations and Definitions

Unless defined otherwise, all technical and scientific terms used herein have the same meaning as commonly understood by one of skill in the art to which the disclosure pertains. Although any methods and materials similar to or equivalent to those described herein can be used in the practice or testing of the methods herein, the preferred methods and materials are described herein.

Additionally, reference to an element by the indefinite article “a” or “an” does not exclude the possibility that more than one element is present, unless the context clearly requires that there be one and only one element. The indefinite article “a” or “an” thus usually means “at least one.”

Moreover, use of “including,” as well as other forms, such as “including but not limited, “include,” “includes” and “included,” is not limiting.

Certain abbreviations used herein are as follows:
“aa” refers to amino acid(s); “AAV” refers to adeno-associated virus, “AcNPV” refers to Autographa californica nuclear polyhedrosis; “AD” refers to Alzheimer's Disease; “ALS” refers to amyotrophic lateral sclerosis; “ALSP” refers to adult-onset leukoencephalopathy with axonal spheroids and pigmented glia; “BAC” refers to bacterial artificial chromosome; “BEYS” refers to baculovirus vector expression system; “BBB” refers to blood-brain-barrier; “BGHpA” refers to bovine growth hormone polyA signal tail; “bp” refers to base pair(s); “CBA” refers to chicken-β actin; “Chitl” refers to chitotriosidase; “CNS” refers to central nervous system; “CSF” refers to cerebrospinal fluid; “CMVe” refers to cytomegalovirus enhancer; “CpG” refers to cytosine-phosphate-guanine; “CRL” refers to Colony Stimulating Factor 1 Receptor-Related Adult-Onset Leukoencephalopathy; “CSF1R” refers to colony stimulating factor 1 receptor gene; “CSF1R” refers to colony stimulating factor 1 receptor protein “sCSF1R” refers to soluble colony stimulating factor 1 receptor protein; “DAM” refers to disease associated microglia; “ddPCR” refers to droplet digital polymerase chain reaction; “DEA” refers to diethylamine; “DNA” refers to deoxyribonucleic acid; “DRG” refers to dorsal root ganglion; “ds” refers to double-stranded; “EDTA” refers to ethylenediaminetetraacetic acid; “EGTA” refers to ethylene glycol tetraacetic acid; “ELISA” refers to enzyme-linked immunosorbent assay; “FA” refers to formic acid; “FAD” refers to familiar Alzheimer Disease; “FTD” means frontotemporal dementia; “GC” refers to genome copy(ies); “g” refers to gram(s); “gDNA” refers to genomic DNA; “GM-CSF” refers to granulocyte-macrophage colony stimulating factor; “HMC3” refers to human microglial clone 3; “hr” refers hour(s); “ICM” refers to intra-cisterna magna; “ICV” refers to intracerebroventricular; “iPSC” refers to induced pluripotent stem cell(s); “IRES” refers to internal ribosome entry site; “ITR” refers to inverted terminal repeat; “IV” refers to intravenous; “kg” refers to kilogram(s); “min” refers to minute(s); “mL” refers to milliliter; “MSD” refers to MesoScale Discovery; “Nab” refers to neutralizing antibody(ies); “NfL” refers to neurofilament light chain; “NHD” refers to Nasu-Hakola Disease; “NHP” refers to non-human primate(s); “nt” refers to nucleotide(s); “pg” refers to picogram(s); “PLX” refers to PLX3397 (also known as pexidartinib; 5-((5-chloro-1H-pyrrolo[2,3-b]pyridin-3-yl)methyl)-N46-(trifluoromethyl)pyridin-3-yl)methyl)pyridin-2-amine or C₂₀H₁₅ClF₃N₅); “qRT-PCR” refers real-time quantitative reverse transcription polymerase chain reaction; “rAAV” refers to recombinant adeno-associated virus; “RBS” refers to Rep binding site; “RNA” refers to ribonucleic acid; “SC” refers to subcutaneous; “sec” refers to second(s); “SEM” refers to standard error of mean; “ss” refers to single-stranded; “TUB” refers to triethylammonium bicarbonate buffer; “sTREM2” refers to soluble triggering receptor expressed on myeloid cells 2 protein; “TREM2” refers to triggering receptor expressed on myeloid cells 2 gene; “TREM2” refers to triggering receptor expressed on myeloid cells 2 protein; “trs” refers to terminal resolution site; “pL” refers to microliter; “VC” refers to vector copy(ies); “vg” refers to vector genome(s); “WPRE” refers to woodchuck hepatitis virus post-transcriptional regulatory element; “WT” refers to wild-type; and “YAC” refers to yeast artificial chromosome.

Certain definitions used herein are defined as follows:

As used herein, “AAV6TM” means an AAV6TM capsid protein having at least the following 3 mutations (i.e., triple mutant) in the amino acid sequence as compared to a wild-type (WT; see, NCBI Ref. Seq. No. AAB95450.1) AAV6 capsid protein amino acid sequence: T492V, Y705F and Y731F (see, e.g., SEQ ID NO:19).

As used herein, “about” means within a statistically meaningful range of a value or values such as, for example, a stated concentration, length, molecular weight, pH, sequence similarity, time frame, temperature, volume, etc. Such a value or range can be within an order of magnitude typically within 20%, more typically within 10%, and even more typically within 5% of a given value or range. The allowable variation encompassed by “about” will depend upon the particular system under study, and can be readily appreciated by one of skill in the art.

As used herein, “administer,” “administering,” “administration” and the like mean providing a substance (e.g., a synthetic nucleic acid, expression construct, vector or rAAV herein) to an individual in a manner that is pharmacologically useful (e.g., to treat a disease, disorder, condition or symptom in the individual).

As used herein, “codon-optimized” means, with respect to a nucleotide sequence such as a gene of interest (e.g., TRM2), an alteration of codons or sequences in the gene or coding regions therein to reflect typical codon usage of a host organism (e.g., a mammal such as a human) or cell thereof without altering the polypeptide encoded by the nucleotide sequence. A codon-optimized transgene therefore is optimized for expression in a particular organism, organ, tissue or cell type, especially a mammal or mammalian organ, tissue or cell type. Alternatively, “codon-optimized” means an alteration of codons or sequences in a gene to improve protein expression as compared to a sequence that lacks the alteration by, for example, eliminating or changing sites that may be latent splice sites, stop codons, miRNA recognition sequences and the like. An entire nucleotide sequence may be codon-optimized or only one or more parts, portions or regions of a nucleotide sequence may be codon-optimized.

As used herein, “comparison window” means a contiguous and specified segment of a nucleotide sequence or amino acid sequence, where the sequence in the comparison window may include additions and/or deletions (i.e., gaps) compared to a reference sequence (which does not include the additions and/or deletions) for optimal alignment of the two sequences. Generally, the comparison window is at least 10 contiguous nucleotides/amino acids in length, and optionally can be 20, 30, 40, 50, 60, 70, 80, 90 or 100 nucleotides/amino acids, or longer.

As used herein, “complementary” means a structural relationship between two nucleotides (e.g., on two opposing nucleic acids or on opposing regions of a single nucleic acid strand) that permits the two nucleotides to form base pairs (bp) with one another. For example, a purine nucleotide of one nucleic acid that is complementary to a pyrimidine nucleotide of an opposing nucleic acid may base pair together by forming hydrogen bonds with one another. Complementary nucleotides can base pair in the Watson-Crick manner or in any other manner that allows for the formation of stable duplexes. Likewise, two nucleic acids may have regions of multiple nucleotides that are complementary with each other to form regions of complementarity, as described herein.

As used herein, “CpG depleted,” with regard to a nucleotide sequence, means that all (i.e., 100%) known CpG sites are eliminated/removed in the nucleotide sequence.

As used herein, “CpG minimized,” with regard to a nucleotide sequence, means that some (i.e., <100%) CpG sites, but not all, are eliminated/removed in the nucleotide sequence.

As used herein, “CpG site” and the like means where a cytosine (C) nucleotide is followed by a guanine (G) nucleotide in the linear sequence of bases along a 5′ to 3′ direction in a nucleotide sequence.

As used herein, “TREM2-associated disease or disorder associated” means a disease or disorder resulting from a mutation in TREM2 causing changes in TREM2 expression, TREM2 amount and/or TREM2 activity/function as compared to its expected physiology. Examples of such diseases or disorders include, but are not limited to, AD, amyotrophic lateral sclerosis (ALS), ALSP, cognitive deficit, frontotemporal dementia (FTD), NHD, memory loss, multiple sclerosis, spinal cord injury and traumatic brain injury.

As used herein, “effective amount” means an amount, concentration or dose of a therapeutic agent (e.g., a nucleic acid, expression construct, vector or rAAV herein), or a pharmaceutical composition thereof, upon single or multiple dose administration to an individual in need thereof, provides a desired effect in such an individual under diagnosis or treatment (i.e., may produce a clinically measurable difference in a condition of the individual or may prevent a worsening in the individual). An effective amount can be readily determined by one of skill in the art by using known techniques and by observing results obtained under analogous circumstances. In determining the effective amount for an individual, a number of factors are considered, including, but not limited to, the species of mammal, its size, age and general health, the specific disease, disorder, condition or symptom involved, the degree of or involvement or the severity of the disease, disorder, condition or symptom, the response of the individual, the therapeutic agent administered, the mode of administration, the bioavailability characteristics of the preparation administered, the dose regimen selected, the use of concomitant medication, and other relevant circumstances.

As used herein, “expression construct” means a nucleotide sequence capable of expressing a nucleotide sequence of interest (e.g., a transgene or an inhibitory nucleic acid) when transformed, transfected or transduced into a target cell, tissue, organ or individual. An exemplary expression construct is a vector, such as a viral vector, especially an AAV vector or a baculovirus vector. Here, an expression construct can include at least one expression control element operably linked to the nucleotide sequence of interest such as a transgene (and/or an inhibitory nucleic acid). In this manner, the expression construct can be the expression control element, such as a promoter, in operable interaction with the transgene (and/or inhibitory nucleic acid), which is capable of directing the expression of the transgene (and/or inhibitory nucleic acid) in a cell, tissue, organ or individual, especially brain tissue.

As used herein, “expression control element” means a nucleotide sequence for a promoter, polyadenylation signal, transcription or translation termination sequence, upstream regulatory domain, origin of replication, internal ribosome entry site (IRES), enhancer and the like, which collectively provide for replication, transcription and/or translation of a desired nucleic acid (e.g., a transgene or an inhibitory nucleic acid) in a cell, tissue, organ or individual. Not all of these control sequences need always be present so long as the desired nucleotide sequence is capable of being replicated, transcribed and translated in the appropriate cell, tissue, organ or individual.

As used herein, “in combination with” means administering a therapeutic agent (e.g., nucleic acid, vector, rAAV or composition herein) either simultaneously, sequentially or in a single combined formulation with one or more additional therapeutic agents.

As used herein, “individual” means any mammal, including cats, dogs, mice, rats, and primates, especially humans. Moreover, “subject” or “patient” may be used interchangeably with “individual.”

As used herein, “individual in need thereof” means a mammal, such as a human, with a condition, disease, disorder or symptom requiring treatment or therapy, including for example, those listed herein. In particular, the preferred individual to be treated is a human.

As used herein, “inhibitory nucleic acid” means a nucleic acid molecule capable of attenuating, reducing or preventing expression of a gene or mRNA. Exemplary inhibitory nucleic acids include, but are not limited to, shRNA, siRNA, miRNA, amiRNA, etc. Here, an inhibitory nucleic acid may be a nucleotide sequence encoding for an antisense sequence to a nucleotide sequence of interest.

As used herein, “nucleic acid” means a polymer of nucleotides. Although it may comprise any type of nucleotide units, the term generally applies to nucleotide polymers of deoxyribonucleic acid (DNA) or ribonucleic acid (RNA). Polynucleotide is used to include single-stranded (ss) nucleic acids, double-stranded (ds) nucleic acids, and DNA and RNA made from nucleotide or nucleoside analogues that may be identified by their sequences, which are generally presented in the 5′ to 3′ direction (as the coding strand), where the 5′ and 3′ indicate the linkages formed between the 5′ hydroxyl group of one nucleotide and the 3′-hydroxyl group of the next nucleotide. For a coding strand presented in the 5′-3′ direction, its complement (or non-coding strand) is the strand that hybridizes to that sequence according to Watson-Crick base pairing. Thus, as used herein, the complement of a nucleic acid such as a polynucleotide is the same as the “reverse complement” and describes the nucleic acid that in its natural form, would be based paired with the nucleic acid in question.

As used herein, “nucleoside” means a nucleobase-sugar combination, where the nucleobase portion is normally a heterocyclic base. The two most common classes of such heterocyclic bases are purines and pyrimidines. The sugar is normally a pentose sugar such as a ribose or a deoxyribose (e.g., 2′-deoxyribose).

As used herein, “nucleotide” means an organic molecule having a nucleoside (a nucleobase such as, for example, adenine, cytosine, guanine, thymine or uracil; and a pentose sugar such as, e.g., ribose or 2′-deoxyribose) and a phosphate group, which can serve as a monomeric unit of nucleic acid polymers such as DNA and RNA.

As used herein, “oligonucleotide” means a short nucleic acid molecule (e.g., less than about 100 nucleotides in length). An oligonucleotide may be ss or ds.

As used herein, “operably linked” and the like means that the elements of an expression construct (or other nucleic acid construct) are configured to perform their usual function (i.e., under the influence of an expression control element). Thus, an expression control element (e.g., a promoter) operably linked to a desired nucleotide sequence (e.g., a transgene or an inhibitory nucleic acid) is capable of effecting expression of the desired nucleic acid. The control element need not be contiguous with the desired nucleotide sequence, so long as it functions to direct the expression thereof (i.e., maintain proper reading frame). Thus, for example, intervening untranslated, yet transcribed, sequence can be present between a promoter and the desired nucleotide sequence, and the promoter still can be considered “operably linked” to the desired nucleotide sequence.

As used herein, “pharmaceutically acceptable,” when referring to a material such as a carrier or diluent, means that it does not abrogate the biological activity or properties of a therapeutic agent (e.g., a nucleic acid, expression construct, vector, rAAV or composition herein) and is relatively non-toxic (i.e., the material may be administered to an individual without causing undesirable biological effects or interacting in a deleterious manner with any of the components of the composition in which it is contained.

As used herein, “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a therapeutic agent within or to an individual such that it may perform its intended function. Additional ingredients that may be included in the pharmaceutical compositions used in the practice of the invention are known in the art and described, for example in Remington's Pharmaceutical Sciences, 21′ Edition, University of the Sciences in Philadelphia, PA (2006).

As used herein, “pharmaceutical composition” means a composition or therapeutic agent (e.g., a nucleic acid, expression construct, vector, rAAV or composition herein), mixed with at least one pharmaceutically acceptable chemical component, such as, but not limited to carriers, stabilizers, diluents, dispersing agents, suspending agents, thickening agents, excipients and the like.

As used herein, “recombinant adeno-associated virus,” “recombinant AAV” and “rAAV” mean viral particles comprising a rAAV vector encapsidated by AAV capsid protein.

As used herein, “recombinant adeno-associated virus vector,” “recombinant AAV vector” and “rAAV vector” mean a synthetic polynucleotide vector comprising one or more heterologous sequences (i.e., nucleic acid sequence not of an AAV origin) that are flanked by at least one AAV ITR sequence. Such rAAV vectors can be replicated and packaged into infectious viral particles when present in a host cell that has been infected with a suitable helper virus (or that is expressing suitable helper functions) that expresses AAV rep and cap gene products (i.e., AAV Rep and Cap proteins).

As used herein, “sequence identity,” in the context of two nucleotide sequences or two amino acid sequences, means that residues in the two sequences that are the same when aligned for maximum correspondence over a specified comparison window.

As used herein, “synthetic” means a nucleic acid or other molecule or compound that is artificially synthesized (e.g., using a machine such as, for example, a solid phase nucleic acid synthesizer) or that is recombinantly produced (i.e., not naturally derived from a natural source that normally produces the nucleic acid or other compound).

As used herein, “transgene” means a nucleotide sequence that is introduced into a cell and is capable of being transcribed into RNA and optionally, translated and/or expressed under appropriate conditions. The transgene confers a desired property to a cell into which it was introduced, or otherwise leads to a desired therapeutic or diagnostic outcome. Here, the transgene can be a nucleotide sequence encoding for a polypeptide of interest such as, for example, TREM2.

As used herein, “treat,” “to treat,” “treatment” or “treating” mean a process where there may be a slowing, controlling, delaying or stopping of the progression of the diseases or disorders disclosed herein, or ameliorating disease or disorder symptoms, but does not necessarily indicate a total elimination of all disease or disorder symptoms. Treatment and the like includes administration of a nucleic acid, expression construct, vector, rAAV or composition herein for treatment of a disease or disorder in an individual, particularly in a human.

As used herein, “TREM2” means human triggering receptor expressed on myeloid cells-2 (also known as PLOSL2, TREM-2, Trem2a, Trem2b or Trem2c).

As used herein, “vector” means a recombinant plasmid or recombinant virus that includes an oligonucleotide or polynucleotide to be delivered into a host cell, either in vitro or in vivo. Examples of vectors include, but are not limited to, bacterial artificial chromosome (BAC), cosmid, phagemid, plasmid, viral vector and yeast artificial chromosome (YAC).

As used herein, “viral vector” means a vector that is derived from a naturally occurring or modified virus, especially a rAAV vector or a Baculovirus vector (e.g., Autographa californica nuclear polyhedrosis (AcNPV) vector)).

Compositions

Synthetic Nucleic Acids

TREM2-Encoding Transgenes: The synthetic nucleic acid herein can be a transgene that includes a nucleotide sequence encoding TREM2 (i.e., TREM2-encoding transgene). The TREM2-encoding transgene can be codon-optimized and/or CpG minimized and/or CpG depleted. While a CpG depleted nucleotide sequence has 100% of CpG sites eliminated/removed, a CpG minimized nucleotide sequence has <100% of CpG sites eliminated/removed. For example, a CpG minimized nucleotide sequence can have from about 1% to about 99%, from about 10% to about 90%, from about 20% to about 80%, from about 30% to about 70%, from about 40% to about 60% or about 50% CpG sites removed. Alternatively, a CpG minimized nucleotide sequence can have from about 5% to about 10%, about 10% to about 20%, about 20% to about 30%, about 40% to about 50%, about 50% to about 60%, about 60% to about 70%, about 70% to about 80%, about 80% to about 90%, or about 90% to about 99% CpG eliminated/removed. Alternatively, a CpG minimized nucleotide sequence can have about 1%, about 5%, about 10% about 15%, about 20%, about 25%, about 30%, about 35%, about 40%, about 45%, about 50%, about 55%, about 60%, about 65%, about 70%, about 75%, about 80%, about 85%, about 90%, about 95% or about 99% CpG sites eliminated/removed.

In some instances, the TREM2-encoding transgene includes a nucleotide sequence having at least about 90% sequence identity to SEQ ID NO:3. Alternatively, the nucleotide sequence has at least about 90%, about 91%, about 92%, about 93%, about 94%, about 95%, about 96%, about 97%, about 98%, about 99% or about 100% sequence identity to SEQ ID NO:3. In certain instances, the nucleotide sequence for the TREM2-encoding transgene is SEQ ID NO:3, which is a codon-optimized sequence.

In other instances, the TREM2-encoding transgene includes a nucleotide sequence having at least about 90% sequence identity to SEQ ID NO:4. Alternatively, the nucleotide sequence has at least about 90%, about 91%, about 92%, about 93%, about 94%, about 95%, about 96%, about 97%, about 98%, about 99% or about 100% sequence identity to SEQ ID NO:4. In certain instances, the nucleotide sequence for the TREM2-encoding transgene is SEQ ID NO:4, which is not only a codon-optimized but also a CpG-depleted sequence.

In some instances, the TREM2-encoding transgene includes a nucleotide sequence having at least about 90% sequence identity to SEQ ID NO:5. Alternatively, the nucleotide sequence has at least about 90%, about 91%, about 92%, about 93%, about 94%, about 95%, about 96%, about 97%, about 98%, about 99% or about 100% sequence identity to SEQ ID NO:5. In certain instances, the nucleotide sequence for the TREM2-encoding transgene is SEQ ID NO:5, which is not only a codon-optimized but also a 10% CpG-minimized sequence.

In some instances, the TREM2-encoding transgene includes a nucleotide sequence having at least about 90% sequence identity to SEQ ID NO:6. Alternatively, the nucleotide sequence has at least about 90%, about 91%, about 92%, about 93%, about 94%, about 95%, about 96%, about 97%, about 98%, about 99% or about 100% sequence identity to SEQ ID NO:6. In certain instances, the nucleotide sequence for the TREM2-encoding transgene is SEQ ID NO:6, which is not only a codon-optimized but also a 25% CpG-minimized sequence.

The synthetic nucleic acids herein may exist on their own or may exist as part of an expression construct, a vector or a rAAV as further described herein.

Expression Constructs: As noted above, synthetic nucleic acids such as a TREM2-encoding transgenes can be incorporated in an expression construct. In some instances, the expression construct can include at least one expression control element operably linked to a TREM2-encoding transgene having a nucleotide sequence of any one of SEQ ID NOS:3 to 6 (or a nucleotide sequence with at least about 90% to about 99% sequence identity thereto). In certain instances, the nucleotide sequence for the TREM2-encoding transgene is SEQ ID NO:3, 4, 5 or 6.

The at least one expression control element can be at least one transcription factor binding site, at least one repressor binding site, at least one promoter, at least one enhancer, at least one intron splice site, at least one post-transcriptional regulatory element, at least one polyadenylation signal, or combinations thereof.

When the at least one expression control element is a promoter, the promoter can be a CBA promoter or a CD68 promoter or a F4/80 promoter. In some instances, the promoter is the CBA promoter and has a nucleotide sequence having at least about 90% sequence identity to SEQ ID NO:7. In other instances, the promoter is the CD68 promoter and has a nucleotide sequence having at least about 90% sequence identity to SEQ ID NO:8. In other instances, the promoter is the F4/80 promoter and has a nucleotide sequence having at least about 90% sequence identity to SEQ ID NO:9 (see also, Intl. Patent Application Publication No. WO 2006/122141). In certain instances, the nucleotide sequence for the promoter is SEQ ID NO:8.

When the at least one expression control element is an enhancer, the enhancer can be a CMVe and has a nucleotide sequence having at least about 90% sequence identity to SEQ ID NO:10. In certain instances, the nucleotide sequence for the enhancer is SEQ ID NO:10

When the at least one expression control element is a post-transcriptional regulatory element, the post-transcriptional regulatory element can be a WPRE and has a nucleotide sequence having at least about 90% sequence identity to SEQ ID NO:11. In certain instances, the nucleotide sequence for the post-transcriptional regulatory element is SEQ ID NO:11

When the at least one expression control element is a polyadenylation signal, the polyadenylation signal can be BGHpA tail and has a nucleotide sequence having at least about 90% sequence identity to SEQ ID NO:12. In certain instances, the nucleotide sequence for the polyadenylation signal is SEQ ID NO:12.

In some instances, the expression construct also can include a nucleotide sequence for one or more of an internal ribosomal entry site (IRES), a self-cleaving peptide coding sequence such as a T2A peptide.

In some instances, the expression construct includes at least one additional nucleotide sequence for another transgene and/or an inhibitory nucleic acid.

The expression constructs herein may exist on their own or may exist as part of a vector or even a rAAV as further described herein.

Vectors: As noted above, the synthetic nucleic acids or expression constructs can be incorporated into a vector, especially a viral vector such as an rAAV vector. A rAAV vector may comprise either the “plus strand” or the “minus strand” of the rAAV vector. In some instances, the rAAV vector is ss (e.g., ss DNA or ss RNA). In other instances, the rAAV vector is ds (e.g., ds DNA or ds RNA).

To aid in expression, rAAV vectors include ITRs that flank the nucleotide sequence of interest (e.g., a TREM2-encoding transgene). In some instances, the ITR sequences are full-length (i.e., are about 145 nt in length and contain a functional Rep binding site (RBS) and a terminal resolution site (trs)) and is the WT AAV2 ITR including a nucleotide sequence having at least about 90% sequence identity to SEQ ID NO:13 or a reverse complementary sequence thereto. In certain instances, the nucleotide sequence for WT AAV2 ITR is SEQ ID NO:13 or a reverse complementary sequence thereto. In other instances, the ITR is a modified ITR (i.e., includes an addition, deletion, substitution, etc.) and is a modified AAV2 ITR including a nucleotide sequence having at least about 90% sequence identity to SEQ ID NO:14 a reverse complementary sequence thereto. In certain instances, the nucleotide sequence for the modified AAV2 ITR is SEQ ID NO: 13 or 14 or a reverse complementary sequence thereto.

The rAAV vectors also can include a TRY region as described in Francois et al. (2005) J Viral. 79:11082-11094, which can be located between an ITR (e.g., a 5′ ITR) and the TREM2-encoding transgene. In some instances, the TRY region includes a nucleotide sequence having at least about 90% sequence identity to SEQ ID NO:15. In certain instances, the nucleotide sequence for the TRY region is SEQ ID NO:15.

Additional sequences can be included in the rAAV vector to assist in packaging the rAAV vector into capsid protein (i.e., a sequence that optimizes the size of the vector for packaging within capsid protein; a “stuffer sequence”). In some instances, such a stuffer sequence includes a nucleotide sequence having at least about 90% sequence identity to SEQ ID NO:16 to 18. In certain instances, the nucleotide sequence for the stuffer sequence is SEQ ID NO:16, 17 or 18.

The vectors herein may exist on their own or may exist as part of a rAAV as further described herein.

rAAV

As noted above, the vectors can be incorporated into a rAAV (i.e., rAAV vector encapsidated in AAV capsid protein). Here, the rAAV include a capsid protein that readily spreads through the CNS, particularly when introduced into the CSF space or directly into the brain parenchyma. Examples of capsid proteins that can cross the blood-brain barrier include, but are not limited to, a capsid protein having an AAV6, AAV9 or AAVrh.10 serotype.

Of particular interest herein is rAAV that can infect microglia via AAV6-based capsid proteins, especially AAV6TM capsid protein. In some instances, the AAV6TM capsid protein includes an amino acid sequence having at least about 95% sequence identity to SEQ ID NO:19. In certain instances, the amino acid sequence for the AAV6TM capsid protein is SEQ ID NO:19.

In this manner, an rAAV herein includes (a) an AAV vector including a TREM2-encoding transgene and (b) an AAV6 capsid protein.

In some instances, the rAAV includes:
(a) a rAAV vector having, in 5′ to 3′ order, a nucleotide sequence of:
   (i) a first AAV ITR or a reverse complementary sequence thereto,
   (ii) a promoter,
   (iii) a TREM2-encoding transgene,
   (iv) a post-transcriptional regulatory element,
   (v) a polyadenylation signal, and
   (vi) a second AAV ITR a reverse complementary sequence thereto; and
(b) encapsidated in AAV6 capsid protein.

In other instances, the rAAV includes:
(a) a rAAV vector having, in 5′ to 3′ order, a nucleotide sequence of:
   (i) a first AAV2 ITR having a nucleotide sequence of SEQ ID NO:13 or 14, or a reverse complementary sequence thereto,
   (ii) a promoter having a nucleotide sequence of SEQ ID NO:8,
   (iii) a TREM2-encoding transgene having a nucleotide sequence of SEQ ID NO:3,
   (iv) a post-transcriptional regulatory element having a nucleotide sequence of SEQ ID NO:11,
   (v) a polyadenylation signal having a nucleotide sequence of SEQ ID NO:12, and
   (vi) a second AAV2 ITR having a nucleotide sequence of SEQ ID NO:13 or 14, or a reverse complementary sequence thereto; and
(b) encapsidated in AAV6 capsid protein, especially AAV6TM capsid protein having an amino acid sequence of SEQ ID NO:19.

In yet other instances, the rAAV includes:
(a) a rAAV vector having, in 5′ to 3′ order, a nucleotide sequence of:
   (ii) a first AAV2 ITR having a nucleotide sequence of SEQ ID NO:13 or 14, or a reverse complementary sequence thereto,
   (ii) a promoter having a nucleotide sequence of SEQ ID NO:8,
   (iii) a TREM2-encoding transgene having a nucleotide sequence of SEQ ID NO:4,
   (iv) a post-transcriptional regulatory element having a nucleotide sequence of SEQ ID NO:11,
   (v) a polyadenylation signal having a nucleotide sequence of SEQ ID NO:12, and
   (vi) a second AAV2 ITR having a nucleotide sequence of SEQ ID NO:13 or 14, or a reverse complementary sequence thereto; and
(b) encapsidated in AAV6 capsid protein, especially AAV6TM capsid protein having an amino acid sequence of SEQ ID NO:19.

In yet other instances, the rAAV includes:
(a) a rAAV vector having, in 5′ to 3′ order, a nucleotide sequence of:
   (i) a first AAV2 ITR having a nucleotide sequence of SEQ ID NO:13 or 14, or a reverse complementary sequence thereto,
   (ii) a promoter having a nucleotide sequence of SEQ ID NO:8,
   (iii) a TREM2-encoding transgene having a nucleotide sequence of SEQ ID NO:5,
   (iv) a post-transcriptional regulatory element having a nucleotide sequence of SEQ ID NO:11,
   (v) a polyadenylation signal having a nucleotide sequence of SEQ ID NO:12, and
   (vi) a second AAV2 ITR having a nucleotide sequence of SEQ ID NO:13 or 14, or a reverse complementary sequence thereto; and
(b) encapsidated in AAV6 capsid protein, especially AAV6TM capsid protein having an amino acid sequence of SEQ ID NO:19.

In yet other instances, the rAAV includes:
(a) a rAAV vector having, in 5′ to 3′ order, a nucleotide sequence of:
   (i) a first AAV2 ITR having a nucleotide sequence of SEQ ID NO:13 or 14, or a reverse complementary sequence thereto,
   (ii) a promoter having a nucleotide sequence of SEQ ID NO:8,
   (iii) a TREM2-encoding transgene having a nucleotide sequence of SEQ ID NO:6,
   (iv) a post-transcriptional regulatory element having a nucleotide sequence of SEQ ID NO:11,
   (v) a polyadenylation signal having a nucleotide sequence of SEQ ID NO:12, and
   (vi) a second AAV2 ITR having a nucleotide sequence of SEQ ID NO:13 or 14, or a reverse complementary sequence thereto; and
(b) encapsidated in AAV6 capsid protein, especially AAV6TM capsid protein having an amino acid sequence of SEQ ID NO:19.

Any of the rAAV above further can include a stuffer sequence having a nucleotide sequence of SEQ ID NO:16 to 18. In some instances, the stuffer sequence is positioned between the polyadenylation signal and the second AAV2 ITR.

In certain instances, the rAAV includes a rAAV vector having a nucleotide sequence of SEQ ID NO:21 encapsidated in AAV6 capsid protein, especially AAV6TM capsid protein having an amino acid sequence of SEQ ID NO:19. In some instances, the rAAV includes a reverse complementary nucleotide sequence to SEQ ID NO:21.

In certain other instances, the rAAV includes a rAAV vector having a nucleotide sequence of SEQ ID NO:22 encapsidated in AAV6 capsid protein, especially AAV6TM capsid protein (e.g., VP1) having an amino acid sequence of SEQ ID NO:19. In some instances, the rAAV includes a reverse complementary nucleotide sequence to SEQ ID NO:22.

In certain other instances, the rAAV includes a rAAV vector having a nucleotide sequence of SEQ ID NO:23 encapsidated in AAV6 capsid protein, especially AAV6TM capsid protein having an amino acid sequence of SEQ ID NO:19. In some instances, the rAAV includes a reverse complementary nucleotide sequence to SEQ ID NO:23.

In certain other instances, the rAAV includes a rAAV vector having a nucleotide sequence of SEQ ID NO:24 encapsidated in AAV6 capsid protein, especially AAV6TM capsid protein having an amino acid sequence of SEQ ID NO:19. In some instances, the rAAV includes a reverse complementary nucleotide sequence to SEQ ID NO:24.

In certain other instances, the rAAV includes a rAAV vector having a nucleotide sequence of SEQ ID NO:25 encapsidated in AAV6 capsid protein, especially AAV6TM capsid protein having an amino acid sequence of SEQ ID NO:19. In some instances, the rAAV includes a reverse complementary nucleotide sequence to SEQ ID NO:25.

In certain other instances, the rAAV includes a rAAV vector having a nucleotide sequence of SEQ ID NO:26 encapsidated in AAV6 capsid protein, especially AAV6TM capsid protein having an amino acid sequence of SEQ ID NO:19. In some instances, the rAAV includes a reverse complementary nucleotide sequence to SEQ ID NO:26.

Pharmaceutical Compositions

The synthetic nucleic acids described herein (i.e., an expression construct or a vector) or rAAVs described herein can be formulated as a pharmaceutical composition including the synthetic nucleic acid or rAAV and a pharmaceutically acceptable carrier. Pharmaceutical compositions can be prepared by methods well known in the art (e.g., Remington: The Science and Practice of Pharmacy, 22^{nd} ed. (Pharmaceutical Press, 2013).

In some instances, the pharmaceutical composition can be in the form of a formulation that includes not only a rAAV herein but also one or more of (a) about 10 mM to about 30 mM TRIS buffer, (b) about 0.5 mM to about 1.5 mM MgCl₂, (c) about 100 mM to about 300 mM NaCl and (d) about 0.001% (w/v) to about 0.01% (w/v) Poloxamer 188.

In some instances, the rAAV includes a rAAV vector having a nucleotide sequence of SEQ ID NO:21 encapsidated in AAV6 capsid protein, especially AAV6TM capsid protein (e.g., VP1) having an amino acid sequence of SEQ ID NO:19. In some instances, the rAAV includes a reverse complementary nucleotide sequence to SEQ ID NO:21. In other instances, the rAAV includes a rAAV vector having a nucleotide sequence of SEQ ID NO:22 encapsidated in AAV6 capsid protein, especially AAV6TM capsid protein having an amino acid sequence of SEQ ID NO:19. In some instances, the rAAV includes a reverse complementary nucleotide sequence to SEQ ID NO:22. In other instances, the rAAV includes a rAAV vector having a nucleotide sequence of SEQ ID NO:23 encapsidated in AAV6 capsid protein, especially AAV6TM capsid protein having an amino acid sequence of SEQ ID NO:19. In some instances, the rAAV includes a reverse complementary nucleotide sequence to SEQ ID NO:23. In other instances, the rAAV includes a rAAV vector having a nucleotide sequence of SEQ ID NO:24 encapsidated in AAV6 capsid protein, especially AAV6TM capsid protein having an amino acid sequence of SEQ ID NO:19. In some instances, the rAAV includes a reverse complementary nucleotide sequence to SEQ ID NO:24. In other instances, the rAAV includes a rAAV vector having a nucleotide sequence of SEQ ID NO:25 encapsidated in AAV6 capsid protein, especially AAV6TM capsid protein having an amino acid sequence of SEQ ID NO:19. In some instances, the rAAV includes a reverse complementary nucleotide sequence to SEQ ID NO:25. In other instances, the rAAV includes a rAAV vector having a nucleotide sequence of SEQ ID NO:26 encapsidated in AAV6 capsid protein, especially AAV6TM capsid protein having an amino acid sequence of SEQ ID NO:19. In some instances, the rAAV includes a reverse complementary nucleotide sequence to SEQ ID NO:26.

In some instances, the TRIS buffer can be at a concentration from about 10 mM to about 30 mM. In other instances, the TRIS buffer can be at a concentration from about 15 mM to about 25 mM, or about 20 mM. In yet other instances, the TRIS buffer can be at a concentration of about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM or about 30 mM.

In some instances, MgCl₂ can be at a concentration from about 0.5 mM to about 1.5 mM. In other instances, MgCl₂ can be at a concentration from about 0.6 mM to about 1.4 mM, from about 0.7 mM to about 1.3 mM, from about 0.8 mM to about 1.2 mM, from about 0.9 mM to about 1.1 mM, or about 1.0 mM. In yet other instances, MgCl₂ can be at a concentration of about 0.5 mM, about 0.6 mM, about 0.7 mM, about 0.8 mM, about 0.9 mM, about 1.0 mM, about 1.1 mM, about 1.2 mM, about 1.3 mM, about 1.4 mM or about 1.5 mM.

In some instances, NaCl can be at a concentration from about 100 mM to about 300 mM. In other instances, NaCl can be at a concentration from about 125 mM to about 275 mM, from about 150 mM to about 250 mM, from about 175 mM to about 225 mM, or about 200 mM. In yet other instances, NaCl can be at a concentration of about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM or about 300 mM.

In some instances, Poloxamer 188 can be at a concentration from about 0.001% (w/v) to about 0.01% (w/v). In other instances, Poloxamer 188 can be at a concentration from about 0.002% (w/v) to about 0.009% (w/v), from about 0.003% (w/v) to about 0.008% (w/v), from about 0.004% (w/v) to about 0.007% (w/v), or from about 0.005% (w/v) to about 0.006% (w/v). In yet other instances, the Poloxamer 188 can be at a concentration of about 0.001% (w/v), about 0.002% (w/v), about 0.003% (w/v), about 0.004% (w/v), about 0.005% (w/v), about 0.006% (w/v), about 0.007% (w/v), about 0.008% (w/v), about 0.009% (w/v) or about 0.01% (w/v).

In certain instances, the formulation can include a rAAV herein and (a) about 20 mM TRIS (pH 8.0), (b) about 1 mM MgCl₂, (c) about 200 mM NaCl and (d) about 0.005% (w/v) Poloxamer 188.

In some instances, the effective amount can be a titer between about 10⁹ genome copies (GC)/kg to about 10¹⁴ GC/kg (e.g., about 10⁹ GC/kg, about 10¹⁰ GC/kg, about 10¹¹ GC/kg, about 10¹² GC/kg, about 10¹³ GC/kg, or about 10¹⁴ GC/kg). In some instances, the individual is administered a high titer (e.g., >10¹² GC/kg of rAAV) by injection to the CSF space, especially via ICM.

In other instances, the effective amount can be a dose ranging from about 1×10¹² vg to about 1×10¹⁵ vg or about 1×10¹³ vg to about 7×10¹⁴ vg. In other instances, the dose can be about 3.5×10¹³ vg, about 7.0×10¹³ vg or about 1.4×10¹⁴ vg. In yet other instances, the dose can be about 1×10¹⁴ vg, about 2.0×10¹⁴ vg, or about 4.0×10¹⁴ vg. Alternatively, the dose can be about 2×10¹³ vg, about 3×10¹³ vg, about 4×10¹³ vg, about 5×10¹³ vg, about 6×10¹³ vg, about 7×10¹³ vg, about 8×10¹³ vg, about 9×10¹³ vg, about 1×10¹⁴ vg, or about 2×10¹⁴ vg. In certain instances, the dose is 7.0×10¹³ vg or 1.4×10¹⁴ vg.

The pharmaceutical composition can be administered by any route including, for example, intra-arterial, intradermal, intramuscular, intrathecal, intravenous (IV), intraventricular, parenteral, subcutaneous (SC) or transdermal. Specifically contemplated routes are IV administration (e.g., systemic intravenous injection), and/or direct administration to an affected site (e.g., intracisternal magna (ICM) injection, intracerebroventricular (ICV) injection) or combinations thereof.

Generally, the most appropriate route of administration will depend upon a variety of factors including, but not limited to, the nature of the agent (e.g., its stability in the environment of its administration and/or intended target) and/or the condition of the individual (e.g., whether the subject is able to tolerate oral administration). In some instances, the synthetic nucleic acids, rAAV or pharmaceutical compositions are suitable for administration to the CNS of an individual by, for example, intrathecal, ICM, ICV or combinations thereof.

Kits

In some instances, synthetic nucleic acids (i.e., an expression construct or a vector) or rAAVs herein can be included in a kit that includes the synthetic nucleic acid or rAAV and instructions for its use. In other instances, the kit includes the synthetic nucleic acid or rAAV and a package insert containing instructions for use of the kit and/or any component thereof. In yet other instances, the kit comprises, in a suitable container or other means for containing, the synthetic nucleic acid or rAAV, one or more controls, and various buffers, reagents, enzymes and other standard ingredients well known in the art. In some instances, the container comprises at least one vial, well, test tube, flask, bottle, syringe, or other container means, into which the synthetic nucleic acid, rAAV or other therapeutic oligonucleotide is placed, and in some instances, suitably aliquoted. In those instances where an additional component is provided, the kit includes additional containers into which this component is placed. The kits can also include a means for containing the synthetic nucleic acid or rAAV and any other reagent in close confinement for commercial sale. Such containers may include injection or blow-molded plastic containers into which the desired vials are retained. Containers and/or kits can include labeling with instructions for use and/or warnings.

In some instances, the kit includes the synthetic nucleic acid or rAAV and a pharmaceutically acceptable carrier, or a pharmaceutical composition including the synthetic nucleic acid, rAAV or other therapeutic oligonucleotide and instructions for treating or delaying progression of a neurodegenerative disease in an individual in need thereof.

In some instances, the kit includes the synthetic nucleic acid or rAAV and a pharmaceutically acceptable carrier or a pharmaceutical composition comprising the synthetic nucleic acid or rAAV and instructions for administering the synthetic nucleic acid or rAAV or pharmaceutical composition.

Methods

Methods of Making

Methods of making rAAVs are described, for example, in Samulski et al. (1989) J Virol. 63:3822-3828 and Wright (2009) Hum. Gene Ther. 20:698-706. In some instances, the rAAV can be produced in a Baculovirus vector expression system (BEVS). Production of rAAVs using BEVS are described, for example, in Urabe et al. (2002) Hum. Gene Ther. 13:1935-1943, Smith et al. (2009) Mol. Ther. 17:1888-1896, as well as U.S. Pat. Nos. 8,945,918 and 9,879,282, and Intl. Patent Application Publication Nos. WO 2017/184879 and WO 2022/082017. Alternatively, the rAAV can be produced in human embryonic kidney (e.g., HEK293) cells (see, e.g., Intl. Patent Application Publication Nos. WO 2020/210689 and WO 2022/035900). However, the rAAV can be produced using any suitable method (e.g., using recombinant rep and cap genes).

Methods of Treatment and Uses

The nucleic acids, expression constructs, vectors, rAAV or pharmaceutical compositions described herein can be used for treating a neurological disease such as, for example, AD, ALS, ALSP, cognitive deficit, FTD, memory loss, NHD, spinal cord injury, traumatic brain injury, or multiple sclerosis.

The methods can include the steps described herein, and these maybe be, but not necessarily, carried out in the sequence as described. Other sequences, however, also are conceivable. Moreover, individual or multiple steps bay be carried out either in parallel and/or overlapping in time and/or individually or in multiply repeated steps. Furthermore, the methods may include additional, unspecified steps.

Here, the synthetic nucleic acids, rAAV or pharmaceutical compositions including the same may be used in a method to treat TREM2-associated diseases and disorders, where such method includes at least a step of administering to an individual in need of such treatment an effective amount of a synthetic nucleic acid, rAAV or a pharmaceutical composition including the same.

In some instances, the synthetic nucleic acid, rAVV or pharmaceutical composition is administered via an IV injection. In other instances, the synthetic nucleic acid, rAVV or pharmaceutical composition is administered via an ICM injection of the individual.

When a rAAV, the effective amount can be a titer between about 10⁹ genome copies (GC)/kg to about 10¹⁴ GC/kg (e.g., about 10⁹ GC/kg, about 10¹⁰ GC/kg, about 10¹¹ GC/kg, about 10¹² GC/kg, about 10¹³ GC/kg, or about 10¹⁴ GC/kg). In some instances, the individual is administered a high titer (e.g., >10¹² GC/kg of rAAV) by injection to the CSF space, especially via ICM.

In other instances, the effective amount can be a dose ranging from about 1×10¹² vg to about 1×10¹⁵ vg or about 1×10¹³ vg to about 7×10¹⁴ vg. In other instances, the dose can be about 3.5×10¹³ vg, about 7.0×10¹³ vg or about 1.4×10¹⁴ vg. In yet other instances, the dose can be about 1×10¹⁴ vg, about 2.0×10¹⁴ vg, or about 4.0×10¹⁴ vg. Alternatively, the dose can be about 2×10¹³ vg, about 3×10¹³ vg, about 4×10¹³ vg, about 5×10¹³ vg, about 6×10¹³ vg, about 7×10¹³ vg, about 8×10¹³ vg, about 9×10¹³ vg, about 1×10¹⁴ vg, or about 2×10¹⁴ vg. In certain instances, the dose is 7.0×10¹³ vg or 1.4×10¹⁴ vg.

In some instances, the rAAV or composition including the same can be administered to a subject once or multiple times (e.g., 2, 3, 4, 5, 6, 7, 8, 9, 10, 20 or more).

In some instances, the individual has or is suspected of having a disease or disorder associated with TREM2, especially AD, ALSP or NHD.

In some instances, the individual is between the ages of about 1 month old to about 10 years old (e.g., about 1 month, 2 months, 3 months, 4, months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years, 5 years, 6 years, 7 years, 8 years, 9 years, 10 years, or any age therebetween). In other instances, the individual is between about 10 years old to about 20 years old (e.g., about 10 years, 11 years, 12 years, 13 years, 14 years, 15 years, 16 years, 17 years, 18 years, 19 years, 20 years, or any age therebetween). In other instances, the individual is older than 20 years old (e.g., about 21 years, 22 years, 23 years, 24 years, 25 years, 26 years, 27 years, 28 years, 29 years, 30 years, or any age therebetween), older than 30 years old (e.g., about 31 years, 32 years, 33 years, 34 years, 35 years, 36 years, 37 years, 38 years, 39 years, 40 years, or any age therebetween), older than 40 years old (e.g., about 41 years, 42 years, 43 years, 44 years, 45 years, 46 years, 47 years, 48 years, 49 years, 50 years, or any age therebetween), or even older than 50 years old (e.g., about 51 years, 52 years, 53 years, 54 years, 55 years, 56 years, 57 years, 58 years, 59 years, 60 years, 70 years, 80 years, 90 years, or any age therebetween).

In some instances, the individual has a pathogenic CSF IR mutation.

In some instances, the method also can include a step of measuring TREM2 in plasma and/or CSF and/or urine from the individual and comparing an obtained value to an earlier-obtained comparable value or to a control value to assess effectiveness of the method. In some instances, the TREM2 is soluble TREM2 (sTREM2).

In other instances, the method also can include a step of measuring CSF1R in plasma and/or CSF and/or urine from the individual and comparing an obtained value to an earlier-obtained comparable value or to a control value to assess effectiveness of the method. In some instances, the CSF1R is soluble CSF1R (sCSF1R).

In other instances, the method also can include a step of measuring NfL in plasma and/or CSF and/or urine from the individual and comparing an obtained value to an earlier-obtained comparable value or to a control value to assess effectiveness of the method.

In other instances, the method also can include a step of measuring Chitl in plasma and/or CSF and/or urine from the individual and comparing an obtained value to an earlier-obtained comparable value or to a control value to assess effectiveness of the method.

In other instances, the method also can include a step of measuring GM-CSF in plasma and/or CSF and/or urine from the individual and comparing an obtained value to an earlier-obtained comparable value or to a control value to assess effectiveness of the method.

Uses

A rAAV herein or pharmaceutical composition including the same can be used, or adapted for use, to treat an individual (e.g., a human) having or suspected of having a disease or disorder associated with TREM2, such as AD, ALSP or NHD. As such, the rAAV or pharmaceutical composition including the same is provided for use, or adapted for use, to treat an individual having or suspected of having a disease or disorder associated with TREM2, such as AD, ALSP or NDH. Also, the rAAV or pharmaceutical composition including the same is provided for use, or adaptable for use, in the manufacture of a medicament or a pharmaceutical composition for treating a disease or disorder associated with TREM2, such as AD, ALSP or NHD.

Also described are nucleic acids, vectors, rAAVs or pharmaceutical compositions for use in therapy. Furthermore, nucleic acids, vectors, rAAVs or pharmaceutical compositions are described herein for use in the treatment of a neurological disease such as, for example, AD, ALS, ALSP, cognitive deficit, FTD, memory loss, NHD, spinal cord injury, traumatic brain injury or multiple sclerosis.

Also described are use of nucleic acids, vectors, rAAVs or pharmaceutical compositions in the manufacture of a medicament for the treatment of a neurological disease such as, for example, AD, ALS, ALSP, cognitive deficit, FTD, memory loss, NHD, spinal cord injury, traumatic brain injury or multiple sclerosis.

### EXAMPLES

The following non-limiting examples are offered for purposes of illustration, not limitation.

### Example 1: Generating rAAV

Methods: rAAV vectors expressing various TREM2-encoding transgenes were generated using cells, such as HEK293 cells or 519 insect cells (see, e.g., Intl. Patent Application Nos. WO 2008/024988, 2017/184879 and WO 2022/082017). The ITR sequences flank an expression construct having a promoter/enhancer element for the transgene, a 3′ poly A signal, and posttranslational signals such as the WPRE element.

The rAAV vectors were encapsidated in either AAV6TM capsid protein (SEQ ID NO:19) or AAV9 capsid protein (SEQ ID NO:20).

In Vitro Studies

### Example 2: In Vitro Transducing of a Human Microglial Cell Line with TREM-2 rAAV

Methods: A HMC3 microglial cell line was transduced with a rAAV vector having a TREM2 transgene (SEQ ID NO:3) encapsidated in AAV6TM capsid protein (SEQ ID NO:19) or in AAV9 capsid protein (SEQ ID NO:20) at a range of MOIs. 72 hr post-transduction, supernatant was collected for protein and cells were lysed for RNA.

TREM2 mRNA was quantified using qRT-PCR and normalized to GAPDH. TREM2 protein was quantified using a MSD assay.

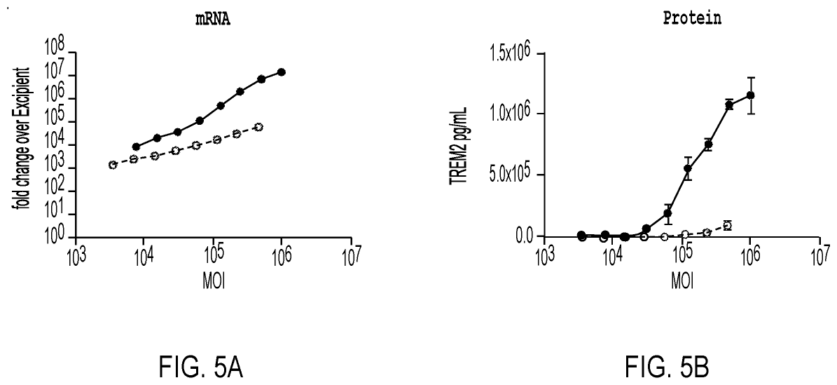

Results: FIGS. 5A-5B show that TREM2 mRNA expression (FIG. 5A) and protein expression (FIG. 5B) were observed with both rAAV; however, rAAV vector encapsidated in AAV6TM capsid protein performed better than the same rAAV vector encapsidated in AAV9 capsid protein.

### Example 3: In Vitro Transducing of a Human Microglial Cell Line with Alternative TREM-2 rAAV

Methods: HMC3 cells were transduced with a first rAAV vector having a TREM2 transgene (SEQ ID NO:25) or a second rAAV vector having a TREM2 transgene (SEQ ID NO:26) encapsidated in AAV6TM capsid protein (SEQ ID NO:19) over a range of MOI from 1.09×10⁵ to 3.50×10⁶ vg/cell. 3 days post-infection, supernatant was collected for ELISA.

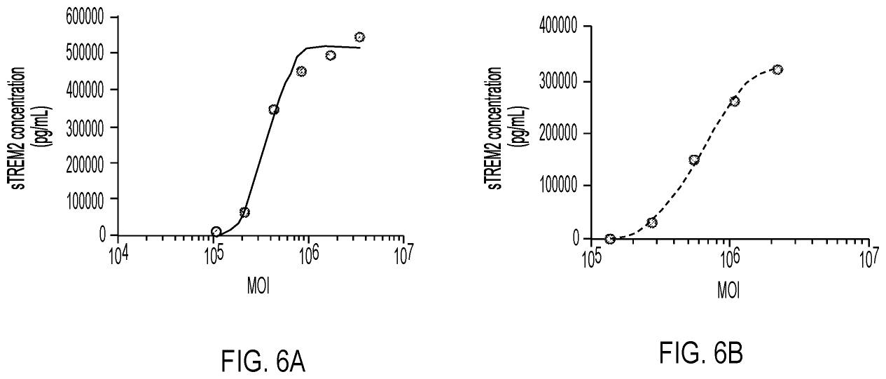

Results: Both rAAV effectively transduced HMC3 cells in vitro, resulting in a robust, dose-dependent expression and secretion of TREM2 (FIGS. 6A-6B, where FIG. 6A is the first rAAV vector and FIG. 6B is the second rAAV vector). In contrast, a minimal level of TREM2 (78 pg/mL) was detected in the negative control group treated with excipient alone.

### Example 4: Determining In Vitro Potency of TREM2 in Human Induced Pluripotent

Stem Cell (iPSC)-Derived Microglia Cells

Methods: Human iPSC-derived microglia either were treated with excipient (negative control) or were transduced with a rAAV vector having a TREM2 transgene (SEQ ID NO:26) encapsidated in AAV6TM capsid protein (SEQ ID NO:19) at a MOI ranging from 2.0×10³ to 2.0×10⁶ vg/cell. 7 days post-transduction, cells were lysed for ELISA or were processed for RNA. All iPSC-derived cells were obtained from FujiFilm Cellular Dynamics, Inc. (Madison, WI).

Results: By Day 7, rAAV transduction resulted in robust, dose-dependent expression of TREM2 that was detected in groups treated at 2.0×10⁵ and 2.0×10⁶ vg/cell of about 1000 pg/mL and 1250 pg/mL, respectively. In contrast, TREM2 was detected from excipient-treated iPSC-derived microglia at a concentration of about 625 pg/mL. Furthermore, rAAV transduction resulted in a dose-dependent increase in mRNA expression of TREM2 at all dose groups, while no TREM2 mRNA was detected from excipient-treated microglia.

### Example 5: Improving Cell Viability with a TREM2 rAAV in a Pharmacological CSF1R-Inhibition Assay in Human iPSC-Derived Microglia Cells

Methods: The iPSC-derived microglia as described in Example 4 either were treated with excipient (negative control) or were transduced with a rAAV vector having a TREM2 transgene (SEQ ID NO:26) encapsidated in AAV6TM capsid protein (SEQ ID NO:19) at a MOI of 2.0×10³ vg/cell. 7 days post-transduction, 150 nM or 200 nM of PLX3397 in 0.1% DMSO was added to the cell culture media and incubated for additional 8 hr at 37° C. and 5% CO₂. After this period of incubation, cells were assayed for viability using a colorimetric kit (Abcam).

Results: PLX treatment caused cytotoxicity and reduced the total number of microglial cells (i.e., about 80% cell death); whereas rAAV transduction significantly reduced the PLX-induced toxicity to 50% as compared to control levels.

In Vivo Studies

### Example 6: In Vivo Rodent Studies of AAV Capsid Proteins

Methods: 36 one-month old 5×FAD mice were allocated into 3 groups (n=12/group) and treated with the same rAAVs as in Example 2 or excipient (20 mM Tris pH 8.0, 200 mM NaCl and 1 mM MgCl₂+0.001% Pluronic F68)) via ICV injection (10 μl-5 μl bilateral per mouse) to determine efficacy. A group (n=12) of non-transgenic, age-matched littermates (i.e., WT) were similar injected with excipient as a control. Treatment doses were 5.68×10¹⁰ vg/animal or 1.8×10¹¹ vg/animal. 5 months post-ICV injection, the presence of vg was assessed by ddPCR.

In addition, efficacy endpoints including protein levels, amyloid-β levels (both biochemical and immunohistochemical) and inflammation were examined. TREM2 protein and amyloid-β levels were measured using a MSD assay. Iba⁺ cells were measured by immunohistochemistry.

In vivo blood collection: In vivo blood was collected by mandibular bleeding from the facial vein/artery plexus without anesthesia before treatment start, and at 2, 3, 4 and 5 months of age (5 time points). Collected blood was then transferred into serum gel clotting activator microtube. After incubation for at least 20 min (60 min maximum) at room temperature, serum was prepared from the samples by centrifugation (10000×g, 5 min, room temperature). After centrifugation, serum was frozen on dry ice and stored at −80° C.

Tissue sampling: At 6 months, the mice were terminally anesthetized by i.p. injection of Pentobarbital (600 mg/kg) and CSF, blood, brain, several organs (gonads, kidney, heart, liver, lung and spleen) and spinal cord were collected for biochemical, immunochemical and/or histological analysis.

Immunohistology: For each incubation a uniform systematic random set of 5 sections per mouse from all animals per group was selected (one section each from levels 2, 4, 6, 8, 10). All sections were counterstained with the nuclear dye DAPI. Binding of primary antibodies was visualized using highly cross-absorbed secondary antibodies.

Imaging: Whole slide scans of the stained sections were recorded on a Zeiss automatic microscope AxioScan Z1 with high aperture lenses, equipped with a Zeiss Axiocam 506 mono and a Hitachi 3CCD HV-F202SCL camera and Zeiss ZEN 3.3 software.

Sample Preparation—Homogenization: Hippocampus and somatosensory cortex from all animals were homogenized in 14 volumes PBS at 55 Hz for 50 sec using UPHO bead mill (Geneye) and 3 aliquots were generated (30 μL for RNA/DNA isolation, 50 μL for soluble insoluble protein isolation and rest).

Sample Preparation—DNA and RNA Isolation: DNA and RNA were isolated from hippocampus and somatosensory cortex from all animals using the Ambion TriZOL kit from a 30 μL PBS aliquot. The amount and quality of total extracted RNA was evaluated by UV-VIS spectrometry using a NanoDrop 1000 spectrophotometer. RNA (1 μg of each sample) was reverse transcribed by using the iScript gDNA Clear cDNA Synthesis Kit.

Amyloid-β40 and Amyloid-β42 Sample Preparation and Measurement (soluble and insoluble): A second aliquot of 50 μl of hippocampus and cortex samples was substituted with the same volume of 2×THB buffer (2×THB; 500 mM Sucrose, 2 mM EDTA, 2 mM EGTA, 40 mM Tris pH 7.4) including 1× protease inhibitor (Calbiochem) vortexed and incubated for 15 min on ice. The THB homogenate was then processed for extraction of soluble and deposited proteins from brain homogenates for analysis of amyloid-β40 and amyloid-β42. For extraction of non-plaque associated proteins, 50 μl THB homogenate were mixed with 1-part DEA solution (0.4% DEA, 100 mM NaCl). The mixture was centrifuged for 120 min at 20,000×g, 4° C. The supernatant was neutralized with 1/10 of the volume 0.5 M Tris-HCl, pH 6.8 and vortexed briefly. Aliquots were stored at −80° C. For extraction of deposited proteins, 30 μL THB homogenate were mixed with 2.2 parts cold FA, sonicated for 30 sec on ice and centrifuged for 120 min at 20,000×g, 4° C. The supernatant was mixed with 19 parts FA Neutralization Solution (1M Tris, 0.5 M Na₂HPO₄, 0.05% NaN₃). Aliquots were stored at −80° C. Soluble and insoluble fraction of hippocampus and somatosensory cortex samples from all animals were then analyzed by immunosorbent assays from Mesoscale Discovery (Amyloid-β40 Peptide (6E10) Kit and Amyloid-β42 Peptide (6E10) Kit) according to the instructions of the manufacturer. Amyloid-β levels in study samples were evaluated in comparison to calibration curves provided in the kit and are expressed as pg/mg brain.

Statistics: Statistical analysis was performed in GraphPad Prism 9. Data was tested for normality using the Kolmogorow-Smirnow-Test. If normal distribution was confirmed, differences between groups were tested with the one or two-way ANOVA/Mixed-effects analysis followed by Bonferroni, Dunnett's or multiple comparisons test. If data was not normally distributed, differences between groups were tested with Kruskal-Wallis-Test, followed by Dunn's post hoc test for multiple comparisons. The 5×FAD-excipient group was used as reference group. Data are presented as mean+ or +/−SEM.

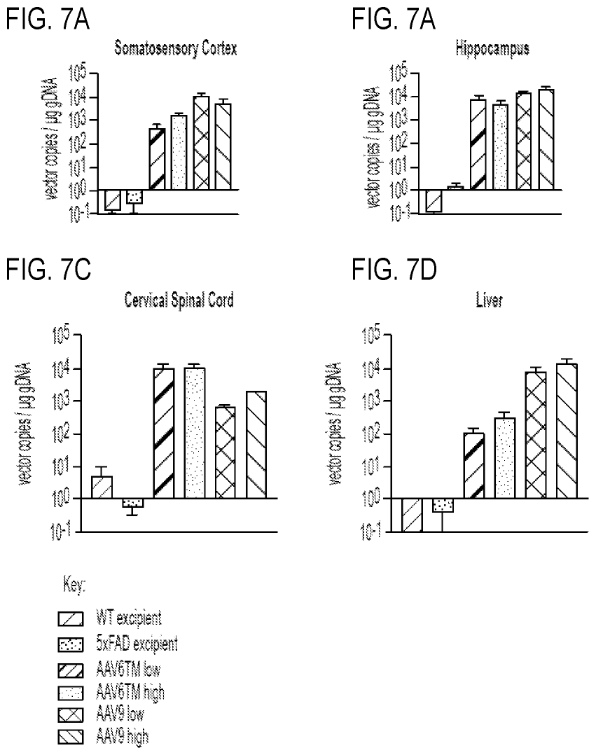

Results: FIGS. 7A-7D show both capsid proteins resulted in broad biodistribution in the cortex, hippocampus, cervical spinal cord and liver.

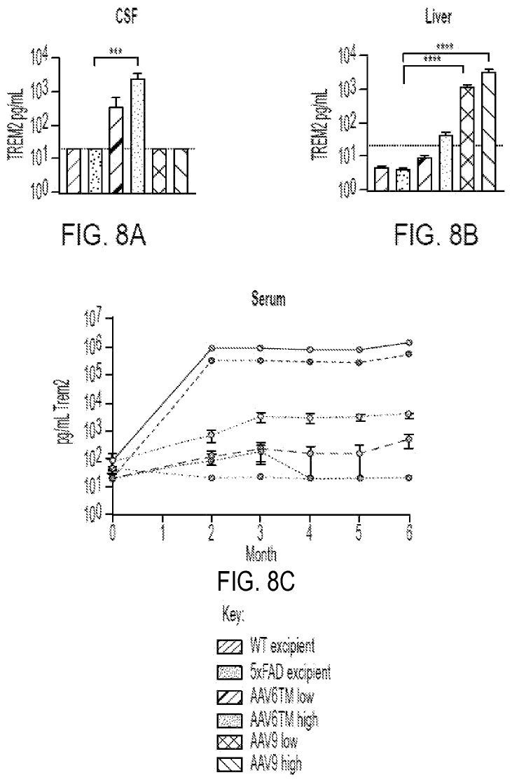

FIGS. 8A-8C show both capsid proteins resulted in TREM2 protein levels in CSF, liver and serum, where the AAV6TM capsid protein showed higher TREM2 protein levels in the CSF than the AAV9 capsid protein.

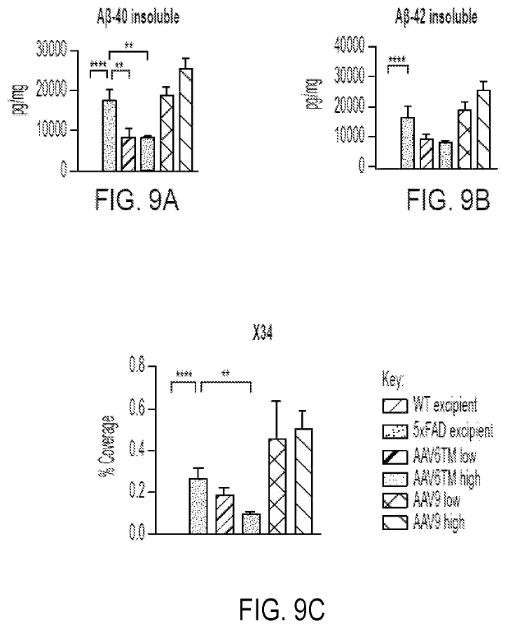

FIGS. 9A-9C show that the rAAV vector encapsidated in AAV6TM capsid protein significantly decreased amyloid-β in the hippocampus and cortex whereas the rAAV vector encapsidated in AAV9 capsid protein did not decrease amyloid-β in the hippocampus and cortex.

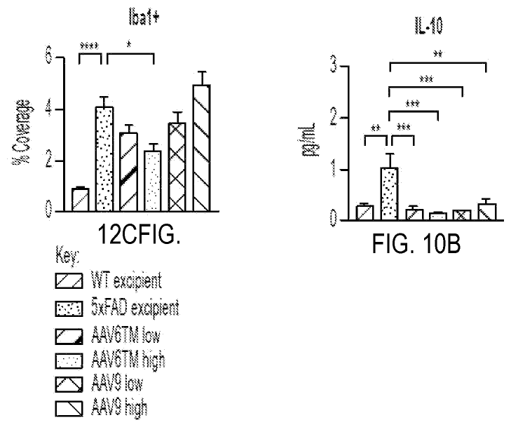

FIGS. 10A-10B show that rAAV vector decreased inflammatory markers in cortex regardless of capsid protein.

Overall, rAAV vector dosing resulted in broad biodistribution and dose-dependent increases in TREM2 protein levels for both capsids; however, significant reduction of cortical disease burden was observed only in mice treated with AAV6TM capsid protein.

### Example 7: In Vivo Rodent Studies of TREM2 rAAV

Methods: Excipient or 2 different doses of one or the other of the TREM2 rAAVs of Example 3 were administered to 1-month old WT C57BL/6 mice by ICV injection (n=10/group). The rAAV were injected at a dose of either 5.68×10¹⁰ vg (1.42×10¹¹ vg/g brain) or 1.8×10¹¹ vg (4.49×10¹¹ vg/g brain), respectively. Vector particles per gram brain weight were calculated based on an adult mouse brain weight of 400 mg.

CNS tissues and liver were collected to analyze biodistribution by ddPCR. CSF, liver, and serum were collected to analyze TREM2 expression.

Biodistribution was determined by measuring vg presence using ddPCR (with >50 vg/1 μg gDNA defined as positive).

Results: Mice that received rAAV were positive for vg in the cortex indicating that ICV administration successfully results in comparable transduction in the brain. There was no significant effect of vector design on biodistribution (all ps>0.05). ICV administration also resulted in vg presence in the spinal cord and liver.

TREM2 expression was measured in the CSF and serum using MSD. ICV injection of rAAV increased TREM2 in the CSF at both doses. These data indicate that there is no difference in TREM2 expression levels generated by either rAAV, consistent with the biodistribution data. Additionally, ICV injection of rAAV significantly increased levels of TREM2 in the blood at 1.8×10¹¹ vg. A dose-dependent effect in serum was observed when comparing the doses.

### Example 8: Assessing In Vivo Efficacy of TREM2 rAAV in a CSF1R-Inhibition Mouse Model for CRL

Methods: Excipient or 2 different doses of a TREM2 rAAV of Example 3 (i.e., a rAAV vector of SEQ ID NO:26 encapsidated in AAV6TM capsid protein (SEQ ID NO:19)) were administered to 1-month old WT C57BL/6 mice by ICV injection (n=10). The rAAV was injected at a dose of either 1.8×10¹⁰ vg (4.49×10¹⁰ vg/g brain) or 1.8×10¹¹ vg (4.49×10¹¹ vg/g brain), respectively. Vector particles per g brain weight was based on an adult mouse brain weight of 400 mg. At 8 weeks of age (i.e., 4 weeks post-ICV injection), 1 excipient-treated group and both dose-treated groups were fed with PLX-containing mouse chow. Mice were fed with PLX-chow at 185 mg/kg for 1 week and then sacrificed at 9 weeks of age (i.e., 5 weeks post-ICV injection).

Biodistribution was determined by measuring vg presence using ddPCR (with >50 vg/1 μg gDNA defined as positive).

Results: Mice that received rAAV were positive for vg in all the brain tissues tested, indicating that ICV administration successfully resulted in transduction in the brain. ICV administration also resulted in vg presence in the spinal cord, with lower levels of transduction observed in the liver. Reduced biodistribution in the liver was reflected in the lower TREM2 levels in this tissue.

Inhibition of CSF1R by PLX treatment was apparent at both the mRNA and protein level in mice. There was a statistically significant improvement in CSF1R expression at the mRNA level in mice treated with both doses of rAAV+PLX as compared to mice treated with excipient+PLX. The level of sCSF1R fragment in the CSF of animals treated with both doses of rAAV+PLX exhibited a positive trending pattern toward excipient levels. Thus, rAAV treatment ameliorates the negative effect of PLX in the CSF1R-inhibition model.

Moreover, dosing of mice with excipient+PLX resulted in almost complete depletion of microglia throughout the brain. In fact, there was a statistically significant increase in the number of IBA1⁺ microglia in mice that were treated with either dose of rAAV+PLX as compared to mice treated with excipient+PLX. This observation was further confirmed by analysis of AIF1 mRNA expression level, a gene that encodes IBA1, using qRT-PCR. Furthermore, a significant therapeutic benefit of rAAV was evident in higher mRNA levels of AIF1, in mice treated with either dose+PLX as compared to treatment group with excipient+PLX.

Furthermore, PLX treatment decreased expression of genes associated with homeostatic microglia, such as P2RY12 and HEXB, which are known to be stably expressed in the microglia in the homeostatic state. In mice treated with rAAV+PLX, a significantly higher expression of both P2RY12 and HEXB was observed.

Consistent with the PLX model-effect that was observed in universal and homeostatic genes, disease associated microglia (DAM) genes (e.g., CCL12, CD48, CD68, LYZ1 and PTPRC) also showed a significant decrease in expression in the excipient+PLX treatment group. In contrast, expression of these genes was fully, or in some cases partially, elevated by rAAV, in most cases to normal levels as compared to excipient-only treated animals.

### Example 9: TREM2 rAAV Dose-Ranging in a CSF1R-Inhibition Mouse Model for CRL

Methods: Excipient or a TREM2 rAAV of Example 3 (i.e., a rAAV vector of SEQ ID NO:26 encapsidated in AAV6TM capsid protein (SEQ ID NO:19)) were administered to 4-week-old WT C57BL/6 mice by ICV injection (n=16/group). TREM2 rAAV was injected at the following 3 doses: 1.33×10¹¹ vg (3.33×10¹¹ vg/g brain), 1.8×10¹⁰ vg (4.49×10¹⁰ vg/g brain) or 2.44×10⁹ vg (6.10×10⁹ vg/g brain). Vector particles per g brain weight was based on an adult mouse brain weight of 400 mg.

At 8 weeks of age (i.e., 4 weeks post-ICV injection), 1 excipient-treated group and both 1.33×10¹¹ vg- and 2.44×10⁹-treated groups were treated with PLX3397. Mice were fed with PLX3397-chow at 185 mg/kg for 1 week and then sacrificed at 9 weeks of age (i.e., 5 weeks post-ICV injection).

Biodistribution, TREM2 expression levels, microglial panel gene expression, and safety through histopathology was assessed. Biodistribution was determined using ddPCR, which was developed in alignment with the FDA's guidance “Long-Term Follow-up After Administration of Human Gene Therapy Products” (2020; with limit of quantification of <50 copies/μg of gDNA). TREM2 expression was measured in the CSF using an MSD assay (as above, sTREM2 was used as a proxy for protein production within the tissues).

Results: All mice that received TREM2 rAAV were positive for vg in all brain regions tested, indicating that ICV administration successfully resulted in widespread transduction in the brain. Additionally, ICV administration of TREM2 rAAV resulted in reduced vector transduction in the liver as compared to brain.

ICV administration of TREM2 rAAV robustly increased levels of TREM2 in the CSF at all 3 doses. Lower biodistribution of TREM2 rAAV in peripheral tissues was reflected in lower TREM2 levels in the liver, as well as lower TREM2 levels in serum. A dose-dependent effect was observed in the serum when comparing the 1.33×10¹¹ vg and 2.44×10⁹ vg doses.

Out of the 3 doses tested in this study, the 2.44×10⁹ vg dose exhibited no efficacious benefit in almost any of the microglial genes tested (i.e., a subtherapeutic dose). The other two doses exhibited significant improvements in microglial gene expression profile in TREM2 rAAV+PLX-treated mice as compared to excipient+PLX-treated mice, in almost 90% of genes with model effect.

Taken together, the 1.33×10¹¹ vg and 1.8×10¹⁰ vg doses suppressed PLX-induced microglial damage in the brainstem of the CSF1R-inhibition mouse model. This effect also was observed in different brain regions, including the hippocampus, cortex and cerebellum.

### Example 10: In Vivo Non-Human Primate (NHP) Studies of TREM2 rAAV

Methods: 2-4-year-old female cynomolgus monkeys were administered a rAAV vector having a TREM2 transgene of SEQ ID NO:3 encapsidated in AAV6TM capsid protein (SEQ ID NO:19) or excipient via ICM injection. One-month post-ICM injection, the presence of vg, TREM2 and safety were assessed.

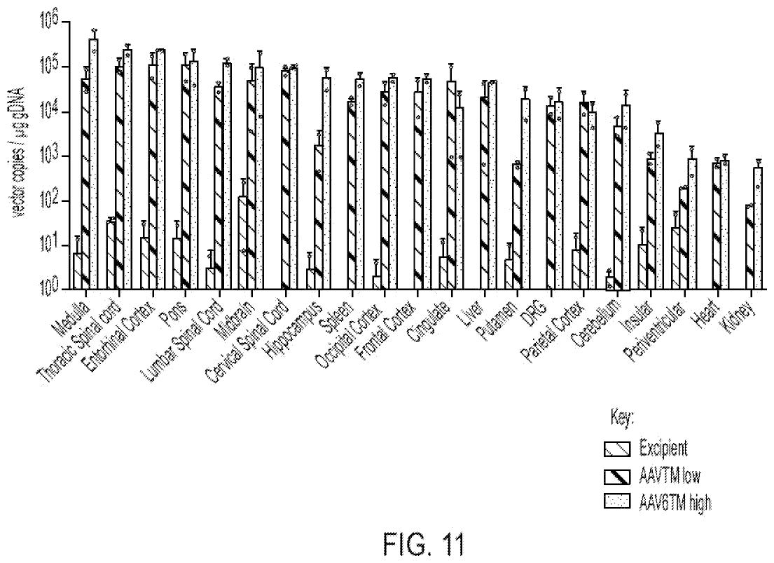

Results: FIG. 11 shows that AAV6TM capsid protein resulted in broad biodistribution in NHPs.

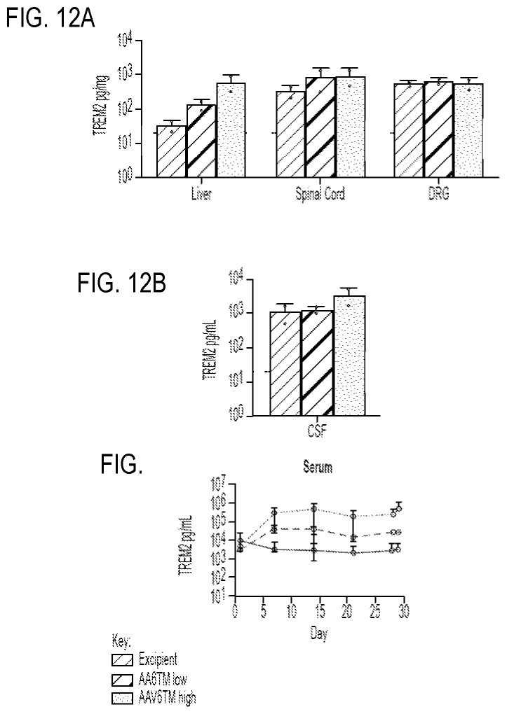

FIGS. 12A-12C show that the AAV6TM capsid protein resulted in increased TREM2 protein levels in liver, spinal cord, CSF and serum.

Overall, in the NHP safety studies, all animals survived and there was no test article-related variations in mortality, clinical signs, body weights/body weight gains, body temperature and neurological parameters in either group dosed via a single injection into the ICM. No findings from examination of general attitude, behavior, motor function, proprioception and postural reaction. Clinical pathology revealed minimal changes in hematology, coagulation and clinical chemistry. Histopathology findings were limited and comparable to AAV9 capsid protein in previous NHP studies. In the high dose, microscopic findings of minimal to mild mononuclear cell infiltration in the brain and DRG were noted.

Both AAV9 capsid protein and AAV6TM capsid proteins transduced microglial cells in vitro with the rAAV vector encoding TREM2; however, AAV6TM capsid protein drove higher TREM2 mRNA and protein expression as compared to AAV9 capsid protein. In vivo, both rAAV demonstrated broad biodistribution and dose-dependent increases in TREM2 protein levels. Additionally, AAV6TM capsid protein exhibited comparable biodistribution to AAV9 capsid protein in key brain regions in the rodent studies while showing substantially less liver tropism.

### Example 11: In Vivo NHP Studies of TREM2 rAAV

Methods: Excipient or a TREM2 rAAV of Example 3 (a rAAV vector of SEQ ID NO:25 encapsidated in AAV6TM capsid protein (SEQ ID NO:19)) or vehicle were administered to female cynomolgus monkeys via ICM (n=2/group). TREM2 rAAV was injected at the following 2 doses: 3.32×10¹² vg (4.49×10¹⁰ vg/g brain) or 1.05×10¹³ vg (1.42×10₁₁ vg/g brain). The NHPs were sacrificed 29 days after injection to detect potential early toxicity of TREM2 rAAV, with the expectation of capturing potential toxicity at a time in which biodistribution throughout the brain and peripheral organs was expected to be near its maximum.

Biodistribution, TREM2 expression levels, microglial panel gene expression, and safety through histopathology was assessed. Biodistribution was determined using ddPCR as described in Example 9. TREM2 expression was measured in the CSF using an MSD assay (as above, sTREM2 was used as a proxy for protein production within the tissues).

Results: All NHPs survived to Day 29. There were no test article-related variations observed in mortality, clinical signs, body weights/body weight gains, body temperature or neurological signs in either group dosed with a single injection of TREM2 rAAV into the ICM at the 3.32×10¹² dose or 1.05×10¹³ total vg dose.

Minimal changes in hematology and coagulation parameters were observed in NHPs treated with the 1.05×10¹³ vg dose. These changes consisted of an increased neutrophil count at Days 15 and 29, with increased fibrinogen levels at Day 15 as compared to baseline values, indicating a potential acute phase response.

There were no TREM2 rAAV-related macroscopic observations. Test article-related microscopic observations included minimal to mild mononuclear cell infiltration in the brain in all NHPS administered≥3.32×10¹² vg. The minimal to mild mononuclear cell infiltration was perivascular in distribution and also present in the pia. The mononuclear cells were primarily lymphocytes, and the distribution and severity were greater in the animals receiving the 3.32×10¹² vg dose.

Given that the only treatment-related findings were minimal increases in neutrophils and fibrinogen and minimal to mild mononuclear infiltration of the brain and ganglia, ICM injection of either dose of TREM2 rAAV was judged to be well-tolerated in NHPs.

As for biodistribution, all tissues tested were positive in NHPs administered TREM2 rAAV, indicating widespread distribution throughout the CNS and periphery.

In the CSF, TREM2 rAAV administration increased TREM2 in the 1.05×10¹³ vg dose. In contrast, a dose-dependent effect of the 2 doses on TREM2 was observed in serum and liver.

Taken together, the results showed broad distribution throughout the brain, comparable to the levels shown to be efficacious in mouse models. This transduction leads to the elevation of TREM2 in the brain. Furthermore, all NHPs survived, and postmortem pathology analysis indicated minimal test article-related increases in neutrophils and fibrinogen, as well as minimal to mild mononuclear infiltration of the brain and ganglia. Consequently, the TREM2 rAAV demonstrated a generally favorable safety profile in NHPs.

### Example 12: In Vivo NHP Studies of TREM2 rAAV

Methods: Excipient or a TREM2 rAAV of Example 3 (a rAAV vector of SEQ ID NO:26 encapsidated in AAV6TM capsid protein (SEQ ID NO:19)) or vehicle will be administered to cynomolgus monkeys (male and female) via ICM (n=6/group). TREM2 rAAV will be injected at one of the following 3 doses: 3.32×10¹² vg (4.49×10¹⁰ vg/g brain), 1.05×10¹³ vg (1.42×10¹¹ vg/g brain) or 3.32×10¹³ vg (4.49×10¹¹ vg/g brain). The NHPs will be followed for 29 days or 26 weeks after injection to assess tolerability and safety.

This study also will assess peak vector distribution in the brain at about 4 weeks post-administration, as well as post-peak assessments at 183 days post-administration. Control NHPs will receive an ICM administration of the same volume of formulation buffer excipient including 20 mM Tris (pH 8.0), 1 mM MgCl₂, and 200 mM NaCl containing 0.005% (w/v) Poloxamer 188. All NHPs will be screened for AAV6 neutralizing antibodies (NAb) with a 1:20 titer cut-off.

### SEQUENCE LISTING

The following nucleic and/or amino acid sequences are referred to in the disclosure above and are provided below for reference.

SEQ ID NO: 1-human TREM2 prctcin iscfcrm 1 (230 aa)
MEPLRLLILLFVTELSGAHNTTVFQGVAGQSLQVSCPYDSMKHWGRRKAWCRQLGE
KGPCQRVVSTHNLWLLSFLRRWNGSTAITDDTLGGTLTITLRNLQPHDAGLYQCQSL
HGSEADTLRKVLVEVLADPLDHRDAGDLWFPGESESFEDAHVEHSISRSLLEGEIPFP
PTSILLLLACIFLIKILAASALWAAAWHGQKPGTHPPSELDCGHDPGYQLQTLPGLRD
T
SEQ ID NO: 2-human TREM2 prctcin iscfcrm 2 (219 aa)
MEPLRLLILLFVTELSGAHNTTVFQGVAGQSLQVSCPYDSMKHWGRRKAWCRQLGE
KGPCQR VVSTHNLWLLSFLRRWNGSTAITDDTLGGTLTITLRNLQPHDAGLYQCQSL
HGSEADTLRKVLVEVLADPLDHRDAGDLWFPGESESFEDAHVEHSISRAERHVKED
DGRKSPGEVPPGTSPACILATWPPGLLVLLWQETTLPEHCFSWTLEAGTG
SEQ ID NO: 3-Artificial scqucncc (TREM2 transgcnc 1; 690 nt)
atggagcccctgcgcctgctgatcctgctgttcgtgaccgagctgagcggcgcccacaacaccaccgtgttccagggcgtggccgg
ccagagcctgcaggtgagctgcccctacgacagcatgaagcactggggccgccgcaaggcctggtgccgccagctgggcgagaa
gggcccctgccagcgcgtggtgagcacccacaacctgtggctgctgagcttcctgcgccgctggaacggcagcaccgccatcacc
gacgacaccctgggcggcaccctgaccatcaccctgcgcaacctgcagccccacgacgccggcctgtaccagtgccagagcctgc
acggcagcgaggccgacaccctgcgcaaggtgctggtggaggtgctggccgaccccctggaccaccgcgacgccggcgacctgt
ggttccccggcgagagcgagagcttcgaggacgcccacgtggagcacagcatcagccgcagcctgctggagggcgagatcccctt
cccccccaccagcatcctgctgctgctggcctgcatcttcctgatcaagatcctggccgccagcgccctgtgggccgccgcctggca
cggccagaagcccggcacccacccccccaggagctggactgcggccacgaccccggctaccagctgcagaccctgcccggcct
gcgcgacacc
SEQ ID NO: 4-Artificial scqucncc (TREM2 transgcnc 2; 690 nt)
atggagcccctgaggctgctcatcctgctgtttgtgacagaactgtctggagcccacaacaccacagtgttccagggagttgctggcca
gtctctgcaagtgtcttgcccctatgacagcatgaagcactggggaaggaggaaggcttggtgtaggcagctgggagagaaaggac
cttgccagagggtggtgagcacacacaacctgtggctgctgagcttcctcagaaggtggaatggctctacagccatcacagatgaca
ccctgggtggcaccctcaccatcaccttaaggaacctgcagcctcatgatgctggcctgtaccaatgccagagcctgcatggctctga
ggctgataccctcaggaaggtgttggtggaggtgctggctgatcctctggatcacagggatgctggagacctgtggttcccaggaga
gtctgagagctttgaggatgcccatgtggagcacagcatcagcaggtctcttctggagggagagatccccttccctcccacaagcatc
ctgttgctgcttgcctgcatcttcctgatcaagatccttgctgcttctgctctttgggctgctgcctggcatggccagaaacctggaacaca
tcctccctctgaactggactgtggccatgaccctggctaccagttgcaaaccttgcctggcttgagggacacc
SEQ ID NO: 5-Artificial scqucncc (TREM2 transgcnc 3; 690 nt)
atggagcccctgaggctgctgatcctgctgtttgtgacagaactgtctggagcccacaacaccacagtgttccagggagttgctggcc
agtctctgcaagtgtcttgcccctatgacagcatgaagcactggggaaggaggaaggcttggtgtaggcagctgggagagaaagga
ccttgccagagggtggtgagcacacacaacctgtggctgctgagcttcctcagaaggtggaatggctctacagccatcacagatgac
accctgggtggcaccctcaccatcaccctgaggaacctgcagcctcatgatgctggcctgtaccaatgccagagcctgcatggctctg
aggctgataccctcaggaaggtgttggtggaggtgctggctgatcctctggatcacagggatgctggagacctgtggttcccaggaga
gtctgagagctttgaggatgcccatgtggagcacagcatcagcaggtctcttctggagggagagatccccttccctcccacaagcatc
ctgttgctgcttgcctgcatcttcctcatcaagatccttgctgcttctgctctttgggctgctgcctggcatggccagaaacctggaacaca
tcctccctctgaactggactgtggccatgaccctggctaccagttgcaaaccttgcctggcttgagggacacc
SEQ ID NO: 6-Artificial scqucncc (TREM 2 transgcnc 4; 690 nt)
atggagcccctgagactgctcatcctgctgtttgtgacagaactgtctggggcccacaacaccacagtgttccagggggtggctggcc
agtccctccaggtgtcctgcccctatgactccatgaagcactgggggagaagaaaggcttggtgtagacagctgggggagaaaggg
ccttgccagagagtggtgtccacacacaacctgtggctgctgtccttcctgagaagatggaatggctccacagccatcacagatgaca
ccctggggggcaccctcaccatcaccctgagaaacctgcagcctcatgatgctggcctgtaccagtgccagtccctgcatggctctga
ggctgataccctgagaaaggtgctggtggaggtgctggctgatcctctggatcacagagatgctggggacctgtggttcccagggga
gtctgagtcctttgaggatgcccatgtggagcactccatctccagatccctgctggagggggagatccccttccctcccacatccatcct
gctgctgctggcctgcatcttcctcatcaagatcctggctgcttctgctctgtgggctgctgcctggcatggccagaaacctgggacaca
tcctccctctgaactggactgtggccatgaccctggctaccagctgcagaccctgcctggcctgagagacacc
SEQ ID NO: 7-Chickcn β-actin prcmctcr (283 nt)
catggtcgaggtgagccccacgttctgcttcactctccccatctcccccccctccccacccccaattttgtatttatttattttttaattatt
ttgtgcagcgatgggggcggggggggggggggggcgcgcgccaggcggggggggcggggcgaggggggggggggcgag
gcggagaggtgcggcggcagccaatcagagcggcgcgctccgaaagtttccttttatggcgaggcggcggcggcggcggccctat
aaaaagcgaagcgcgcggcgggcg
SEQ ID NO: 8-CD68 prcmctcr (715 nt)
gatatcaaactgcctgtttgggcttctcatttcttacctccccttccctctcccacctgctactgggtgcatctctgctccccccttccccagc
agatggttacctttgggctgttgctttcttgtcaccatctgagttctcagacgctggaaagccatgttctcggctctgtgaatgacaatgctg
actggagtgctgcccctctgtaaagggctgggtgtggatggtcacaagcccctcacatgcctcagccaagaggaagtagtacagggg
tcagcccagaggtccaggggaaaggagtggaaaccgatttccccaccaagggaggggcctgtacctcagctgttcccatagcttact
tgccacaactgccaagcaagtttcgctgagtttgacacatggatccctgtggatcaactgccctaggactccgtttgcacccatgtgaca
ctgttgactttgccctgacgaagcagggccaacagtcccctaacttaattacaaaaactaatgactaagagagaggtggctagagctga
ggcccctgagtcaggctgtggggggatcatctccagtacaggaagtgagactttcatttcctcctttccaagagagggctgagggag
cagggttgagcaactggtgcagacagcctagctggactttgggtgaggcggttcagccatatcgaattctgctggggctactggcag
SEQ ID NO: 9-F4/80 prcmctcr (2068 nt)
gaattctttgtttaggtctgtatgccatatttaatagggttatttggttctctggagtctaacttcctgagttctttgtatattttggatatca
gtcctctatcagatgttgggttggtaaagatttttttcccccaatctgtgagttgctgttttgtcatattgacagtctcctttgctttacagaa
gctttttaatttcatgaggtcccatttgtcaattgttgatcttagagcctgagccattggtgttctgtacaggacaaattttcccctgtgccaa
tatgttcgaggttcttccccattttcttttctattagactcagtgtatctggttttatgtggaggtccttgatccactcggacttgagctttgt
acaaggagataagaatggatcagtttgtatttttctacatcctaactgccagttgaactagcaacatttgttgaaaatgtttttttttcttccc
tcactggatgactttgacttctttgtcaaagatcaagtgaccataggtgtgtaggttcacttttgggtcttcaattttattccattgatctacc
tttctgtcattgtatcaataccatgaaggttttttaaaaaaaaatttttttaatcatattgctctgcagtacagcttgaggtcagggatggtga
ttcccccagaggctctttgattgttgagaacggtttttgctatcctgggttttttgttattccagatgaatttgagttgggattttgataggga
atacactgaatttgtagattgcttttggcaagatggccatctttccatcttctgaggtctttgatttctttcttcagagacttgaagttcttgt
catacagatctttcaattgcttggttagagtcacaccaagatattttatattatttgtgactattgtgaagggtgtcatttctctaatttattt
ctcagcccgtttatcctttgagtagaggaaaactactgatttgtttgaattaattttatatctagccactaagctgaagttgtttatcagctg
taggagttctctggtggattattttttagggttatttatgtattctaccacatcatctgcaaatagtgatatcttgacttcctcctttctaatt
tgtatccctttgacctccttttgttgcctaattgttctgggtagaactttgagtactatattaaatgataggggaaaagtggacagccttggg
tgtggtgtgtgtggtatggtgtggtgtgtgtatggtgatgatgcatgtggtgtgtatggtgtggtgtatgtggtgtggtgtgtggtggtggtg
ataatgatgatggtggtctctctgtctctgtctgtctctttctatctctgcctctctctctctgtctctctctgcccccctctgtctccctct
gtctgtctgtctgtctctttggtgtgatgtatgtggtgtgtgtgtattctacaaggttgacatgatgacagaatttaattttcttagcagcaa
gctcatggatcctggtgataaatgcagcatgactttactgaaaaggctttgtgatcttgaagagtggattgacttcactgtcggcagcacatg
caatctcacttgtttggtgtaatgaaagaagagaatgagaggtggaagggggatggtaatgttgaaaaaaagaatggtacagaggaaactgag
gttggagagagatggggtagatggtaagagatggagaaagagggaaggaaatggagagaaagacagagagacagagagagacacacagagaga
cacacagagacagagaggaagggaaagggaaagagaaaggaagaggaagagggggaggggaaggggaaggggaaggggaagggagagggagaa
atgtggacactagccagatttaagggagaaattagggggttgccagtctgtccacctctgatggtggcaactcagcagaaagctgctgggctca
gtctggctttgttgagcaaccctgactccaccccttttcttccccacaaagcaagcttttaaagggaaggctttcttcattgaatgactgcca
cagtacgatg
SEQ ID NO: 10-Cytcmcgalcvirus cnhanccr (352 nt)
aatagtaatcaattacggggtcattagttcatagcccatatatggagttccgcgttacataacttacggtaaatggcccgcctggctgacc
gcccaacgacccccgcccattgacgtcaataatgacgtatgttcccatagtaacgccaatagggactttccattgacgtcaatgggtgg
agtatttacggtaaactgcccacttggcagtacatcaagtgtatcatatgccaagtacgccccctattgacgtcaat gacggtaaatggc
ccgcctggcattatgcccagtacatgaccttatgggactttcctacttggcagtacatctacgtattagtcatcgctattac
SEQ ID NO: 11-Wccdchuck hcpatitis virus pcst-transcripticnal rcgulatcry clcmcnt
(594 nt)
ttatcgataatcaacctctggattacaaaatttgtgaaagattgactggtattcttaactatgttgctccttttacgctatgtggatacgctg
ctttaatgcctttgtatcatgctattgcttcccgtatggctttcattttctcctccttgtataaatcctggttgctgtctctttatgaggagt
tgtggcccgttgtcaggcaacgtggcgtggtgtgcactgtgtttgctgacgcaacccccactggttggggcattgccaccacctgtcagctcc
tttccgggactttcgctttccccctccctattgccacggcggaactcatcgccgcctgccttgcccgctgctggacaggggctcggctgttggg
cactgacaattccgtggtgttgtcggggaaatcatcgtcctttccttggctgctcgcctgtgttgccacctggattctgcgcgggacgtcc
ttctgctacgtcccttcggccctcaatccagcggaccttccttcccgcggcctgctgccggctctgcggcctcttccgcgtcttcgccttc
gccctcagacgagtcggatctccctttgggccgcctccc
SEQ ID NO: 12-Bcvinc Grcwth Hcrmcnc PclyA Signal Tail (235 nt)
cgactagagctcgctgatcagcctcgactgtgccttctagttgccagccatctgttgtttgcccctcccccgtgccttccttgaccctgga
aggtgccactcccactgtcctttcctaataaaatgaggaaattgcatcgcattgtctgagtaggtgtcattctattctggggggggggtg
gggcaggacagcaagggggaggattgggaagacaatagcaggcatgctgggga
SEQ ID NO: 13-wild-typc AAV2 ITR (145 nt)
aggaacccctagtgatggagttggccactccctctctgcgcgctcgctcgctcactgaggccgggcgaccaaaggtcgcccgacgc
ccgggctttgcccgggcggcctcagtgagcgagcgagcgcgcagagagggagtggccaa
SEQ ID NO: 14-mcdificd AAV2 ITR (141 nt)
cctgcaggcagctgcgcgctcgctcgctcactgaggccgcccgggcaaagcccgggcgtcgggcgacctttggtcgcccggcctc
agtgagcgagcgagcgcgcagagagggagtggccaactccatcactaggggttcct
SEQ ID NO: 15-Artificial scqucncc (TRY rcgicn; 60 nt)
agctctgggtatttaagcccgagtgagcacgcagggtctccattttgaagcgggaggtta
SEQ ID NO: 16-Artificial scqucncc (Stuffcr scqucncc 1; 1229 nt)
gcagtcctcgcatgcctgagtgccctgttctgcactcctctcctccccaccctgcccatattcttcacaccctccttccctccccagtatca
aaacctaccctcagactctttctgcatgtggacatgtaaggggctggtggactctggttgcattggaagggaaggagtgctaacagtgg
catcccaggcactagctggcagttgggggaagctttggggggcactggcactggtaatagcctctgaaattataagccactaattatga
gcccctacagttataaaggaggaaagaacctgaggatgttcatctgcatctttggggcactctctcccctgctctgaaggtccccttgtcc
tcagctcttgatgggaggtgaggagcgtagagatttccgttgctgagttgaggggaattgacacaccaaatttttgtacacaattgcttctt
cccgctgaggaaagcgcaccttttgctcccccagagcactggacaaacctgggcaagaggagagacggtgccaaatggagtcttgtt
cctgcagcttcttagatggtggtcagggggaaggcggggttctgaggcatggatgggaggtggtcagatgggagagggccacggc
ccatctggtctctctagttccaaaaggcaagccaccaacttcccaatcctatctttcaagcctctgtcacagtaatggttgaagatggcag
gcaagggaggaccaagagagaagtcaaagcagggggctctgggggctgccccagcaacaggctggtgctctaagcccatctccc
ccgccctaaggagggttcccaaaatagcagcctcatgtctcccccaaaatatctccgagacgggtccttcctgaaaggggaacaaag
ccacagaaatagggaagctggaagttaaaggtcaggaaagtcggtaccaatgtgggcggctgcagagcaagcaagagtggcggg
gcagggagagccagccccaggccgagaggagaggtcccagtcccaaatgatggcaaagagatgtgcagaacagaactggaggg
ggttttaagaccaagtgcctccagaatagacccagagaaggtcagttacttctccaaggaggcccagcaagagcaacagtgagaaaa
ccaaacccaggtcccaggtttcctggttcccaatttcccacaaacacatgctgtgccatccgctcccaacttgtataagaacctaagg
SEQ ID NO: 17-Artificial scqucncc (Stuffcr scqucncc 2; 1229 nt)
gcagtcctcacttacctgagtgccctgttctgcactcctctcctccccaccctgcccatattcttcacaccctccttccctccccagtatcaa
aacctaccctcagactctttctgctgtgagactgataaggggctggtggactctggttgcattggaagggaaggagtgcttacagtggc
atcccaggcactagctggcagttgggggaagctttggggggcactggcactggtaatagcctctgaaattataa gcctctcatttgaag
cccctacagttataaaggaggaaagaacctgaggtgattcatctgcatctttggggcactctctcccctgctctgaaggtccccttgtcct
cagctcttgtgaggaggtcaggagcatagagatttccattgctgagttgaggggaacttacacaccaaatttttgtacacaattgcttcttc
ccactgaggaaagcacaccttttgctcccccagagcactggacaaacctgggcaagaggagagacagtgcctgaaagagtcttgttc
ctgcagcttcttagtgagtggtcagggggaaggcagggttctgaggctgagtgaggaggtggtcagtgaggagagggccacagcc
catctggtctctctagttccaaaaggcaagccaccaacttcccaatcctatctttcaagcctctgtcacagttgaagttgaagtgagcagg
caagggaggaccaagagagaagtcaaagcagggggctctgggggctgccccagcaacaggctggtgctctaagcccatctcccc
caccctaaggagggttcccaaaatagcagcctctcatctcccccaaaatatctccaagacaggtccttcctgaaaggggaacaaagcc
acagaaatagggaagctggaagttaaaggtcaggaaagtcagtacctgtgaaggcagctgcagagcaagcaagagtggcagggca
gggagagccagccccaggccaagaggagaggtcccagtccctgtgaaaagcaaagagtgtgacagaacagaactggagggggtt
ttaagaccaagtgcctccagaatagacccagagaaggtcagttacttctccaaggaggcccagcaagagcaacagtgagaaaacca
aacccaggtcccaggtttcctggttcccaatttcccacaaacacttactgtgccatccactcccaacttgtataagaacctaagg
SEQ ID NO: 18-Artificial scqucncc (Stuffcr scqucncc 3; 1254 nt)
attgactgaattcccgtgcggaccggcagtcctcacttacctgagtgccctgttctgcactcctctcctccccaccctgcccatattcttca
caccctccttccctccccagtatcaaaacctaccctcagactctttctgctgtgagactgataaggggctggtggactctggttgcattgg
aagggaaggagtgcttacagtggcatcccaggcactagctggcagttgggggaagctttggggggcactggcactggtaatagcct
ctgaaattataagcctctcatttgaagcccctacagttataaaggaggaaagaacctgaggtgattcatctgcatctttggggcactctctc
ccctgctctgaaggtccccttgtcctcagctcttgtgaggaggtcaggagcatagagatttccattgctgagttgaggggaacttacaca
ccaaatttttgtacacaattgcttcttcccactgaggaaagcacaccttttgctcccccagagcactggacaaacctgggcaagaggag
agacagtgcctgaaagagtcttgttcctgcagcttcttagtgagtggtcagggggaaggcagggttctgaggctgagtgaggaggtgg
tcagtgaggagagggccacagcccatctggtctctctagttccaaaaggcaagccaccaacttcccaatcctatctttcaagcctctgtc
acagttgaagttgaagtgagcaggcaagggaggaccaagagagaagtcaaagcagggggctctgggggctgccccagcaacag
gctggtgctctaagcccatctcccccaccctaaggagggttcccaaaatagcagcctctcatctcccccaaaatatctccaagacaggt
ccttcctgaaaggggaacaaagccacagaaatagggaagctggaagttaaaggtcaggaaagtcagtacctgtgaaggcagctgca
gagcaagcaagagtggcagggcagggagagccagccccaggccaagaggagaggtcccagtccctgtgaaaagcaaagagtgt
gacagaacagaactggagggggttttaagaccaagtgcctccagaatagacccagagaaggtcagttacttctccaaggaggccca
gcaagagcaacagtgagaaaaccaaacccaggtcccaggtttcctggttcccaatttcccacaaacacttactgtgccatccactccca
acttgtataagaacctaagg
SEQ ID NO: 19-AAV6TM capsid prctcin (736 aa)
MAADGYLPDWLEDNLSEGIREWWDLKPGAPKPKANQQKQDDGRGLVLPGYKYLG
PFNGLDKGEPVNAADAAALEHDKAYDQQLKAGDNPYLRYNHADAEFQERLQEDTS
FGGNLGRAVFQAKKRVLEPFGLVEEGAKTAPGKKRPVEQSPQEPDSSSGIGKTGQQP
AKKRLNFGQTGDSESVPDPQPLGEPPATPAAVGPTTMASGGGAPMADNNEGADGV
GNASGNWHCDSTWLGDRVITTSTRTWALPTYNNHLYKQISSASTGASNDNHYFGYS
TPWGYFDFNRFHCHFSPRDWQRLINNNWGFRPKRLNFKLFNIQVKEVTTNDGVTTIA
NNLTSTVQVFSDSEYQLPYVLGSAHQGCLPPFPADVFMIPQYGYLTLNNGSQAVGRS
SFYCLEYFPSQMLRTGNNFTFSYTFEDVPFHSSYAHSQSLDRLMNPLIDQYLYYLNRT
QNQSGSAQNKDLLFSRGSPAGMSVQPKNWLPGPCYRQQRVSKVKTDNNNSNFTWT
GASKYNLNGRESIINPGTAMASHKDDKDKFFPMSGVMIFGKESAGASNTALDNVMIT
DEEEIKATNPVATERFGTVAVNLQSSSTDPATGDVHVMGALPGMVWQDRDVYLQG
PIWAKIPHTDGHFHPSPLMGGFGLKHPPPQILIKNTPVPANPPAEFSATKFASFITQYST
GQVSVEIEWELQKENSKRWNPEVQYTSNFAKSANVDFTVDNNGLYTEPRPIGTRFLT
RPL
SEQ ID NO: 20-AVV9 capsid prctcin (736 aa)
MAADGYLPDWLEDNLSEGIREWWALKPGAPQPKANQQHQDNARGLVLPGYKYLG
PGNGLDKGEPVNAADAAALEHDKAYDQQLKAGDNPYLKYNHADAEFQERLKEDTS
FGGNLGRAVFQAKKRLLEPLGLVEEAAKTAPGKKRPVEQSPQEPDSSAGIGKSGAQP
AKKRLNFGQTGDTESVPDPQPIGEPPAAPSGVGSLTMASGGGAPVADNNEGADGVG
SSSGNWHCDSQWLGDRVITTSTRTWALPTYNNHLYKQISNSTSGGSSNDNAYFGYST
PWGYFDFNRFHCHFSPRDWQRLINNNWGFRPKRLNFKLFNIQVKEVTDNNGVKTIA
NNLTSTVQVFTDSDYQLPYVLGSAHEGCLPPFPADVFMIPQYGYLTLNDGSQAVGRS
SFYCLEYFPSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSEFAWPGA
SSWALNGRNSLMNPGPAMASHKEGEDRFFPLSGSLIFGKQGTGRDNVDADKVMITN
EEEIKTTNPVATESYGQVATNHQSAQAQAQTGWVQNQGILPGMVWQDRDVYLQGP
IWAKIPHTDGNFHPSPLMGGFGMKHPPPQILIKNTPVPADPPTAFNKDKLNSFITQYST
GQVSVEIEWELQKENSKRWNPEIQYTSNYYKSNNVEFAVNTEGVYSEPRPIGTRYLT
RNL
SEQ ID NO: 21-Artificial scqucncc (Vcctcr 1; 8305 nt)
cctgcaggcagctgcgcgctcgctcgctcactgaggccgcccgggcaaagcccgggcgtcgggcgacctttggtcgcccggcctc
agtgagcgagcgagcgcgcagagagggagtggccaactccatcactaggggttcctgcggccgcacgcgtgttactaggttctaga
accggtgacgtctcccatggtgaagcttggatctgagatatcaaactgcctgtttgggcttctcatttcttacctccccttccctctcccacc
tgctactgggtgcatctctgctccccccttccccagcagatggttacctttgggctgttgctttcttgtcaccatctgagttctcagacgctg
gaaagccatgttctcggctctgtgaatgacaatgctgactggagtgctgcccctctgtaaagggctgggtgtggatggtcacaagcccc
tcacatgcctcagccaagaggaagtagtacaggggtcagcccagaggtccaggggaaaggagtggaaaccgatttccccaccaag
ggaggggcctgtacctcagctgttcccatagcttacttgccacaactgccaagcaagtttcgctgagtttgacacatggatccctgtgga
tcaactgccctaggactccgtttgcacccatgtgacactgttgactttgccctgacgaagcagggccaacagtcccctaacttaattaca
aaaactaatgactaagagagaggtggctagagctgaggcccctgagtcaggctgtgggtgggatcatctccagtacaggaagtgag
actttcatttcctcctttccaagagagggctgagggagcagggttgagcaactggtgcagacagcctagctggactttgggtgaggcg
gttcagccatatcgaattctgctggggctactggcaggtaaggaggaaggaggctgaggggagggggcccctgggagggagcctg
ccctgggttgctaaccatctcctctctgccaaaagtccggaaagccaccatggagcccctgcgcctgctgatcctgctgttcgtgaccg
agctgagcggcgcccacaacaccaccgtgttccagggcgtggccggccagagcctgcaggtgagctgcccctacgacagcatgaa
gcactggggccgccgcaaggcctggtgccgccagctgggcgagaagggcccctgccagcgcgtggtgagcacccacaacctgtg
gctgctgagcttcctgcgccgctggaacggcagcaccgccatcaccgacgacaccctgggcggcaccctgaccatcaccctgcgc
aacctgcagccccacgacgccggcctgtaccagtgccagagcctgcacggcagcgaggccgacaccctgcgcaaggtgctggtg
gaggtgctggccgaccccctggaccaccgcgacgccggcgacctgtggttccccggcgagagcgagagcttcgaggacgcccac
gtggagcacagcatcagccgcagcctgctggagggcgagatccccttcccccccaccagcatcctgctgctgctggcctgcatcttc
ctgatcaagatcctggccgccagcgccctgtgggccgccgcctggcacggccagaagcccggcacccacccccccagcgagctg
gactgcggccacgaccccggctaccagctgcagaccctgcccggcctgcgcgacacctgacaattgttaattaagtttaaaccctcga
ggccgcaagcttatcgataatcaacctctggattacaaaatttgtgaaagattgactggtattcttaactatgttgctccttttacgctatgtg
gatacgctgctttaatgcctttgtatcatgctattgcttcccgtatggctttcattttctcctccttgtataaatcctggttgctgtctcttt
atgaggagttgtggcccgttgtcaggcaacgtggcgtggtgtgcactgtgtttgctgacgcaacccccactggttggggcattgccaccacctg
tcagctcctttccgggactttcgctttccccctccctattgccacggcggaactcatcgccgcctgccttgcccgctgctggacaggggc
tcggctgttgggcactgacaattccgtggtgttgtcggggaaatcatcgtcctttccttggctgctcgcctgtgttgccacctggattctgc
gcgggacgtccttctgctacgtcccttcggccctcaatccagcggaccttccttcccgcggcctgctgccggctctgcggcctcttccg
cgtcttcgccttcgccctcagacgagtcggatctccctttgggccgcctccccgcatcgataccgtcgactagagctcgctgatcagcc
tcgactgtgccttctagttgccagccatctgttgtttgcccctcccccgtgccttccttgaccctggaaggtgccactcccactgtcctttcc
taataaaatgaggaaattgcatcgcattgtctgagtaggtgtcattctattctggggggtggggggggcaggacagcaagggggagg
attgggaagacaatagcaggcatgctggggagagatccacgataacaaacagcttttttggggtgaacatattgactgaattcccgtgc
ggaccggcagtcctcacttacctgagtgccctgttctgcactcctctcctccccaccctgcccatattcttcacaccctccttccctcccca
gtatcaaaacctaccctcagactctttctgctgtgagactgataaggggctggtggactctggttgcattggaagggaaggagtgcttac
agtggcatcccaggcactagctggcagttgggggaagctttggggggcactggcactggtaatagcctctgaaattataagcctctcat
ttgaagcccctacagttataaaggaggaaagaacctgaggtgattcatctgcatctttggggcactctctcccctgctctgaaggtcccct
tgtcctcagctcttgtgaggaggtcaggagcatagagatttccattgctgagttgaggggaacttacacaccaaatttttgtacacaattg
cttcttcccactgaggaaagcacaccttttgctcccccagagcactggacaaacctgggcaagaggagagacagtgcctgaaagagt
cttgttcctgcagcttcttagtgagtggtcagggggaaggcagggttctgaggctgagtgaggaggtggtcagtgaggagagggcca
cagcccatctggtctctctagttccaaaaggcaagccaccaacttcccaatcctatctttcaagcctctgtcacagttgaagttgaagtga
gcaggcaagggaggaccaagagagaagtcaaagcagggggctctgggggctgccccagcaacaggctggtgctctaagcccatc
tcccccaccctaaggagggttcccaaaatagcagcctctcatctcccccaaaatatctccaagacaggtccttcctgaaaggggaaca
aagccacagaaatagggaagctggaagttaaaggtcaggaaagtcagtacctgtgaaggcagctgcagagcaagcaagagtggca
gggcagggagagccagccccaggccaagaggagaggtcccagtccctgtgaaaagcaaagagtgtgacagaacagaactggag
ggggttttaagaccaagtgcctccagaatagacccagagaaggtcagttacttctccaaggaggcccagcaagagcaacagtgaga
aaaccaaacccaggtcccaggtttcctggttcccaatttcccacaaacacttactgtgccatccactcccaacttgtataagaacctaagg
cggaccgagcggccgcaggaacccctagtgatggagttggccactccctctctgcgcgctcgctcgctcactgaggccgggcgacc
aaaggtcgcccgacgcccgggctttgcccgggcggcctcagtgagcgagcgagcgcgcagctgcctgcaggcatgcaagctgta
gccaaccactagaactatagctagagtcctgggcgaacaaacgatgctcgccttccagaaaaccgaggatgcgaaccacttcatccg
gggtcagcaccaccggcaagcgccgcgacggccgaggtcttccgatctcctgaagccagggcagatccgtgcacagcaccttgcc
gtagaagaacagcaaggccgccaatgcctgacgatgcgtggagaccgaaaccttgcgctcgttcgccagccaggacagaaatgcct
cgacttcgctgctgcccaaggttgccgggtgacgcacaccgtggaaacggatgaaggcacgaacccagttgacataagcctgttcg
gttcgtaaactgtaatgcaagtagcgtatgcgctcacgcaactggtccagaaccttgaccgaacgcagcggtggtaacggcgcagtg
gcggttttcatggcttgttatgactgtttttttgtacagtctatgcctcgggcatccaagcagcaagcgcgttacgccgtgggtcgatgtttg
atgttatggagcagcaacgatgttacgcagcagcaacgatgttacgcagcagggcagtcgccctaaaacaaagttaggtggctcaag
tatgggcatcattcgcacatgtaggctcggacctgaccaagtcaaatccatgcgggctgctcttgatcttttcggtcgtgagttcggaga
cgtagccacctactcccaacatcagccggactccgattacctcgggaacttgctccgtagtaagacattcatcgcgcttgctgccttcga
ccaagaagcggttgttggcgctctcgcggcttacgttctgcccaggtttgagcagccgcgtagtgagatctatatctatgatctcgcagt
ctccggcgagcaccggaggcagggcattgccaccgcgctcatcaatctcctcaagcatgaggccaacgcgcttggtgcttatgtgat
ctacgtgcaagcagattacggtgacgatcccgcagtggctctctatacaaagttgggcatacgggaagaagtgatgcactttgatatcg
acccaagtaccgccacctaacaattcgttcaagccgagatcggcttcccggccgcggagttgttcggtaaattgtcacaacgccgcga
atatagtctttaccatgcccttggccacgcccctctttaatacgacgggcaatttgcacttcagaaaatgaagagtttgctttagccataac
aaaagtccagtatgctttttcacagcataactggactgatttcagtttacaactattctgtctagtttaagactttattgtcatagtttagat
ctattttgttcagtttaagactttattgtccgcccacacccgcttacgcagggcatccatttattactcaaccgtaaccgattttgccaggtt
acgcggctggtctgcggtgtgaaataccgcacagatgcgtaaggagaaaataccgcatcaggcgctcttccgcttcctcgctcactgactcg
ctgcgctcggtcgttcggctgcggcgagcggtatcagctcactcaaaggcggtaatacggttatccacagaatcaggggataacgca
ggaaagaacatgtgagcaaaaggccagcaaaaggccaggaaccgtaaaaaggccgcgttgctggcgtttttccataggctccgccc
ccctgacgagcatcacaaaaatcgacgctcaagtcagaggtggcgaaacccgacaggactataaagataccaggcgtttccccctg
gaagctccctcgtgcgctctcctgttccgaccctgccgcttaccggatacctgtccgcctttctcccttcgggaagcgtggcgctttctca
atgctcacgctgtaggtatctcagttcggtgtaggtcgttcgctccaagctgggctgtgtgcacgaaccccccgttcagcccgaccgct
gcgccttatccggtaactatcgtcttgagtccaacccggtaagacacgacttatcgccactggcagcagccactggtaacaggattagc
agagcgaggtatgtaggcggtgctacagagttcttgaagtggtggcctaactacggctacactagaaggacagtatttggtatctgcgc
tctgctgaagccagttaccttcggaaaaagagttggtagctcttgatccggcaaacaaaccaccgctggtagcggtggtttttttgtttgc
aagcagcagattacgcgcagaaaaaaaggatctcaagaagatcctttgatcttttctacggggtctgacgctcagtggaacgaaaactc
acgttaagggattttggtcatgagattatcaaaaaggatcttcacctagatccttttaaattaaaaatgaagttttaaatcaatctaaagtata
tatgagtaaacttggtctgacagttaccaatgcttaatcagtgaggcacctatctcagcgatctgtctatttcgttcatccatagttgcctgac
tccccgtcgtgtagataactacgatacgggagggcttaccatctggccccagtgctgcaatgataccgcgagacccacgctcaccggc
tccagatttatcagcaataaaccagccagccggaagggccgagcgcagaagtggtcctgcaactttatccgcctccatccagtctatta
attgttgccgggaagctagagtaagtagttcgccagttaatagtttgcgcaacgttgttgccattgctacaggcatcgtggtgtcacgctc
gtcgtttggtatggcttcattcagctccggttcccaacgatcaaggcgagttacatgatcccccatgttgtgcaaaaaagcggttagctcc
ttcggtcctccgatcgttgtcagaagtaagttggccgcagtgttatcactcatggttatggcagcactgcataattctcttactgtcatgcca
tccgtaagatgcttttctgtgactggtgagtactcaaccaagtcattctgagaatagtgtatgcggcgaccgagttgctcttgcccggcgt
caatacgggataataccgcgccacatagcagaactttaaaagtgctcatcattggaaaacgttcttcggggcgaaaactctcaaggatc
ttaccgctgttgagatccagttcgatgtaacccactcgtgcacccaactgatcttcagcatcttttactttcaccagcgtttctgggtgagca
aaaacaggaaggcaaaatgccgcaaaaaagggaataagggcgacacggaaatgttgaatactcatactcttcctttttcaatattattga
agcatttatcagggttattgtctcatgagcggatacatatttgaatgtatttagaaaaataaacaaataggggttccgcgcacatttccccg
aaaagtgccacctgaaattgtaaacgttaatattttgttaaaattcgcgttaaatttttgttaaatcagctcattttttaaccaataggccga
aatcggcaaaatcccttataaatcaaaagaatagaccgagatagggttgagtgttgttccagtttggaacaagagtccactattaaagaacgt
ggactccaacgtcaaagggcgaaaaaccgtctatcagggcgatggcccactacgtgaaccatcaccctaatcaagttttttggggtcg
aggtgccgtaaagcactaaatcggaaccctaaagggagcccccgatttagagcttgacggggaaagccggcgaacgtggcgagaa
aggaagggaagaaagcgaaaggagcgggcgctagggcgctggcaagtgtagcggtcacgctgcgcgtaaccaccacacccgcc
gcgcttaatgcgccgctacagggcgcgtccattcgccattcaggctgcaaataagcgttgatattcagtcaattacaaacattaataacg
aagagatgacagaaaaattttcattctgtgacagagaaaaagtagccgaagatgacggtttgtcacatggagttggcaggatgtttgatt
aaaaacataacaggaagaaaaatgccccgctgtgggcggacaaaatagttgggaactgggaggggtggaaatggagtttttaaggat
tatttagggaagagtgacaaaatagatgggaactgggtgtagcgtcgtaagctaatacgaaaattaaaaatgacaaaatagtttggaact
agatttcacttatctggttcggatctcctagagcttacagctt
SEQ ID NO: 22-Artificial scqucncc (Vcctcr 2; 8305 nt)
cattcgccattcaggctgcaaataagcgttgatattcagtcaattacaaacattaataacgaagagatgacagaaaaattttcattctgtga
cagagaaaaagtagccgaagatgacggtttgtcacatggagttggcaggatgtttgattaaaaacataacaggaagaaaaatgccccg
ctgtgggcggacaaaatagttgggaactgggaggggtggaaatggagtttttaaggattatttagggaagagtgacaaaatagatggg
aactgggtgtagcgtcgtaagctaatacgaaaattaaaaatgacaaaatagtttggaactagatttcacttatctggttcggatctcctaga
gcttacagcttcctgcaggcagctgcgcgctcgctcgctcactgaggccgcccgggcaaagcccgggcgtcgggcgacctttggtc
gcccggcctcagtgagcgagcgagcgcgcagagagggagtggccaactccatcactaggggttcctgcggccgcacgcgtgttac
taggttctagaaccggtgacgtctcccatggtgaagcttggatctgagatatcaaactgcctgtttgggcttctcatttcttacctccccttc
cctctcccacctgctactgggtgcatctctgctccccccttccccagcagatggttacctttgggctgttgctttcttgtcaccatctgagttc
tcagacgctggaaagccatgttctcggctctgtgaatgacaatgctgactggagtgctgcccctctgtaaagggctgggtgtggatggt
cacaagcccctcacatgcctcagccaagaggaagtagtacaggggtcagcccagaggtccaggggaaaggagtggaaaccgattt
ccccaccaagggaggggcctgtacctcagctgttcccatagcttacttgccacaactgccaagcaagtttcgctgagtttgacacatgg
atccctgtggatcaactgccctaggactccgtttgcacccatgtgacactgttgactttgccctgacgaagcagggccaacagtccccta
acttaattacaaaaactaatgactaagagagaggtggctagagctgaggcccctgagtcaggctgtgggtgggatcatctccagtaca
ggaagtgagactttcatttcctcctttccaagagagggctgagggagcagggttgagcaactggtgcagacagcctagctggactttg
ggtgaggcggttcagccatatcgaattctgctggggctactggcaggtaaggaggaaggaggctgaggggagggggcccctggga
gggagcctgccctgggttgctaaccatctcctctctgccaaaagtccggaaagccaccatggagcccctgaggctgctcatcctgctg
tttgtgacagaactgtctggagcccacaacaccacagtgttccagggagttgctggccagtctctgcaagtgtcttgcccctatgacagc
atgaagcactggggaaggaggaaggcttggtgtaggcagctgggagagaaaggaccttgccagagggtggtgagcacacacaac
ctgtggctgctgagcttcctcagaaggtggaatggctctacagccatcacagatgacaccctgggtggcaccctcaccatcaccttaag
gaacctgcagcctcatgatgctggcctgtaccaatgccagagcctgcatggctctgaggctgataccctcaggaaggtgttggtggag
gtgctggctgatcctctggatcacagggatgctggagacctgtggttcccaggagagtctgagagctttgaggatgcccatgtggagc
acagcatcagcaggtctcttctggagggagagatccccttccctcccacaagcatcctgttgctgcttgcctgcatcttcctgatcaagat
ccttgctgcttctgctctttgggctgctgcctggcatggccagaaacctggaacacatcctccctctgaactggactgtggccatgaccc
tggctaccagttgcaaaccttgcctggcttgagggacacctgacaattgttaattaagtttaaaccctcgaggccgcaagcttatcgataa
tcaacctctggattacaaaatttgtgaaagattgactggtattcttaactatgttgctccttttacgctatgtggatacgctgctttaatgcc
tttgtatcatgctattgcttcccgtatggctttcattttctcctccttgtataaatcctggttgctgtctctttatgaggagttgtggcccgt
tgtcaggcaacgtggcgtggtgtgcactgtgtttgctgacgcaacccccactggttggggcattgccaccacctgtcagctcctttccggga
ctttcgctttccccctccctattgccacggcggaactcatcgccgcctgccttgcccgctgctggacaggggctcggctgttgggcactgac
aattccgtggtgttgtcggggaaatcatcgtcctttccttggctgctcgcctgtgttgccacctggattctgcgcgggacgtccttctgcta
cgtcccttcggccctcaatccagcggaccttccttcccgcggcctgctgccggctctgcggcctcttccgcgtcttcgccttcgccctca
gacgagtcggatctccctttgggccgcctccccgcatcgataccgtcgactagagctcgctgatcagcctcgactgtgccttctagttgc
cagccatctgttgtttgcccctcccccgtgccttccttgaccctggaaggtgccactcccactgtcctttcctaataaaatgaggaaattgc
atcgcattgtctgagtaggtgtcattctattctggggggtggggggggcaggacagcaagggggaggattgggaagacaatagcag
gcatgctggggagagatccacgataacaaacagcttttttggggtgaacatattgactgaattcccgtgcggaccggcagtcctcactt
acctgagtgccctgttctgcactcctctcctccccaccctgcccatattcttcacaccctccttccctccccagtatcaaaacctaccctca
gactctttctgctgtgagactgataaggggctggtggactctggttgcattggaagggaaggagtgcttacagtggcatcccaggcact
agctggcagttgggggaagctttggggggcactggcactggtaatagcctctgaaattataagcctctcatttgaagcccctacagttat
aaaggaggaaagaacctgaggtgattcatctgcatctttggggcactctctcccctgctctgaaggtccccttgtcctcagctcttgtgag
gaggtcaggagcatagagatttccattgctgagttgaggggaacttacacaccaaatttttgtacacaattgcttcttcccactgaggaaa
gcacaccttttgctcccccagagcactggacaaacctgggcaagaggagagacagtgcctgaaagagtcttgttcctgcagcttctta
gtgagtggtcagggggaaggcagggttctgaggctgagtgaggaggtggtcagtgaggagagggccacagcccatctggtctctct
agttccaaaaggcaagccaccaacttcccaatcctatctttcaagcctctgtcacagttgaagttgaagtgagcaggcaagggaggac
caagagagaagtcaaagcagggggctctgggggctgccccagcaacaggctggtgctctaagcccatctcccccaccctaaggag
ggttcccaaaatagcagcctctcatctcccccaaaatatctccaagacaggtccttcctgaaaggggaacaaagccacagaaataggg
aagctggaagttaaaggtcaggaaagtcagtacctgtgaaggcagctgcagagcaagcaagagtggcagggcagggagagccag
ccccaggccaagaggagaggtcccagtccctgtgaaaagcaaagagtgtgacagaacagaactggagggggttttaagaccaagt
gcctccagaatagacccagagaaggtcagttacttctccaaggaggcccagcaagagcaacagtgagaaaaccaaacccaggtcc
caggtttcctggttcccaatttcccacaaacacttactgtgccatccactcccaacttgtataagaacctaaggcggaccgagcggccgc
aggaacccctagtgatggagttggccactccctctctgcgcgctcgctcgctcactgaggccgggcgaccaaaggtcgcccgacgc
ccgggctttgcccgggcggcctcagtgagcgagcgagcgcgcagctgcctgcaggcatgcaagctgtagccaaccactagaactat
agctagagtcctgggcgaacaaacgatgctcgccttccagaaaaccgaggatgcgaaccacttcatccggggtcagcaccaccggc
aagcgccgcgacggccgaggtcttccgatctcctgaagccagggcagatccgtgcacagcaccttgccgtagaagaacagcaagg
ccgccaatgcctgacgatgcgtggagaccgaaaccttgcgctcgttcgccagccaggacagaaatgcctcgacttcgctgctgccca
aggttgccgggtgacgcacaccgtggaaacggatgaaggcacgaacccagttgacataagcctgttcggttcgtaaactgtaatgca
agtagcgtatgcgctcacgcaactggtccagaaccttgaccgaacgcagcggtggtaacggcgcagtggcggttttcatggcttgttat
gactgtttttttgtacagtctatgcctcgggcatccaagcagcaagcgcgttacgccgtgggtcgatgtttgatgttatggagcagcaacg
atgttacgcagcagcaacgatgttacgcagcagggcagtcgccctaaaacaaagttaggtggctcaagtatgggcatcattcgcacat
gtaggctcggccctgaccaagtcaaatccatgcgggctgctcttgatcttttcggtcgtgagttcggagacgtagccacctactcccaac
atcagccggactccgattacctcgggaacttgctccgtagtaagacattcatcgcgcttgctgccttcgaccaagaagcggttgttggc
gctctcgcggcttacgttctgcccaggtttgagcagccgcgtagtgagatctatatctatgatctcgcagtctccggcgagcaccggag
gcagggcattgccaccgcgctcatcaatctcctcaagcatgaggccaacgcgcttggtgcttatgtgatctacgtgcaagcagattacg
gtgacgatcccgcagtggctctctatacaaagttgggcatacgggaagaagtgatgcactttgatatcgacccaagtaccgccacctaa
caattcgttcaagccgagatcggcttcccggccgcggagttgttcggtaaattgtcacaacgccgcgaatatagtctttaccatgccctt
ggccacgcccctctttaatacgacgggcaatttgcacttcagaaaatgaagagtttgctttagccataacaaaagtccagtatgctttttca
cagcataactggactgatttcagtttacaactattctgtctagtttaagactttattgtcatagtttagatctattttgttcagtttaagact
ttattgtccgcccacacccgcttacgcagggcatccatttattactcaaccgtaaccgattttgccaggttacgcggctggtctgcggtgtg
aaataccgcacagatgcgtaaggagaaaataccgcatcaggcgctcttccgcttcctcgctcactgactcgctgcgctcggtcgttcggctg
cggcgagcggtatcagctcactcaaaggcggtaatacggttatccacagaatcaggggataacgcaggaaagaacatgtgagcaaa
aggccagcaaaaggccaggaaccgtaaaaaggccgcgttgctggcgtttttccataggctccgcccccctgacgagcatcacaaaa
atcgacgctcaagtcagaggtggcgaaacccgacaggactataaagataccaggcgtttccccctggaagctccctcgtgcgctctc
ctgttccgaccctgccgcttaccggatacctgtccgcctttctcccttcgggaagcgtggcgctttctcaatgctcacgctgtaggtatctc
agttcggtgtaggtcgttcgctccaagctgggctgtgtgcacgaaccccccgttcagcccgaccgctgcgccttatccggtaactatcg
tcttgagtccaacccggtaagacacgacttatcgccactggcagcagccactggtaacaggattagcagagcgaggtatgtaggcgg
tgctacagagttcttgaagtggtggcctaactacggctacactagaaggacagtatttggtatctgcgctctgctgaagccagttaccttc
ggaaaaagagttggtagctcttgatccggcaaacaaaccaccgctggtagcggtggtttttttgtttgcaagcagcagattacgcgcag
aaaaaaaggatctcaagaagatcctttgatcttttctacggggtctgacgctcagtggaacgaaaactcacgttaagggattttggtcatg
agattatcaaaaaggatcttcacctagatccttttaaattaaaaatgaagttttaaatcaatctaaagtatatatgagtaaacttggtctgaca
gttaccaatgcttaatcagtgaggcacctatctcagcgatctgtctatttcgttcatccatagttgcctgactccccgtcgtgtagataacta
cgatacgggagggcttaccatctggccccagtgctgcaatgataccgcgagacccacgctcaccggctccagatttatcagcaataaa
ccagccagccggaagggccgagcgcagaagtggtcctgcaactttatccgcctccatccagtctattaattgttgccgggaagctaga
gtaagtagttcgccagttaatagtttgcgcaacgttgttgccattgctacaggcatcgtggtgtcacgctcgtcgtttggtatggcttcattc
agctccggttcccaacgatcaaggcgagttacatgatcccccatgttgtgcaaaaaagcggttagctccttcggtcctccgatcgttgtc
agaagtaagttggccgcagtgttatcactcatggttatggcagcactgcataattctcttactgtcatgccatccgtaagatgcttttctgtg
actggtgagtactcaaccaagtcattctgagaatagtgtatgcggcgaccgagttgctcttgcccggcgtcaatacgggataataccgc
gccacatagcagaactttaaaagtgctcatcattggaaaacgttcttcggggcgaaaactctcaaggatcttaccgctgttgagatccag
ttcgatgtaacccactcgtgcacccaactgatcttcagcatcttttactttcaccagcgtttctgggtgagcaaaaacaggaaggcaaaat
gccgcaaaaaagggaataagggcgacacggaaatgttgaatactcatactcttcctttttcaatattattgaagcatttatcagggttattgt
ctcatgagcggatacatatttgaatgtatttagaaaaataaacaaataggggttccgcgcacatttccccgaaaagtgccacctgaaattg
taaacgttaatattttgttaaaattcgcgttaaatttttgttaaatcagctcattttttaaccaataggccgaaatcggcaaaatcccttata
aatcaaaagaatagaccgagatagggttgagtgttgttccagtttggaacaagagtccactattaaagaacgtggactccaacgtcaaagg
gcgaaaaaccgtctatcagggcgatggcccactacgtgaaccatcaccctaatcaagttttttggggtcgaggtgccgtaaagcactaa
atcggaaccctaaagggagcccccgatttagagcttgacggggaaagccggcgaacgtggcgagaaaggaagggaagaaagcg
aaaggagcgggcgctagggcgctggcaagtgtagcggtcacgctgcgcgtaaccaccacacccgccgcgcttaatgcgccgctac
agggcgcgtc
SEQ ID NO: 23-Artificial scqucncc (Vcctcr 3; 8305 nt)
cattcgccattcaggctgcaaataagcgttgatattcagtcaattacaaacattaataacgaagagatgacagaaaaattttcattctgtga
cagagaaaaagtagccgaagatgacggtttgtcacatggagttggcaggatgtttgattaaaaacataacaggaagaaaaatgccccg
ctgtgggcggacaaaatagttgggaactgggaggggtggaaatggagtttttaaggattatttagggaagagtgacaaaatagatggg
aactgggtgtagcgtcgtaagctaatacgaaaattaaaaatgacaaaatagtttggaactagatttcacttatctggttcggatctcctaga
gcttacagcttcctgcaggcagctgcgcgctcgctcgctcactgaggccgcccgggcaaagcccgggcgtcgggcgacctttggtc
gcccggcctcagtgagcgagcgagcgcgcagagagggagtggccaactccatcactaggggttcctgcggccgcacgcgtgttac
taggttctagaaccggtgacgtctcccatggtgaagcttggatctgagatatcaaactgcctgtttgggcttctcatttcttacctccccttc
cctctcccacctgctactgggtgcatctctgctccccccttccccagcagatggttacctttgggctgttgctttcttgtcaccatctgagttc
tcagacgctggaaagccatgttctcggctctgtgaatgacaatgctgactggagtgctgcccctctgtaaagggctgggtgtggatggt
cacaagcccctcacatgcctcagccaagaggaagtagtacaggggtcagcccagaggtccaggggaaaggagtggaaaccgattt
ccccaccaagggaggggcctgtacctcagctgttcccatagcttacttgccacaactgccaagcaagtttcgctgagtttgacacatgg
atccctgtggatcaactgccctaggactccgtttgcacccatgtgacactgttgactttgccctgacgaagcagggccaacagtccccta
acttaattacaaaaactaatgactaagagagaggtggctagagctgaggcccctgagtcaggctgtggggggatcatctccagtaca
ggaagtgagactttcatttcctcctttccaagagagggctgagggagcagggttgagcaactggtgcagacagcctagctggactttg
ggtgaggcggttcagccatatcgaattctgctggggctactggcaggtaaggaggaaggaggctgaggggagggggcccctggga
gggagcctgccctgggttgctaaccatctcctctctgccaaaagtccggaaagccaccatggagcccctgaggctgctgatcctgctg
tttgtgacagaactgtctggagcccacaacaccacagtgttccagggagttgctggccagtctctgcaagtgtcttgcccctatgacagc
atgaagcactggggaaggaggaaggcttggtgtaggcagctgggagagaaaggaccttgccagagggtggtgagcacacacaac
ctgtggctgctgagcttcctcagaaggtggaatggctctacagccatcacagatgacaccctgggtggcaccctcaccatcaccctga
ggaacctgcagcctcatgatgctggcctgtaccaatgccagagcctgcatggctctgaggctgataccctcaggaaggtgttggtgga
ggtgctggctgatcctctggatcacagggatgctggagacctgtggttcccaggagagtctgagagctttgaggatgcccatgtggag
cacagcatcagcaggtctcttctggagggagagatccccttccctcccacaagcatcctgttgctgcttgcctgcatcttcctcatcaaga
tccttgctgcttctgctctttgggctgctgcctggcatggccagaaacctggaacacatcctccctctgaactggactgtggccatgacc
ctggctaccagttgcaaaccttgcctggcttgagggacacctgacaattgttaattaagtttaaaccctcgaggccgcaagcttatcgata
atcaacctctggattacaaaatttgtgaaagattgactggtattcttaactatgttgctccttttacgctatgtggatacgctgctttaatgc
ctttgtatcatgctattgcttcccgtatggctttcattttctcctccttgtataaatcctggttgctgtctctttatgaggagttgtggccc
gttgtcaggcaacgtggcgtggtgtgcactgtgtttgctgacgcaacccccactggttggggcattgccaccacctgtcagctcctttccggg
actttcgctttccccctccctattgccacggcggaactcatcgccgcctgccttgcccgctgctggacaggggctcggctgttgggcactgac
aattccgtggtgttgtcggggaaatcatcgtcctttccttggctgctcgcctgtgttgccacctggattctgcgcgggacgtccttctgcta
cgtcccttcggccctcaatccagcggaccttccttcccgcggcctgctgccggctctgcggcctcttccgcgtcttcgccttcgccctca
gacgagtcggatctccctttgggccgcctccccgcatcgataccgtcgactagagctcgctgatcagcctcgactgtgccttctagttgc
cagccatctgttgtttgcccctcccccgtgccttccttgaccctggaaggtgccactcccactgtcctttcctaataaaatgaggaaattgc
atcgcattgtctgagtaggtgtcattctattctggggggtggggggggcaggacagcaagggggaggattgggaagacaatagcag
gcatgctggggagagatccacgataacaaacagcttttttggggtgaacatattgactgaattcccgtgcggaccggcagtcctcactt
acctgagtgccctgttctgcactcctctcctccccaccctgcccatattcttcacaccctccttccctccccagtatcaaaacctaccctca
gactctttctgctgtgagactgataaggggctggtggactctggttgcattggaagggaaggagtgcttacagtggcatcccaggcact
agctggcagttgggggaagctttggggggcactggcactggtaatagcctctgaaattataagcctctcatttgaagcccctacagttat
aaaggaggaaagaacctgaggtgattcatctgcatctttggggcactctctcccctgctctgaaggtccccttgtcctcagctcttgtgag
gaggtcaggagcatagagatttccattgctgagttgaggggaacttacacaccaaatttttgtacacaattgcttcttcccactgaggaaa
gcacaccttttgctcccccagagcactggacaaacctgggcaagaggagagacagtgcctgaaagagtcttgttcctgcagcttctta
gtgagtggtcagggggaaggcagggttctgaggctgagtgaggaggtggtcagtgaggagagggccacagcccatctggtctctct
agttccaaaaggcaagccaccaacttcccaatcctatctttcaagcctctgtcacagttgaagttgaagtgagcaggcaagggaggac
caagagagaagtcaaagcagggggctctgggggctgccccagcaacaggctggtgctctaagcccatctcccccaccctaaggag
ggttcccaaaatagcagcctctcatctcccccaaaatatctccaagacaggtccttcctgaaaggggaacaaagccacagaaataggg
aagctggaagttaaaggtcaggaaagtcagtacctgtgaaggcagctgcagagcaagcaagagtggcagggcagggagagccag
ccccaggccaagaggagaggtcccagtccctgtgaaaagcaaagagtgtgacagaacagaactggagggggttttaagaccaagt
gcctccagaatagacccagagaaggtcagttacttctccaaggaggcccagcaagagcaacagtgagaaaaccaaacccaggtcc
caggtttcctggttcccaatttcccacaaacacttactgtgccatccactcccaacttgtataagaacctaaggcggaccgagcggccgc
aggaacccctagtgatggagttggccactccctctctgcgcgctcgctcgctcactgaggccgggcgaccaaaggtcgcccgacgc
ccgggctttgcccgggcggcctcagtgagcgagcgagcgcgcagctgcctgcaggcatgcaagctgtagccaaccactagaactat
agctagagtcctgggcgaacaaacgatgctcgccttccagaaaaccgaggatgcgaaccacttcatccggggtcagcaccaccggc
aagcgccgcgacggccgaggtcttccgatctcctgaagccagggcagatccgtgcacagcaccttgccgtagaagaacagcaagg
ccgccaatgcctgacgatgcgtggagaccgaaaccttgcgctcgttcgccagccaggacagaaatgcctcgacttcgctgctgccca
aggttgccgggtgacgcacaccgtggaaacggatgaaggcacgaacccagttgacataagcctgttcggttcgtaaactgtaatgca
agtagcgtatgcgctcacgcaactggtccagaaccttgaccgaacgcagcggtggtaacggcgcagtggcggttttcatggcttgttat
gactgtttttttgtacagtctatgcctcgggcatccaagcagcaagcgcgttacgccgtgggtcgatgtttgatgttatggagcagcaacg
atgttacgcagcagcaacgatgttacgcagcagggcagtcgccctaaaacaaagttaggtggctcaagtatgggcatcattcgcacat
gtaggctcggccctgaccaagtcaaatccatgcgggctgctcttgatcttttcggtcgtgagttcggagacgtagccacctactcccaac
atcagccggactccgattacctcgggaacttgctccgtagtaagacattcatcgcgcttgctgccttcgaccaagaagcggttgttggc
gctctcgcggcttacgttctgcccaggtttgagcagccgcgtagtgagatctatatctatgatctcgcagtctccggcgagcaccggag
gcagggcattgccaccgcgctcatcaatctcctcaagcatgaggccaacgcgcttggtgcttatgtgatctacgtgcaagcagattacg
gtgacgatcccgcagtggctctctatacaaagttgggcatacgggaagaagtgatgcactttgatatcgacccaagtaccgccacctaa
caattcgttcaagccgagatcggcttcccggccgcggagttgttcggtaaattgtcacaacgccgcgaatatagtctttaccatgccctt
ggccacgcccctctttaatacgacgggcaatttgcacttcagaaaatgaagagtttgctttagccataacaaaagtccagtatgctttttca
cagcataactggactgatttcagtttacaactattctgtctagtttaagactttattgtcatagtttagatctattttgttcagtttaagact
ttattgtccgcccacacccgcttacgcagggcatccatttattactcaaccgtaaccgattttgccaggttacgcggctggtctgcggtgtga
aataccgcacagatgcgtaaggagaaaataccgcatcaggcgctcttccgcttcctcgctcactgactcgctgcgctcggtcgttcggctg
cggcgagcggtatcagctcactcaaaggcggtaatacggttatccacagaatcaggggataacgcaggaaagaacatgtgagcaaa
aggccagcaaaaggccaggaaccgtaaaaaggccgcgttgctggcgtttttccataggctccgcccccctgacgagcatcacaaaa
atcgacgctcaagtcagaggtggcgaaacccgacaggactataaagataccaggcgtttccccctggaagctccctcgtgcgctctc
ctgttccgaccctgccgcttaccggatacctgtccgcctttctcccttcgggaagcgtggcgctttctcaatgctcacgctgtaggtatctc
agttcggtgtaggtcgttcgctccaagctgggctgtgtgcacgaaccccccgttcagcccgaccgctgcgccttatccggtaactatcg
tcttgagtccaacccggtaagacacgacttatcgccactggcagcagccactggtaacaggattagcagagcgaggtatgtaggcgg
tgctacagagttcttgaagtggtggcctaactacggctacactagaaggacagtatttggtatctgcgctctgctgaagccagttaccttc
ggaaaaagagttggtagctcttgatccggcaaacaaaccaccgctggtagcggtggtttttttgtttgcaagcagcagattacgcgcag
aaaaaaaggatctcaagaagatcctttgatcttttctacggggtctgacgctcagtggaacgaaaactcacgttaagggattttggtcatg
agattatcaaaaaggatcttcacctagatccttttaaattaaaaatgaagttttaaatcaatctaaagtatatatgagtaaacttggtctgaca
gttaccaatgcttaatcagtgaggcacctatctcagcgatctgtctatttcgttcatccatagttgcctgactccccgtcgtgtagataacta
cgatacgggagggcttaccatctggccccagtgctgcaatgataccgcgagacccacgctcaccggctccagatttatcagcaataaa
ccagccagccggaagggccgagcgcagaagtggtcctgcaactttatccgcctccatccagtctattaattgttgccgggaagctaga
gtaagtagttcgccagttaatagtttgcgcaacgttgttgccattgctacaggcatcgtggtgtcacgctcgtcgtttggtatggcttcattc
agctccggttcccaacgatcaaggcgagttacatgatcccccatgttgtgcaaaaaagcggttagctccttcggtcctccgatcgttgtc
agaagtaagttggccgcagtgttatcactcatggttatggcagcactgcataattctcttactgtcatgccatccgtaagatgcttttctgtg
actggtgagtactcaaccaagtcattctgagaatagtgtatgcggcgaccgagttgctcttgcccggcgtcaatacgggataataccgc
gccacatagcagaactttaaaagtgctcatcattggaaaacgttcttcggggcgaaaactctcaaggatcttaccgctgttgagatccag
ttcgatgtaacccactcgtgcacccaactgatcttcagcatcttttactttcaccagcgtttctgggtgagcaaaaacaggaaggcaaaat
gccgcaaaaaagggaataagggcgacacggaaatgttgaatactcatactcttcctttttcaatattattgaagcatttatcagggttattgt
ctcatgagcggatacatatttgaatgtatttagaaaaataaacaaataggggttccgcgcacatttccccgaaaagtgccacctgaaattg
taaacgttaatattttgttaaaattcgcgttaaatttttgttaaatcagctcattttttaaccaataggccgaaatcggcaaaatcccttata
aatcaaaagaatagaccgagatagggttgagtgttgttccagtttggaacaagagtccactattaaagaacgtggactccaacgtcaaagg
gcgaaaaaccgtctatcagggcgatggcccactacgtgaaccatcaccctaatcaagttttttggggtcgaggtgccgtaaagcactaa
atcggaaccctaaagggagcccccgatttagagcttgacggggaaagccggcgaacgtggcgagaaaggaagggaagaaagcg
aaaggagcgggcgctagggcgctggcaagtgtagcggtcacgctgcgcgtaaccaccacacccgccgcgcttaatgcgccgctac
agggcgcgtc
SEQ ID NO: 24-Artificial scqucncc (Vcctcr 4; 8305 nt)
cctgcaggcagctgcgcgctcgctcgctcactgaggccgcccgggcaaagcccgggcgtcgggcgacctttggtcgcccggcctc
agtgagcgagcgagcgcgcagagagggagtggccaactccatcactaggggttcctgcggccgcacgcgtgttactaggttctaga
accggtgacgtctcccatggtgaagcttggatctgagatatcaaactgcctgtttgggcttctcatttcttacctccccttccctctcccacc
tgctactgggtgcatctctgctccccccttccccagcagatggttacctttgggctgttgctttcttgtcaccatctgagttctcagacgctg
gaaagccatgttctcggctctgtgaatgacaatgctgactggagtgctgcccctctgtaaagggctgggtgtggatggtcacaagcccc
tcacatgcctcagccaagaggaagtagtacaggggtcagcccagaggtccaggggaaaggagtggaaaccgatttccccaccaag
ggaggggcctgtacctcagctgttcccatagcttacttgccacaactgccaagcaagtttcgctgagtttgacacatggatccctgtgga
tcaactgccctaggactccgtttgcacccatgtgacactgttgactttgccctgacgaagcagggccaacagtcccctaacttaattaca
aaaactaatgactaagagagaggtggctagagctgaggcccctgagtcaggctgtgggtgggatcatctccagtacaggaagtgag
actttcatttcctcctttccaagagagggctgagggagcagggttgagcaactggtgcagacagcctagctggactttgggtgaggcg
gttcagccatatcgaattctgctggggctactggcaggtaaggaggaaggaggctgaggggagggggcccctgggagggagcctg
ccctgggttgctaaccatctcctctctgccaaaagtccggaaagccaccatggagcccctgagactgctcatcctgctgtttgtgacaga
actgtctggggcccacaacaccacagtgttccagggggtggctggccagtccctccaggtgtcctgcccctatgactccatgaagcac
tgggggagaagaaaggcttggtgtagacagctgggggagaaagggccttgccagagagtggtgtccacacacaacctgtggctgct
gtccttcctgagaagatggaatggctccacagccatcacagatgacaccctggggggcaccctcaccatcaccctgagaaacctgca
gcctcatgatgctggcctgtaccagtgccagtccctgcatggctctgaggctgataccctgagaaaggtgctggtggaggtgctggct
gatcctctggatcacagagatgctggggacctgtggttcccaggggagtctgagtcctttgaggatgcccatgtggagcactccatctc
cagatccctgctggagggggagatccccttccctcccacatccatcctgctgctgctggcctgcatcttcctcatcaagatcctggctgc
ttctgctctgtgggctgctgcctggcatggccagaaacctgggacacatcctccctctgaactggactgtggccatgaccctggctacc
agctgcagaccctgcctggcctgagagacacctgacaattgttaattaagtttaaaccctcgaggccgcaagcttatcgataatcaacct
ctggattacaaaatttgtgaaagattgactggtattcttaactatgttgctccttttacgctatgtggatacgctgctttaatgcctttgtat
catgctattgcttcccgtatggctttcattttctcctccttgtataaatcctggttgctgtctctttatgaggagttgtggcccgttgtcagg
caacgtggcgtggtgtgcactgtgtttgctgacgcaacccccactggttggggcattgccaccacctgtcagctcctttccgggactttcgct
ttccccctccctattgccacggcggaactcatcgccgcctgccttgcccgctgctggacaggggctcggctgttgggcactgacaattccgt
ggtgttgtcggggaaatcatcgtcctttccttggctgctcgcctgtgttgccacctggattctgcgcgggacgtccttctgctacgtccctt
cggccctcaatccagcggaccttccttcccgcggcctgctgccggctctgcggcctcttccgcgtcttcgccttcgccctcagacgagt
cggatctccctttgggccgcctccccgcatcgataccgtcgactagagctcgctgatcagcctcgactgtgccttctagttgccagccat
ctgttgtttgcccctcccccgtgccttccttgaccctggaaggtgccactcccactgtcctttcctaataaaatgaggaaattgcatcgcatt
gtctgagtaggtgtcattctattctggggggtggggggggcaggacagcaagggggaggattgggaagacaatagcaggcatgct
ggggagagatccacgataacaaacagcttttttggggtgaacatattgactgaattcccgtgcggaccggcagtcctcacttacctgag
tgccctgttctgcactcctctcctccccaccctgcccatattcttcacaccctccttccctccccagtatcaaaacctaccctcagactctttc
tgctgtgagactgataaggggctggtggactctggttgcattggaagggaaggagtgcttacagtggcatcccaggcactagctggca
gttgggggaagctttggggggcactggcactggtaatagcctctgaaattataagcctctcatttgaagcccctacagttataaaggag
gaaagaacctgaggtgattcatctgcatctttggggcactctctcccctgctctgaaggtccccttgtcctcagctcttgtgaggaggtca
ggagcatagagatttccattgctgagttgaggggaacttacacaccaaatttttgtacacaattgcttcttcccactgaggaaagcacacc
ttttgctcccccagagcactggacaaacctgggcaagaggagagacagtgcctgaaagagtcttgttcctgcagcttcttagtgagtgg
tcagggggaaggcagggttctgaggctgagtgaggaggtggtcagtgaggagagggccacagcccatctggtctctctagttccaa
aaggcaagccaccaacttcccaatcctatctttcaagcctctgtcacagttgaagttgaagtgagcaggcaagggaggaccaagaga
gaagtcaaagcagggggctctgggggctgccccagcaacaggctggtgctctaagcccatctcccccaccctaaggagggttccca
aaatagcagcctctcatctcccccaaaatatctccaagacaggtccttcctgaaaggggaacaaagccacagaaatagggaagctgg
aagttaaaggtcaggaaagtcagtacctgtgaaggcagctgcagagcaagcaagagtggcagggcagggagagccagccccagg
ccaagaggagaggtcccagtccctgtgaaaagcaaagagtgtgacagaacagaactggagggggttttaagaccaagtgcctccag
aatagacccagagaaggtcagttacttctccaaggaggcccagcaagagcaacagtgagaaaaccaaacccaggtcccaggtttcct
ggttcccaatttcccacaaacacttactgtgccatccactcccaacttgtataagaacctaaggggaccgagcggccgcaggaaccc
ctagtgatggagttggccactccctctctgcgcgctcgctcgctcactgaggccgggcgaccaaaggtcgcccgacgcccgggcttt
gcccgggcggcctcagtgagcgagcgagcgcgcagctgcctgcaggcatgcaagctgtagccaaccactagaactatagctagag
tcctgggcgaacaaacgatgctcgccttccagaaaaccgaggatgcgaaccacttcatccggggtcagcaccaccggcaagcgcc
gcgacggccgaggtcttccgatctcctgaagccagggcagatccgtgcacagcaccttgccgtagaagaacagcaaggccgccaat
gcctgacgatgcgtggagaccgaaaccttgcgctcgttcgccagccaggacagaaatgcctcgacttcgctgctgcccaaggttgcc
gggtgacgcacaccgtggaaacggatgaaggcacgaacccagttgacataagcctgttcggttcgtaaactgtaatgcaagtagcgt
atgcgctcacgcaactggtccagaaccttgaccgaacgcagcggtggtaacggcgcagtggcggttttcatggcttgttatgactgtttt
tttgtacagtctatgcctcgggcatccaagcagcaagcgcgttacgccgtgggtcgatgtttgatgttatggagcagcaacgatgttacg
cagcagcaacgatgttacgcagcagggcagtcgccctaaaacaaagttaggtggctcaagtatgggcatcattcgcacatgtaggctc
ggacctgaccaagtcaaatccatgcgggctgctcttgatcttttcggtcgtgagttcggagacgtagccacctactcccaacatcagcc
ggactccgattacctcgggaacttgctccgtagtaagacattcatcgcgcttgctgccttcgaccaagaagcggttgttggcgctctcgc
ggcttacgttctgcccaggtttgagcagccgcgtagtgagatctatatctatgatctcgcagtctccggcgagcaccggaggcagggc
attgccaccgcgctcatcaatctcctcaagcatgaggccaacgcgcttggtgcttatgtgatctacgtgcaagcagattacggtgacgat
cccgcagtggctctctatacaaagttgggcatacgggaagaagtgatgcactttgatatcgacccaagtaccgccacctaacaattcgtt
caagccgagatcggcttcccggccgcggagttgttcggtaaattgtcacaacgccgcgaatatagtctttaccatgcccttggccacgc
ccctctttaatacgacgggcaatttgcacttcagaaaatgaagagtttgctttagccataacaaaagtccagtatgctttttcacagcataa
ctggactgatttcagtttacaactattctgtctagtttaagactttattgtcatagtttagatctattttgttcagtttaagactttattgt
ccgcccacacccgcttacgcagggcatccatttattactcaaccgtaaccgattttgccaggttacgcggctggtctgcggtgtgaaatacc
gcacagatgcgtaaggagaaaataccgcatcaggcgctcttccgcttcctcgctcactgactcgctgcgctcggtcgttcggctgcggcgagc
ggtatcagctcactcaaaggcggtaatacggttatccacagaatcaggggataacgcaggaaagaacatgtgagcaaaaggccagc
aaaaggccaggaaccgtaaaaaggccgcgttgctggcgtttttccataggctccgcccccctgacgagcatcacaaaaatcgacgct
caagtcagaggtggcgaaacccgacaggactataaagataccaggcgtttccccctggaagctccctcgtgcgctctcctgttccgac
cctgccgcttaccggatacctgtccgcctttctcccttcgggaagcgtggcgctttctcaatgctcacgctgtaggtatctcagttcggtgt
aggtcgttcgctccaagctgggctgtgtgcacgaaccccccgttcagcccgaccgctgcgccttatccggtaactatcgtcttgagtcc
aacccggtaagacacgacttatcgccactggcagcagccactggtaacaggattagcagagcgaggtatgtaggcggtgctacaga
gttcttgaagtggtggcctaactacggctacactagaaggacagtatttggtatctgcgctctgctgaagccagttaccttcggaaaaag
agttggtagctcttgatccggcaaacaaaccaccgctggtagcggtggtttttttgtttgcaagcagcagattacgcgcagaaaaaaag
gatctcaagaagatcctttgatcttttctacggggtctgacgctcagtggaacgaaaactcacgttaagggattttggtcatgagattatca
aaaaggatcttcacctagatccttttaaattaaaaatgaagttttaaatcaatctaaagtatatatgagtaaacttggtctgacagttaccaat
gcttaatcagtgaggcacctatctcagcgatctgtctatttcgttcatccatagttgcctgactccccgtcgtgtagataactacgatacgg
gagggcttaccatctggccccagtgctgcaatgataccgcgagacccacgctcaccggctccagatttatcagcaataaaccagcca
gccggaagggccgagcgcagaagtggtcctgcaactttatccgcctccatccagtctattaattgttgccgggaagctagagtaagta
gttcgccagttaatagtttgcgcaacgttgttgccattgctacaggcatcgtggtgtcacgctcgtcgtttggtatggcttcattcagctccg
gttcccaacgatcaaggcgagttacatgatcccccatgttgtgcaaaaaagcggttagctccttcggtcctccgatcgttgtcagaagta
agttggccgcagtgttatcactcatggttatggcagcactgcataattctcttactgtcatgccatccgtaagatgcttttctgtgactggtg
agtactcaaccaagtcattctgagaatagtgtatgcggcgaccgagttgctcttgcccggcgtcaatacgggataataccgcgccacat
agcagaactttaaaagtgctcatcattggaaaacgttcttcggggcgaaaactctcaaggatcttaccgctgttgagatccagttcgatgt
aacccactcgtgcacccaactgatcttcagcatcttttactttcaccagcgtttctgggtgagcaaaaacaggaaggcaaaatgccgcaa
aaaagggaataagggcgacacggaaatgttgaatactcatactcttcctttttcaatattattgaagcatttatcagggttattgtctcatga
gcggatacatatttgaatgtatttagaaaaataaacaaataggggttccgcgcacatttccccgaaaagtgccacctgaaattgtaaacgt
taatattttgttaaaattcgcgttaaatttttgttaaatcagctcattttttaaccaataggccgaaatcggcaaaatcccttataaatcaaa
agaatagaccgagatagggttgagtgttgttccagtttggaacaagagtccactattaaagaacgtggactccaacgtcaaagggcgaaaa
accgtctatcagggcgatggcccactacgtgaaccatcaccctaatcaagttttttggggtcgaggtgccgtaaagcactaaatcggaa
ccctaaagggagcccccgatttagagcttgacggggaaagccggcgaacgtggcgagaaaggaagggaagaaagcgaaaggag
cgggcgctagggcgctggcaagtgtagcggtcacgctgcgcgtaaccaccacacccgccgcgcttaatgcgccgctacagggcgc
gtccattcgccattcaggctgcaaataagcgttgatattcagtcaattacaaacattaataacgaagagatgacagaaaaattttcattctg
tgacagagaaaaagtagccgaagatgacggtttgtcacatggagttggcaggatgtttgattaaaaacataacaggaagaaaaatgcc
ccgctgtgggcggacaaaatagttgggaactgggaggggtggaaatggagtttttaaggattatttagggaagagtgacaaaatagat
gggaactgggtgtagcgtcgtaagctaatacgaaaattaaaaatgacaaaatagtttggaactagatttcacttatctggttcggatctcct
agagcttacagctt
SEQ ID NO: 25-Artificial scqucncc (Vcctcr 5; 2804 nt)
cctgcaggcagctgcgcgctcgctcgctcactgaggccgcccgggcaaagcccgggcgtcgggcgacctttggtcgcccggcctc
agtgagcgagcgagcgcgcagagagggagtggccaactccatcactaggggttcctgcggccgcacgcgtactaggttctagaac
cggtgacgtctcccatggtgaagcttggatctgagatatcaaactgcctgtttgggcttctcatttcttacctccccttccctctcccacctg
ctactgggtgcatctctgctccccccttccccagcagatggttacctttgggctgttgctttcttgtcaccatctgagttctcagacgctgga
aagccatgttctcggctctgtgaatgacaatgctgactggagtgctgcccctctgtaaagggctgggtgtggatggtcacaagcccctc
acatgcctcagccaagaggaagtagtacaggggtcagcccagaggtccaggggaaaggagtggaaaccgatttccccaccaagg
gaggggcctgtacctcagctgttcccatagcttacttgccacaactgccaagcaagtttcgctgagtttgacacatggatccctgtggat
caactgccctaggactccgtttgcacccatgtgacactgttgactttgccctgacgaagcagggccaacagtcccctaacttaattacaa
aaactaatgactaagagagaggtggctagagctgaggcccctgagtcaggctgtggggggatcatctccagtacaggaagtgaga
ctttcatttcctcctttccaagagagggctgagggagcagggttgagcaactggtgcagacagcctagctggactttgggtgaggcggt
tcagccatatcgaattctgctggggctactggcaggtaaggaggaaggaggctgaggggagggggcccctgggagggagcctgc
cctgggttgctaaccatctcctctctgccaaaagtccggaaagccaccatggagcccctgcgcctgctgatcctgctgttcgtgaccga
gctgagcggcgcccacaacaccaccgtgttccagggcgtggccggccagagcctgcaggtgagctgcccctacgacagcatgaa
gcactggggccgccgcaaggcctggtgccgccagctgggcgagaagggcccctgccagcgcgtggtgagcacccacaacctgtg
gctgctgagcttcctgcgccgctggaacggcagcaccgccatcaccgacgacaccctgggcggcaccctgaccatcaccctgcgc
aacctgcagccccacgacgccggcctgtaccagtgccagagcctgcacggcagcgaggccgacaccctgcgcaaggtgctggtg
gaggtgctggccgaccccctggaccaccgcgacgccggcgacctgtggttccccggcgagagcgagagcttcgaggacgcccac
gtggagcacagcatcagccgcagcctgctggagggcgagatccccttcccccccaccagcatcctgctgctgctggcctgcatcttc
ctgatcaagatcctggccgccagcgccctgtgggccgccgcctggcacggccagaagcccggcacccacccccccagcgagctg
gactgcggccacgaccccggctaccagctgcagaccctgcccggcctgcgcgacacctgacaattgttaattaagtttaaaccctcga
ggccgcaagcttatcgataatcaacctctggattacaaaatttgtgaaagattgactggtattcttaactatgttgctccttttacgctatgtg
gatacgctgctttaatgcctttgtatcatgctattgcttcccgtatggctttcattttctcctccttgtataaatcctggttgctgtctcttt
atgaggagttgtggcccgttgtcaggcaacgtggcgtggtgtgcactgtgtttgctgacgcaacccccactggttggggcattgccaccacctg
tcagctcctttccgggactttcgctttccccctccctattgccacggcggaactcatcgccgcctgccttgcccgctgctggacaggggc
tcggctgttgggcactgacaattccgtggtgttgtcggggaaatcatcgtcctttccttggctgctcgcctgtgttgccacctggattctgc
gcgggacgtccttctgctacgtcccttcggccctcaatccagcggaccttccttcccgcggcctgctgccggctctgcggcctcttccg
cgtcttcgccttcgccctcagacgagtcggatctccctttgggccgcctccccgcatcgataccgtcgactagagctcgctgatcagcc
tcgactgtgccttctagttgccagccatctgttgtttgcccctcccccgtgccttccttgaccctggaaggtgccactcccactgtcctttcc
taataaaatgaggaaattgcatcgcattgtctgagtaggtgtcattctattctggggggtggggggggcaggacagcaagggggagg
attgggaagacaatagcaggcatgctggggagagatccacgataacaaacagcttttttggggtgaacatattgactgaattcccgtgc
ggaccgagcggccgcaggaacccctagtgatggagttggccactccctctctgcgcgctcgctcgctcactgaggccgggcgacca
aaggtcgcccgacgcccgggctttgcccgggcggcctcagtgagcgagcgagcgcgcagctgcctgcagg
SEQ ID NO: 26-Artificial scqucncc (Vcctcr 6; 4048 nt)
ttggccactccctctctgcgcgctcgctcgctcactgaggccgcccgggcaaagcccgggcgtcgggcgacctttggtcgcccggc
ctcagtgagcgagcgagcgcgcagagagggagtggccaactccatcactaggggttcctgcggccgcacgcgtactaggttctaga
accggtgacgtctcccatggtgaagcttggatctgagatatcaaactgcctgtttgggcttctcatttcttacctccccttccctctcccacc
tgctactgggtgcatctctgctccccccttccccagcagatggttacctttgggctgttgctttcttgtcaccatctgagttctcagacgctg
gaaagccatgttctcggctctgtgaatgacaatgctgactggagtgctgcccctctgtaaagggctgggtgtggatggtcacaagcccc
tcacatgcctcagccaagaggaagtagtacaggggtcagcccagaggtccaggggaaaggagtggaaaccgatttccccaccaag
ggaggggcctgtacctcagctgttcccatagcttacttgccacaactgccaagcaagtttcgctgagtttgacacatggatccctgtgga
tcaactgccctaggactccgtttgcacccatgtgacactgttgactttgccctgacgaagcagggccaacagtcccctaacttaattaca
aaaactaatgactaagagagaggtggctagagctgaggcccctgagtcaggctgtgggtgggatcatctccagtacaggaagtgag
actttcatttcctcctttccaagagagggctgagggagcagggttgagcaactggtgcagacagcctagctggactttgggtgaggcg
gttcagccatatcgaattctgctggggctactggcaggtaaggaggaaggaggctgaggggagggggcccctgggagggagcctg
ccctgggttgctaaccatctcctctctgccaaaagtccggaaagccaccatggagcccctgaggctgctcatcctgctgtttgtgacag
aactgtctggagcccacaacaccacagtgttccagggagttgctggccagtctctgcaagtgtcttgcccctatgacagcatgaagcac
tggggaaggaggaaggcttggtgtaggcagctgggagagaaaggaccttgccagagggtggtgagcacacacaacctgtggctgc
tgagcttcctcagaaggtggaatggctctacagccatcacagatgacaccctgggtggcaccctcaccatcaccttaaggaacctgca
gcctcatgatgctggcctgtaccaatgccagagcctgcatggctctgaggctgataccctcaggaaggtgttggtggaggtgctggct
gatcctctggatcacagggatgctggagacctgtggttcccaggagagtctgagagctttgaggatgcccatgtggagcacagcatca
gcaggtctcttctggagggagagatccccttccctcccacaagcatcctgttgctgcttgcctgcatcttcctgatcaagatccttgctgct
tctgctctttgggctgctgcctggcatggccagaaacctggaacacatcctccctctgaactggactgtggccatgaccctggctacca
gttgcaaaccttgcctggcttgagggacacctgacaattgttaattaagtttaaaccctcgaggccgcaagcttatcgataatcaacctct
ggattacaaaatttgtgaaagattgactggtattcttaactatgttgctccttttacgctatgtggatacgctgctttaatgcctttgtatca
tgctattgcttcccgtatggctttcattttctcctccttgtataaatcctggttgctgtctctttatgaggagttgtggcccgttgtcaggca
acgtggcgtggtgtgcactgtgtttgctgacgcaacccccactggttggggcattgccaccacctgtcagctcctttccgggactttcgcttt
ccccctccctattgccacggcggaactcatcgccgcctgccttgcccgctgctggacaggggctcggctgttgggcactgacaattccgtgg
tgttgtcggggaaatcatcgtcctttccttggctgctcgcctgtgttgccacctggattctgcgcgggacgtccttctgctacgtcccttcg
gccctcaatccagcggaccttccttcccgcggcctgctgccggctctgcggcctcttccgcgtcttcgccttcgccctcagacgagtcg
gatctccctttgggccgcctccccgcatcgataccgtcgactagagctcgctgatcagcctcgactgtgccttctagttgccagccatct
gttgtttgcccctcccccgtgccttccttgaccctggaaggtgccactcccactgtcctttcctaataaaatgaggaaattgcatcgcattg
tctgagtaggtgtcattctattctggggggtggggggggcaggacagcaagggggaggattgggaagacaatagcaggcatgctg
gggagagatccacgataacaaacagcttttttggggtgaacatattgactgaattcccgtgcggaccggcagtcctcacttacctgagt
gccctgttctgcactcctctcctccccaccctgcccatattcttcacaccctccttccctccccagtatcaaaacctaccctcagactctttct
gctgtgagactgataaggggctggtggactctggttgcattggaagggaaggagtgcttacagtggcatcccaggcactagctggca
gttgggggaagctttggggggcactggcactggtaatagcctctgaaattataagcctctcatttgaagcccctacagttataaaggag
gaaagaacctgaggtgattcatctgcatctttggggcactctctcccctgctctgaaggtccccttgtcctcagctcttgtgaggaggtca
ggagcatagagatttccattgctgagttgaggggaacttacacaccaaatttttgtacacaattgcttcttcccactgaggaaagcacacc
ttttgctcccccagagcactggacaaacctgggcaagaggagagacagtgcctgaaagagtcttgttcctgcagcttcttagtgagtgg
tcagggggaaggcagggttctgaggctgagtgaggaggtggtcagtgaggagagggccacagcccatctggtctctctagttccaa
aaggcaagccaccaacttcccaatcctatctttcaagcctctgtcacagttgaagttgaagtgagcaggcaagggaggaccaagaga
gaagtcaaagcagggggctctgggggctgccccagcaacaggctggtgctctaagcccatctcccccaccctaaggagggttccca
aaatagcagcctctcatctcccccaaaatatctccaagacaggtccttcctgaaaggggaacaaagccacagaaatagggaagctgg
aagttaaaggtcaggaaagtcagtacctgtgaaggcagctgcagagcaagcaagagtggcagggcagggagagccagccccagg
ccaagaggagaggtcccagtccctgtgaaaagcaaagagtgtgacagaacagaactggagggggttttaagaccaagtgcctccag
aatagacccagagaaggtcagttacttctccaaggaggcccagcaagagcaacagtgagaaaaccaaacccaggtcccaggtttcct
ggttcccaatttcccacaaacacttactgtgccatccactcccaacttgtataagaacctaaggggaccgagcggccgcaggaaccc
ctagtgatggagttggccactccctctctgcgcgctcgctcgctcactgaggccgggcgaccaaaggtcgcccgacgcccgggcttt
gcccgggcggcctcagtgagcgagcgagcgcgcagagagggagtggccaa

The invention claimed is:

1. A nucleic acid comprising a nucleotide sequence for a microglial-specific promoter operably linked to a nucleotide sequence encoding human triggering receptor expressed on myeloid cells 2 (TREM2), wherein the nucleotide sequence encoding human TREM2 is selected from any one of SEQ ID NOS:3 to 6.

2. The nucleic acid of claim 1, wherein the nucleotide sequence for the microglial-specific promoter is SEQ ID NO:8.

3. A vector comprising the nucleic acid of claim 1.

4. The vector of claim 3, wherein the vector is a recombinant adeno-associated virus (rAAV) vector.

5. A recombinant adeno-associated virus (rAAV) comprising:
(a) a rAAV vector comprising, in 5′ to 3′ order:
(i) a nucleotide sequence for a first AAV inverted terminal repeat (ITR) or a reverse complementary sequence thereto;
(ii) a nucleotide sequence for a microglial-specific promoter;
(iii) a nucleotide sequence for a triggering receptor expressed on myeloid cells 2 (TREM2)-encoding transgene, wherein the nucleotide sequence for the TREM2-encoding transgene is selected from the group consisting of SEQ ID NOS:3 to 6;
(iv) a nucleotide sequence for a post-transcriptional regulatory element;
(v) a nucleotide sequence for a polyadenylation signal; and
(vii) a nucleotide sequence for a second AAV ITR or a reverse complementary sequence thereto; and
(b) encapsidated in a modified AAV6 capsid comprising an amino acid sequence comprising T492V, Y705F and Y731F, mutations as compared to wild-type AAV6 capsid.

6. The rAAV of claim 5, wherein the nucleotide sequence for the first ITR sequence and the second ITR sequence independently are selected from SEQ ID NO:13 or 14, or a reverse complementary sequence thereto.

7. The rAAV of claim 5, wherein the nucleotide sequence for the microglial-specific promoter is SEQ ID NO:8.

8. The rAAV of claim 5, wherein the nucleotide sequence for the post-transcriptional regulatory element is SEQ ID NO:11.

9. The rAAV of claim 5, wherein the nucleotide sequence for the polyadenylation signal is SEQ ID NO:12.

10. The rAAV of claim 5, wherein the rAAV vector further comprises a nucleotide sequence for a stuffer sequence, and wherein the nucleotide sequence for the stuffer sequence is selected from the group consisting of SEQ ID NOS:16 to 18.

11. The rAAV of claim 5, wherein the rAAV vector is selected from the group consisting of SEQ ID NOS:21 to 26.

12. The rAAV of claim 5, wherein the amino acid sequence for the modified AAV6 capsid is SEQ ID NO:19.

13. A pharmaceutical composition comprising:
(a) the rAAV of claim 5; and
(b) a pharmaceutically acceptable carrier.

14. The pharmaceutical composition of claim 13 further comprising:
(c) about 20 mM TRIS (pH 8.0);
(d) about 1 mM MgCl₂;
(e) about 200 mM NaCl; and
(f) about 0.005% (w/v) Poloxamer 188.

15. The pharmaceutical composition of claim 13, wherein the rAAV is present at a concentration from about 1×10¹³ vector genomes (vg) to about 7×10¹⁴ vg.

16. A method of treating a triggering receptor expressed on myeloid cells 2 (TREM2)-associated disease or disorder in an individual, the method comprising the step of:
administering to the individual an effective amount of the rAAV of claim 5.

17. The method of claim 16, wherein the TREM2-associated disease or disorder is selected from the group consisting of Alzheimer's Disease (AD), adult-onset leukoencephalopathy with axonal spheroids and pigmented glia (ALSP), Nasu-Hakola Disease (NHD), frontotemporal dementia, amyotrophic lateral sclerosis (ALS), cognitive deficit, memory loss, spinal cord injury, traumatic brain injury and multiple sclerosis.

18. The method of claim 16, wherein the administering is via intracisternal magna (ICM) injection.

19. The method of claim 16, wherein the administering is via intravenous (IV) injection.

20. The method of claim 16, wherein the effect amount is about 1×10¹³ vector genomes (vg) to about 7×10¹⁴ vg.
